(19) United States
(12) Patent Application Publication
(10) Pub. No.: US 2026/0294956 A1
(43) Pub. Date: Oct. 1, 2026

(54) TREATMENT OF HEMOPHILIA WITH FITUSIRAN

(71) Applicant: Genzyme Corporation, Cambridge, MA (US)

(72) Inventors: Malidi AHAMADI, Chalfont, PA (US); Timothy CHOW, Jamaica Plain, MA (US); Marek DEMISSIE, Boston, MA (US); Erica FERRARI, Frankfurt am Main (DE); Dirk HOERTER, Frankfurt am Main (DE); Laurel MENAPACE, Monrovia, MD (US); Marja PUURUNEN, Dover, MA (US)

(21) Appl. No.: 19/573,679

(22) Filed: Mar. 20, 2026

Related U.S. Application Data
(60) Provisional application No. 63/775,921, filed on Mar. 21, 2025.
(60) Provisional application No. 63/776,914, filed on Mar. 24, 2025.
(60) Provisional application No. 63/778,311, filed on Mar. 26, 2025.
(60) Provisional application No. 63/779,198, filed on Mar. 27, 2025.
(60) Provisional application No. 63/818,424, filed on Jun. 5, 2025.
(60) Provisional application No. 63/961,054, filed on Jan. 15, 2026.
(60) Provisional application No. 63/976,755, filed on Feb. 5, 2026.

(51) Int. Cl. A61K 31/713; A61P 7/04; C12N 15/113

(52) U.S. Cl. CPC A61K 31/713; A61P 7/04; C12N 15/113; C12N 2310/14

(57) ABSTRACT

The present disclosure provides methods for using fitusiran to treat patients with hemophilia A or hemophilia B.

### CROSS-REFERENCE TO RELATED APPLICATIONS

This application claims priority to and benefit of U.S. Provisional Application No. 63/775,921, filed Mar. 21, 2025, U.S. Provisional Application No. 63/776,914, filed Mar. 24, 2025, U.S. Provisional Application No. 63/778,311, filed Mar. 26, 2025, U.S. Provisional Application No. 63/779,198, filed Mar. 27, 2025, U.S. Provisional Application No. 63/818,424, filed Jun. 5, 2025, U.S. Provisional Application No. 63/961,054, filed Jan. 15, 2026, and U.S. Provisional Application No. 63/976,755, filed Feb. 5, 2026, the contents of each of which are incorporated herein by reference in their entireties.

### REFERENCE TO AN ELECTRONIC SEQUENCE LISTING

The contents of the electronic sequence listing (159792022300SEQLIST.xml; Size: 42,408 bytes; and Date of Creation: Feb. 25, 2026) are herein incorporated by reference in their entirety.

### BACKGROUND OF THE INVENTION

Hemophilia A and hemophilia B are X-linked recessive inherited bleeding disorders, characterized by deficiency of coagulation Factor VIII (FVIII) or Factor IX (FIX), leading to a profound defect of thrombin generation with impaired hemostasis and increased risk of bleeding. Hemophilia A is found in approximately 1 in 4,000 males whereas hemophilia B is five times less common and seen in approximately 1 in 20,000 males. The disease phenotype presents similarly in hemophilia A and B.

Hemophilia is classified as mild (factor levels 6% to 30%), moderate (factor levels 1% to 5%), or severe (factor levels <1%) based on clotting factor activity relative to normal (healthy, non-hemophiliac plasma levels of factor are 50% to 150%). Patients with mild hemophilia typically experience bleeding after a serious injury or surgery. Patients with moderate hemophilia experience bleeding episodes associated with injuries, and may have spontaneous bleeding episodes. Severe hemophilia patients experience substantial bleeding with injury and may have frequent spontaneous bleeding episodes resulting in debilitating musculoskeletal damage that can markedly impair a patient's mobility and quality of life (QoL).

Antithrombin (AT) is a liver-expressed natural anticoagulant that plays a key role in inhibiting thrombin. AT acts as an inhibitor of FVIIa and FXa, which are typically at normal levels in patients with hemophilia A or B. Extensive preclinical in vitro and in vivo studies have described reduction of AT as a potential safe and effective way to correct thrombin generation in both hemophilia A and B and control against microvascular and macrovascular traumatic bleeding episodes. There remains a need for alternative treatments for patients having hemophilia, to prevent or reduce the frequency of bleeding episodes while reducing treatment burden, improving clinical outcomes, and reducing the risk of adverse events.

### SUMMARY OF THE INVENTION

The present disclosure provides a method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising: (a) obtaining a measurement of an antithrombin (AT) level in the human patient, wherein the human patient has previously received fitusiran at a starting dose of 50 mg about once every 2 months; (b) performing one of the following steps: (i) if the AT level is 15-35%, subcutaneously administering to the human patient fitusiran at a dose of 50 mg about once every 2 months, (ii) if the AT level is >35% after 6 months, subcutaneously administering to the human patient fitusiran at a dose of 50 mg about once every month, or (iii) if the AT level is <15%, subcutaneously administering to the human patient fitusiran at a dose of 20 mg about once every 2 months; (c) after administering to the human patient fitusiran at a dose of 20 mg about once every 2 months, obtaining a measurement of an antithrombin (AT) level in the human patient; (d) performing one of the following steps: (i) if the AT level is 15-35%, subcutaneously administering to the human patient fitusiran at a dose of 20 mg about once every 2 months, (ii) if the AT level is >35% after 6 months, subcutaneously administering to the human patient fitusiran at a dose of 20 mg about once every month, or (iii) if the AT level is <15%, subcutaneously administering to the human patient fitusiran at a dose of 10 mg about once every 2 months; (e) after administering to the human patient fitusiran at a dose of 10 mg about once every 2 months, obtaining a measurement of an antithrombin (AT) level in the human patient; and (f) performing one of the following steps: (i) if the AT level is 15-35%, subcutaneously administering to the human patient fitusiran at a dose of 10 mg about once every 2 months, (ii) if the AT level is >35% after 6 months, subcutaneously administering to the human patient fitusiran at a dose of 10 mg about once every month, or (iii) if the AT level is <15%, discontinuing or pausing fitusiran treatment. The present disclosure also provides a method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 20 mg about once every 2 months; (b) obtaining a measurement of an antithrombin (AT) level in the human patient; and (c) performing one of the following steps: (i) if the AT level is 15-35%, repeating step (a), (ii) if the AT level is >35% after 6 months, subcutaneously administering to the human patient fitusiran at a dose of 20 mg about once every month, or (iii) if the AT level is <15%, subcutaneously administering to the human patient fitusiran at a lower dose amount. In some embodiments, step (c)(iii) comprises subcutaneously administering to the human patient fitusiran at a dose of 10 mg about once every 2 months. In some embodiments, the fitusiran is in a pharmaceutically acceptable salt form. In some embodiments, the fitusiran is in a sodium salt form.

The present disclosure also provides a method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 10 mg about once every 2 months; (b) obtaining a measurement of an antithrombin (AT) level in the human patient; and (c) performing one of the following steps: (i) if the AT level is 15-35%, repeating step (a), (ii) if the AT level is >35% after 6 months, subcutaneously administering to the human patient fitusiran at a dose of 10 mg about once every month, or (iii) if the AT level is <15%, discontinuing or pausing fitusiran treatment. The present disclosure also provides a method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising subcutaneously administering to the human patient in need thereof fitusiran at a dose of: about 50 mg about once a month or about once every four weeks, about 20 mg about once a month or about once every four weeks, about 10 mg about once a month or about once every four weeks, about 50 mg about once every other month or about once every eight weeks, about 20 mg about once every other month or about once every eight weeks, or about 10 mg about once every other month or about once every eight weeks. The present disclosure also provides a method of reducing a risk of hepatotoxicity in a human patient with hemophilia A or B with or without factor VIII or factor IX inhibitors who receives fitusiran at a current dose for routine prophylaxis to prevent or reduce the frequency of bleeding episodes, the method comprising administering fitusiran at the current dose or a modified dose based on an antithrombin (AT) activity level in the human patient, wherein: (a) if the current dose is 50 mg every 2 months and the AT level is <15%, fitusiran is administered at a modified dose of 20 mg every 2 months; (b) if the current dose is 50 mg every 2 months and the AT level is 15-35%, fitusiran is administered at the current dose; (c) if the current dose is 50 mg every 2 months and the AT level is >35% after 6 months, fitusiran is administered at a modified dose of 50 mg every month; (d) if the current dose is 20 mg every 2 months and the AT level is <15%, fitusiran is administered at a modified dose of 10 mg every 2 months; (e) if the current dose is 20 mg every 2 months and the AT level is 15-35%, fitusiran is administered at the current dose; (f) if the current dose is 20 mg every 2 months and the AT level is >35% after 6 months, fitusiran is administered at a modified dose of 20 mg every month; (g) if the current dose is 10 mg every 2 months and the AT level is <15%, fitusiran administration is discontinued; (h) if the current dose is 10 mg every 2 months and the AT level is 15-35%, fitusiran is administered at the current dose; or (i) if the current dose is 10 mg every 2 months and the AT level is >35% after 6 months, fitusiran is administered at a modified dose of 10 mg every month. In some embodiments, the fitusiran is in a pharmaceutically acceptable salt form. In some embodiments, the fitusiran is in a sodium salt form.

The present disclosure also provides a method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising: (1) (a) administering to the human patient fitusiran at a starting dose of 50 mg about once every 2 months; and (b) performing one of the following steps: (i) if an antithrombin (AT) level in the human patient is 15-35%, subcutaneously administering to the human patient fitusiran at a dose of 50 mg about once every 2 months, (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (1)(a), after step (1)(a), and after any fitusiran dose modification; (2) (a) administering to the human patient fitusiran at a dose of 20 mg about once every 2 months; and (b) performing one of the following steps: (i) if an AT level in the human patient is 15-35%, subcutaneously administering to the human patient fitusiran at a dose of 20 mg about once every 2 months, (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (2)(a), after step (2)(a), and after any fitusiran dose modification; or (3) (a) after administering to the human patient fitusiran at a dose of 10 mg about once every 2 months; and (b) performing one of the following steps: (i) if an AT level in the human patient is 15-35%, subcutaneously administering to the human patient fitusiran at a dose of 10 mg about once every 2 months, (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (3)(a), after step (3)(a), and after any fitusiran dose modification. In some embodiments, the fitusiran is in a pharmaceutically acceptable salt form. In some embodiments, the fitusiran is in a sodium salt form.

The present disclosure also provides a method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 50 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

The present disclosure also provides a method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 20 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

The present disclosure also provides a method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 10 mg about once every 2 months; (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

In some embodiments, the risk of hepatotoxicity comprises a risk of alanine transaminase (ALT) elevation more than three times the upper limit of normal (ULN), aspartate aminotransferase (AST) elevation more than three times the ULN, severe liver toxicity, liver failure, and/or jaundice. In some embodiments, reducing the risk of hepatotoxicity comprises reducing the occurrence in the human patient of alanine transaminase (ALT) elevation more than three times the upper limit of normal (ULN), aspartate aminotransferase (AST) elevation more than three times the ULN, severe liver toxicity, liver failure, and/or jaundice.

The present disclosure also provides a method of reducing a risk of gallbladder disease in a human patient with hemophilia A or B with or without factor VIII or factor IX inhibitors who receives fitusiran for routine prophylaxis to prevent or reduce the frequency of bleeding episodes, comprising: (a) obtaining a measurement of an antithrombin (AT) level in the human patient, wherein the human patient has previously received fitusiran at a starting dose of 50 mg about once every 2 months; (b) performing one of the following steps: (i) if the AT level is 15-35%, subcutaneously administering to the human patient fitusiran at a dose of 50 mg about once every 2 months, (ii) if the AT level is >35% after 6 months, subcutaneously administering to the human patient fitusiran at a dose of 50 mg about once every month, or (iii) if the AT level is <15%, subcutaneously administering to the human patient fitusiran at a dose of 20 mg about once every 2 months; (c) after administering to the human patient fitusiran at a dose of 20 mg about once every 2 months, obtaining a measurement of an antithrombin (AT) level in the human patient; (d) performing one of the following steps: (i) if the AT level is 15-35%, subcutaneously administering to the human patient fitusiran at a dose of 20 mg about once every 2 months, (ii) if the AT level is >35% after 6 months, subcutaneously administering to the human patient fitusiran at a dose of 20 mg about once every month, or (iii) if the AT level is <15%, subcutaneously administering to the human patient fitusiran at a dose of 10 mg about once every 2 months; (e) after administering to the human patient fitusiran at a dose of 10 mg about once every 2 months, obtaining a measurement of an antithrombin (AT) level in the human patient; and (f) performing one of the following steps: (i) if the AT level is 15-35%, subcutaneously administering to the human patient fitusiran at a dose of 10 mg about once every 2 months, (ii) if the AT level is >35% after 6 months, subcutaneously administering to the human patient fitusiran at a dose of 10 mg about once every month, or (iii) if the AT level is <15%, discontinuing or pausing fitusiran treatment. The present disclosure also provides a method of reducing a risk of gallbladder disease in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 20 mg about once every 2 months; (b) obtaining a measurement of an antithrombin (AT) level in the human patient; and (c) performing one of the following steps: (i) if the AT level is 15-35%, repeating step (a), (ii) if the AT level is >35% after 6 months, subcutaneously administering to the human patient fitusiran at a dose of 20 mg about once every month, or (iii) if the AT level is <15%, subcutaneously administering to the human patient fitusiran at a lower dose amount. In some embodiments, step (c)(iii) comprises subcutaneously administering to the human patient fitusiran at a dose of 10 mg about once every 2 months. In some embodiments, the fitusiran is in a pharmaceutically acceptable salt form. In some embodiments, the fitusiran is in a sodium salt form.

The present disclosure also provides a method of reducing a risk of gallbladder disease in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 10 mg about once every 2 months; (b) obtaining a measurement of an antithrombin (AT) level in the human patient; and (c) performing one of the following steps: (i) if the AT level is 15-35%, repeating step (a), (ii) if the AT level is >35% after 6 months, subcutaneously administering to the human patient fitusiran at a dose of 10 mg about once every month, or (iii) if the AT level is <15%, discontinuing or pausing fitusiran treatment. In some embodiments, the fitusiran is in a pharmaceutically acceptable salt form. In some embodiments, the fitusiran is in a sodium salt form.

The present disclosure also provides a method of reducing a risk of gallbladder disease in a human patient with hemophilia A or B with or without factor VIII or factor IX inhibitors who receives fitusiran for routine prophylaxis to prevent or reduce the frequency of bleeding episodes, comprising subcutaneously administering to the human patient in need thereof fitusiran at a dose of: about 50 mg about once a month or about once every four weeks, about 20 mg about once a month or about once every four weeks, about 10 mg about once a month or about once every four weeks, about 50 mg about once every other month or about once every eight weeks, about 20 mg about once every other month or about once every eight weeks, or about 10 mg about once every other month or about once every eight weeks. In some embodiments, the fitusiran is in a pharmaceutically acceptable salt form. In some embodiments, the fitusiran is in a sodium salt form.

The present disclosure also provides a method of reducing a risk of gallbladder disease in a human patient with hemophilia A or B with or without factor VIII or factor IX inhibitors who receives fitusiran at a current dose for routine prophylaxis to prevent or reduce the frequency of bleeding episodes, the method comprising administering fitusiran at the current dose or a modified dose based on an antithrombin (AT) activity level in the human patient, wherein: (a) if the current dose is 50 mg every 2 months and the AT level is <15%, fitusiran is administered at a modified dose of 20 mg every 2 months; (b) if the current dose is 50 mg every 2 months and the AT level is 15-35%, fitusiran is administered at the current dose; (c) if the current dose is 50 mg every 2 months and the AT level is >35% after 6 months, fitusiran is administered at a modified dose of 50 mg every month; (d) if the current dose is 20 mg every 2 months and the AT level is <15%, fitusiran is administered at a modified dose of 10 mg every 2 months; (e) if the current dose is 20 mg every 2 months and the AT level is 15-35%, fitusiran is administered at the current dose; (f) if the current dose is 20 mg every 2 months and the AT level is >35% after 6 months, fitusiran is administered at a modified dose of 20 mg every month; (g) if the current dose is 10 mg every 2 months and the AT level is <15%, fitusiran administration is discontinued; (h) if the current dose is 10 mg every 2 months and the AT level is 15-35%, fitusiran is administered at the current dose; or (i) if the current dose is 10 mg every 2 months and the AT level is >35% after 6 months, fitusiran is administered at a modified dose of 10 mg every month. In some embodiments, the fitusiran is in a pharmaceutically acceptable salt form. In some embodiments, the fitusiran is in a sodium salt form.

The present disclosure also provides a method of reducing a risk of gallbladder disease in a human patient with hemophilia A or B with or without factor VIII or factor IX inhibitors who receives fitusiran for routine prophylaxis to prevent or reduce the frequency of bleeding episodes, comprising: (1) (a) administering to the human patient fitusiran at a starting dose of 50 mg about once every 2 months; and (b) performing one of the following steps: (i) if an antithrombin (AT) level in the human patient is 15-35%, subcutaneously administering to the human patient fitusiran at a dose of 50 mg about once every 2 months, (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (1)(a), after step (1)(a), and after any fitusiran dose modification; (2) (a) administering to the human patient fitusiran at a dose of 20 mg about once every 2 months; and (b) performing one of the following steps: (i) if an AT level in the human patient is 15-35%, subcutaneously administering to the human patient fitusiran at a dose of 20 mg about once every 2 months, (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (2)(a), after step (2)(a), and after any fitusiran dose modification; or (3) (a) after administering to the human patient fitusiran at a dose of 10 mg about once every 2 months; and (b) performing one of the following steps: (i) if an AT level in the human patient is 15-35%, subcutaneously administering to the human patient fitusiran at a dose of 10 mg about once every 2 months, (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (3)(a), after step (3)(a), and after any fitusiran dose modification. In some embodiments, the fitusiran is in a pharmaceutically acceptable salt form. In some embodiments, the fitusiran is in a sodium salt form.

The present disclosure also provides a method of reducing a risk of gallbladder disease in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 50 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

The present disclosure also provides a method of reducing a risk of gallbladder disease in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 20 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

The present disclosure also provides a method of reducing a risk of gallbladder disease in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 10 mg about once every 2 months; (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

In some embodiments, the risk of gallbladder disease comprises a risk of cholecystitis, cholelithiasis, or a need for cholecystectomy. In some embodiments, reducing the risk of gallbladder disease comprises reducing the occurrence in the human patient of cholecystitis, cholelithiasis, or a need for cholecystectomy. In some embodiments, the gallbladder disease is acute and recurrent gallbladder disease.

The present disclosure also provides a method of reducing thrombotic risk in a human patient with hemophilia A or B with or without factor VIII or factor IX inhibitors who receives fitusiran for routine prophylaxis to prevent or reduce the frequency of bleeding episodes, comprising: (a) obtaining a measurement of an antithrombin (AT) level in the human patient, wherein the human patient has previously received fitusiran at a starting dose of 50 mg about once every 2 months; (b) performing one of the following steps: (i) if the AT level is 15-35%, subcutaneously administering to the human patient fitusiran at a dose of 50 mg about once every 2 months, (ii) if the AT level is >35% after 6 months, subcutaneously administering to the human patient fitusiran at a dose of 50 mg about once every month, or (iii) if the AT level is <15%, subcutaneously administering to the human patient fitusiran at a dose of 20 mg about once every 2 months; (c) after administering to the human patient fitusiran at a dose of 20 mg about once every 2 months, obtaining a measurement of an antithrombin (AT) level in the human patient; (d) performing one of the following steps: (i) if the AT level is 15-35%, subcutaneously administering to the human patient fitusiran at a dose of 20 mg about once every 2 months, (ii) if the AT level is >35% after 6 months, subcutaneously administering to the human patient fitusiran at a dose of 20 mg about once every month, or (iii) if the AT level is <15%, subcutaneously administering to the human patient fitusiran at a dose of 10 mg about once every 2 months; (e) after administering to the human patient fitusiran at a dose of 10 mg about once every 2 months, obtaining a measurement of an antithrombin (AT) level in the human patient; and (f) performing one of the following steps: (i) if the AT level is 15-35%, subcutaneously administering to the human patient fitusiran at a dose of 10 mg about once every 2 months, (ii) if the AT level is >35% after 6 months, subcutaneously administering to the human patient fitusiran at a dose of 10 mg about once every month, or (iii) if the AT level is <15%, discontinuing or pausing fitusiran treatment. In some embodiments, the fitusiran is in a pharmaceutically acceptable salt form. In some embodiments, the fitusiran is in a sodium salt form.

The present disclosure also provides a method of reducing thrombotic risk in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 20 mg about once every 2 months; (b) obtaining a measurement of an antithrombin (AT) level in the human patient; and (c) performing one of the following steps: (i) if the AT level is 15-35%, repeating step (a), (ii) if the AT level is >35% after 6 months, subcutaneously administering to the human patient fitusiran at a dose of 20 mg about once every month, or (iii) if the AT level is <15%, subcutaneously administering to the human patient fitusiran at a lower dose amount. In some embodiments, step (c)(iii) comprises subcutaneously administering to the human patient fitusiran at a dose of 10 mg about once every 2 months. In some embodiments, the fitusiran is in a pharmaceutically acceptable salt form. In some embodiments, the fitusiran is in a sodium salt form.

The present disclosure also provides a method of reducing thrombotic risk in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 10 mg about once every 2 months; (b) obtaining a measurement of an antithrombin (AT) level in the human patient; and (c) performing one of the following steps: (i) if the AT level is 15-35%, repeating step (a), (ii) if the AT level is >35% after 6 months, subcutaneously administering to the human patient fitusiran at a dose of 10 mg about once every month, or (iii) if the AT level is <15%, discontinuing or pausing fitusiran treatment. The present disclosure also provides a method of reducing thrombotic risk in a human patient with hemophilia A or B with or without factor VIII or factor IX inhibitors who receives fitusiran for routine prophylaxis to prevent or reduce the frequency of bleeding episodes, comprising subcutaneously administering to the human patient in need thereof fitusiran at a dose of: about 50 mg about once a month or about once every four weeks, about 20 mg about once a month or about once every four weeks, about 10 mg about once a month or about once every four weeks, about 50 mg about once every other month or about once every eight weeks, about 20 mg about once every other month or about once every eight weeks, or about 10 mg about once every other month or about once every eight weeks. The present disclosure also provides a method of reducing thrombotic risk in a human patient with hemophilia A or B with or without factor VIII or factor IX inhibitors who receives fitusiran at a current dose for routine prophylaxis to prevent or reduce the frequency of bleeding episodes, the method comprising administering fitusiran at the current dose or a modified dose based on an antithrombin (AT) activity level in the human patient, wherein: (a) if the current dose is 50 mg every 2 months and the AT level is <15%, fitusiran is administered at a modified dose of 20 mg every 2 months; (b) if the current dose is 50 mg every 2 months and the AT level is 15-35%, fitusiran is administered at the current dose; (c) if the current dose is 50 mg every 2 months and the AT level is >35% after 6 months, fitusiran is administered at a modified dose of 50 mg every month; (d) if the current dose is 20 mg every 2 months and the AT level is <15%, fitusiran is administered at a modified dose of 10 mg every 2 months; (e) if the current dose is 20 mg every 2 months and the AT level is 15-35%, fitusiran is administered at the current dose; (f) if the current dose is 20 mg every 2 months and the AT level is >35% after 6 months, fitusiran is administered at a modified dose of 20 mg every month; (g) if the current dose is 10 mg every 2 months and the AT level is <15%, fitusiran administration is discontinued; (h) if the current dose is 10 mg every 2 months and the AT level is 15-35%, fitusiran is administered at the current dose; or (i) if the current dose is 10 mg every 2 months and the AT level is >35% after 6 months, fitusiran is administered at a modified dose of 10 mg every month. In some embodiments, the fitusiran is in a pharmaceutically acceptable salt form. In some embodiments, the fitusiran is in a sodium salt form.

The present disclosure also provides a method of reducing thrombotic risk in a human patient with hemophilia A or B with or without factor VIII or factor IX inhibitors who receives fitusiran for routine prophylaxis to prevent or reduce the frequency of bleeding episodes, comprising: (1) (a) administering to the human patient fitusiran at a starting dose of 50 mg about once every 2 months; and (b) performing one of the following steps: (i) if an antithrombin (AT) level in the human patient is 15-35%, subcutaneously administering to the human patient fitusiran at a dose of 50 mg about once every 2 months, (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (1)(a), after step (1)(a), and after any fitusiran dose modification; (2) (a) administering to the human patient fitusiran at a dose of 20 mg about once every 2 months; and (b) performing one of the following steps: (i) if an AT level in the human patient is 15-35%, subcutaneously administering to the human patient fitusiran at a dose of 20 mg about once every 2 months, (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (2)(a), after step (2)(a), and after any fitusiran dose modification; or (3) (a) after administering to the human patient fitusiran at a dose of 10 mg about once every 2 months; and (b) performing one of the following steps: (i) if an AT level in the human patient is 15-35%, subcutaneously administering to the human patient fitusiran at a dose of 10 mg about once every 2 months, (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (3)(a), after step (3)(a), and after any fitusiran dose modification. In some embodiments, the fitusiran is in a pharmaceutically acceptable salt form. In some embodiments, the fitusiran is in a sodium salt form.

The present disclosure also provides a method of reducing thrombotic risk in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 50 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

The present disclosure also provides a method of reducing thrombotic risk in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 20 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

The present disclosure also provides a method of reducing thrombotic risk in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 10 mg about once every 2 months; (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

In some embodiments, the thrombotic risk comprises a risk of a serious thrombotic event. In some embodiments, the serious thrombotic event comprises cerebrovascular accident, cerebral artery embolism, spinal vascular disorder, atrial thrombosis, cerebral venous sinus thrombosis, thrombosis, thrombosis on papillae of an eye, deep venous thrombosis of an arm, subclavian vein thrombosis, superficial venous thrombosis of an arm, cerebral infarction, embolic stroke, or postoperative venous thrombosis. In some embodiments, reducing thrombotic risk comprises reducing the occurrence in the human patient of cerebrovascular accident, cerebral artery embolism, spinal vascular disorder, atrial thrombosis, cerebral venous sinus thrombosis, thrombosis, thrombosis on papillae of an eye, deep venous thrombosis of an arm, subclavian vein thrombosis, superficial venous thrombosis of an arm, cerebral infarction, embolic stroke, or postoperative venous thrombosis. In some embodiments, the human patient has one or more risk factors for thromboembolism. In some embodiments, the risk factor for thromboembolism is persistent AT activity less than 15%, use of a fixed dose of fitusiran 80 mg monthly, presence of indwelling venous catheters, and post-operative state where bleed management guidelines are not followed.

The present disclosure also provides a method of routine prophylaxis to prevent or reduce the frequency of bleeding episodes in a human patient having hemophilia A or B with or without factor VIII or factor IX inhibitors, comprising: (a) subcutaneously administering to the human patient in need thereof fitusiran at a starting dose of 50 mg every 2 months; (b) obtaining a measurement of an antithrombin (AT) level in the human patient; (c) performing one of the following steps: (i) if the AT level is 15-35%, repeating step (a), (ii) if the AT level is >35% after 6 months, subcutaneously administering to the human patient fitusiran at a dose of 50 mg about once every month, or (iii) if the AT level is <15%, subcutaneously administering to the human patient fitusiran at a dose of 20 mg about once every 2 months; (d) after administering to the human patient fitusiran at a dose of 20 mg every 2 months, obtaining a measurement of an antithrombin (AT) level in the human patient; (e) performing one of the following steps: (i) if the AT level is 15-35%, subcutaneously administering to the human patient fitusiran at a dose of 20 mg about once every 2 months, (ii) if the AT level is >35% after 6 months, subcutaneously administering to the human patient fitusiran at a dose of 20 mg about once every month, or (iii) if the AT level is <15%, subcutaneously administering to the human patient fitusiran at a dose of 10 mg about once every 2 months; (f) after administering to the human patient fitusiran at a dose of 10 mg about once every 2 months, obtaining a measurement of an antithrombin (AT) level in the human patient; and (g) performing one of the following steps: (i) if the AT level is 15-35%, subcutaneously administering to the human patient fitusiran at a dose of 10 mg about once every 2 months, (ii) if the AT level is >35% after 6 months, subcutaneously administering to the human patient fitusiran at a dose of 10 mg about once every month, or (iii) if the AT level is <15%, discontinuing or pausing fitusiran treatment. In some embodiments, the fitusiran is in a pharmaceutically acceptable salt form. In some embodiments, the fitusiran is in a sodium salt form.

The present disclosure also provides a method of routine prophylaxis to prevent or reduce the frequency of bleeding episodes in a human patient having hemophilia A or B with or without factor VIII or factor IX inhibitors, comprising: (a) subcutaneously administering to the human patient in need thereof fitusiran at a dose of 20 mg about once every 2 months; (b) obtaining a measurement of an antithrombin (AT) level in the human patient; and (c) performing one of the following steps: (i) if the AT level is 15-35%, repeating step (a), (ii) if the AT level is >35% after 6 months, subcutaneously administering to the human patient fitusiran at a dose of 20 mg about once every month, or (iii) if the AT level is <15%, subcutaneously administering to the human patient fitusiran at a lower dose amount. In some embodiments, step (c)(iii) comprises subcutaneously administering to the human patient fitusiran at a dose of 10 mg about once every 2 months. The present disclosure also provides a method of routine prophylaxis to prevent or reduce the frequency of bleeding episodes in a human patient having hemophilia A or B with or without factor VIII or factor IX inhibitors, comprising: (a) subcutaneously administering to the human patient in need thereof fitusiran at a dose of 10 mg about once every 2 months; (b) obtaining a measurement of an antithrombin (AT) level in the human patient; and (c) performing one of the following steps: (i) if the AT level is 15-35%, repeating step (a), (ii) if the AT level is >35% after 6 months, subcutaneously administering to the human patient fitusiran at a dose of 10 mg about once every month, or (iii) if the AT level is <15%, discontinuing or pausing fitusiran treatment. In some embodiments, the fitusiran is in a pharmaceutically acceptable salt form. In some embodiments, the fitusiran is in a sodium salt form.

The present disclosure also provides a method of routine prophylaxis to prevent or reduce the frequency of bleeding episodes in a human patient having hemophilia A or B with or without factor VIII or factor IX inhibitors, comprising subcutaneously administering to the human patient in need thereof fitusiran at a dose of: about 50 mg about once a month or about once every four weeks, about 20 mg about once a month or about once every four weeks, about 10 mg about once a month or about once every four weeks, about 50 mg about once every other month or about once every eight weeks, about 20 mg about once every other month or about once every eight weeks, or about 10 mg about once every other month or about once every eight weeks. The present disclosure also provides a method of routine prophylaxis to prevent or reduce the frequency of bleeding episodes in a human patient having hemophilia A or B with or without factor VIII or factor IX inhibitors, the method comprising administering fitusiran at a first dose or a modified dose based on an antithrombin (AT) activity level in the human patient, wherein: (a) if the first dose is 50 mg every 2 months and the AT level is <15%, fitusiran is administered at a modified dose of 20 mg every 2 months; (b) if the first dose is 50 mg every 2 months and the AT level is 15-35%, fitusiran is administered again at the first dose; (c) if the first dose is 50 mg every 2 months and the AT level is >35% after 6 months, fitusiran is administered at a modified dose of 50 mg every month; (d) if the first dose is 20 mg every 2 months and the AT level is <15%, fitusiran is administered at a modified dose of 10 mg every 2 months; (e) if the first dose is 20 mg every 2 months and the AT level is 15-35%, fitusiran is administered again at the first dose; (f) if the first dose is 20 mg every 2 months and the AT level is >35% after 6 months, fitusiran is administered at a modified dose of 20 mg every month; (g) if the first dose is 10 mg every 2 months and the AT level is <15%, fitusiran administration is discontinued; (h) if the first dose is 10 mg every 2 months and the AT level is 15-35%, fitusiran is administered again at the first dose; or (i) if the first dose is 10 mg every 2 months and the AT level is >35% after 6 months, fitusiran is administered at a modified dose of 10 mg every month. In some embodiments, the fitusiran is in a pharmaceutically acceptable salt form. In some embodiments, the fitusiran is in a sodium salt form.

The present disclosure also provides a method of routine prophylaxis to prevent or reduce the frequency of bleeding episodes in a human patient with hemophilia A or B with or without factor VIII or factor IX inhibitors, comprising: (1) (a) administering to the human patient fitusiran at a starting dose of 50 mg about once every 2 months; and (b) performing one of the following steps: (i) if an antithrombin (AT) level in the human patient is 15-35%, subcutaneously administering to the human patient fitusiran at a dose of 50 mg about once every 2 months, (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (1)(a), after step (1)(a), and after any fitusiran dose modification; (2) (a) administering to the human patient fitusiran at a dose of 20 mg about once every 2 months; and (b) performing one of the following steps: (i) if an AT level in the human patient is 15-35%, subcutaneously administering to the human patient fitusiran at a dose of 20 mg about once every 2 months, (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (2)(a), after step (2)(a), and after any fitusiran dose modification; or (3) (a) after administering to the human patient fitusiran at a dose of 10 mg about once every 2 months; and (b) performing one of the following steps: (i) if an AT level in the human patient is 15-35%, subcutaneously administering to the human patient fitusiran at a dose of 10 mg about once every 2 months, (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (3)(a), after step (3)(a), and after any fitusiran dose modification. In some embodiments, the fitusiran is in a pharmaceutically acceptable salt form. In some embodiments, the fitusiran is in a sodium salt form.

The present disclosure also provides a method of routine prophylaxis to prevent or reduce the frequency of bleeding episodes in a human patient with hemophilia A or B with or without factor VIII or factor IX inhibitors, comprising: (a) subcutaneously administering to the human patient in need thereof fitusiran at a dose of 50 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

The present disclosure also provides a method of routine prophylaxis to prevent or reduce the frequency of bleeding episodes in a human patient with hemophilia A or B with or without factor VIII or factor IX inhibitors, comprising: (a) subcutaneously administering to the human patient in need thereof fitusiran at a dose of 20 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

The present disclosure also provides a method of routine prophylaxis to prevent or reduce the frequency of bleeding episodes in a human patient with hemophilia A or B with or without factor VIII or factor IX inhibitors, comprising: (a) subcutaneously administering to the human patient in need thereof fitusiran at a dose of 10 mg about once every 2 months; (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

The present disclosure also provides a method of monitoring an antithrombin level in a human patient with hemophilia A or B with or without factor VIII or factor IX inhibitors who receives fitusiran for routine prophylaxis to prevent or reduce the frequency of bleeding episodes, comprising: (a) obtaining a measurement of an antithrombin (AT) level in the human patient, wherein the human patient has previously received fitusiran at a starting dose of 50 mg about once every 2 months; (b) performing one of the following steps: (i) if the AT level is 15-35%, subcutaneously administering to the human patient fitusiran at a dose of 50 mg about once every 2 months, (ii) if the AT level is >35% after 6 months, subcutaneously administering to the human patient fitusiran at a dose of 50 mg about once every month, or (iii) if the AT level is <15%, subcutaneously administering to the human patient fitusiran at a dose of 20 mg about once every 2 months; (c) after administering to the human patient fitusiran at a dose of 20 mg about once every 2 months, obtaining a measurement of an antithrombin (AT) level in the human patient; (d) performing one of the following steps: (i) if the AT level is 15-35%, subcutaneously administering to the human patient fitusiran at a dose of 20 mg about once every 2 months, (ii) if the AT level is >35% after 6 months, subcutaneously administering to the human patient fitusiran at a dose of 20 mg about once every month, or (iii) if the AT level is <15%, subcutaneously administering to the human patient fitusiran at a dose of 10 mg about once every 2 months; (e) after administering to the human patient fitusiran at a dose of 10 mg about once every 2 months, obtaining a measurement of an antithrombin (AT) level in the human patient; and (f) performing one of the following steps: (i) if the AT level is 15-35%, subcutaneously administering to the human patient fitusiran at a dose of 10 mg about once every 2 months, (ii) if the AT level is >35% after 6 months, subcutaneously administering to the human patient fitusiran at a dose of 10 mg about once every month, or (iii) if the AT level is <15%, discontinuing or pausing fitusiran treatment. The present disclosure also provides a method of monitoring an antithrombin level in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 20 mg about once every 2 months; (b) obtaining a measurement of an antithrombin (AT) level in the human patient; and (c) performing one of the following steps: (i) if the AT level is 15-35%, repeating step (a), (ii) if the AT level is >35% after 6 months, subcutaneously administering to the human patient fitusiran at a dose of 20 mg about once every month, or (iii) if the AT level is <15%, subcutaneously administering to the human patient fitusiran at a lower dose amount. In some embodiments, step (c)(iii) comprises subcutaneously administering to the human patient fitusiran at a dose of 10 mg about once every 2 months. In some embodiments, the fitusiran is in a pharmaceutically acceptable salt form. In some embodiments, the fitusiran is in a sodium salt form.

The present disclosure also provides a method of monitoring an antithrombin level in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 10 mg about once every 2 months; (b) obtaining a measurement of an antithrombin (AT) level in the human patient; and (c) performing one of the following steps: (i) if the AT level is 15-35%, repeating step (a), (ii) if the AT level is >35% after 6 months, subcutaneously administering to the human patient fitusiran at a dose of 10 mg about once every month, or (iii) if the AT level is <15%, discontinuing or pausing fitusiran treatment. In some embodiments, the fitusiran is in a pharmaceutically acceptable salt form. In some embodiments, the fitusiran is in a sodium salt form.

The present disclosure also provides a method of monitoring an antithrombin level in a human patient with hemophilia A or B with or without factor VIII or factor IX inhibitors who receives fitusiran for routine prophylaxis to prevent or reduce the frequency of bleeding episodes, comprising subcutaneously administering to the human patient in need thereof fitusiran at a dose of: about 50 mg about once a month or about once every four weeks, about 20 mg about once a month or about once every four weeks, about 10 mg about once a month or about once every four weeks, about 50 mg about once every other month or about once every eight weeks, about 20 mg about once every other month or about once every eight weeks, or about 10 mg about once every other month or about once every eight weeks. In some embodiments, the fitusiran is in a pharmaceutically acceptable salt form. In some embodiments, the fitusiran is in a sodium salt form.

The present disclosure also provides a method of monitoring an antithrombin level in a human patient with hemophilia A or B with or without factor VIII or factor IX inhibitors who receives fitusiran at a current dose for routine prophylaxis to prevent or reduce the frequency of bleeding episodes, the method comprising administering fitusiran at the current dose or a modified dose based on an antithrombin (AT) activity level in the human patient, wherein: (a) if the current dose is 50 mg every 2 months and the AT level is <15%, fitusiran is administered at a modified dose of 20 mg every 2 months; (b) if the current dose is 50 mg every 2 months and the AT level is 15-35%, fitusiran is administered at the current dose; (c) if the current dose is 50 mg every 2 months and the AT level is >35% after 6 months, fitusiran is administered at a modified dose of 50 mg every month; (d) if the current dose is 20 mg every 2 months and the AT level is <15%, fitusiran is administered at a modified dose of 10 mg every 2 months; (e) if the current dose is 20 mg every 2 months and the AT level is 15-35%, fitusiran is administered at the current dose; (f) if the current dose is 20 mg every 2 months and the AT level is >35% after 6 months, fitusiran is administered at a modified dose of 20 mg every month; (g) if the current dose is 10 mg every 2 months and the AT level is <15%, fitusiran administration is discontinued; (h) if the current dose is 10 mg every 2 months and the AT level is 15-35%, fitusiran is administered at the current dose; or (i) if the current dose is 10 mg every 2 months and the AT level is >35% after 6 months, fitusiran is administered at a modified dose of 10 mg every month. In some embodiments, the fitusiran is in a pharmaceutically acceptable salt form. In some embodiments, the fitusiran is in a sodium salt form.

In some embodiments, the antithrombin level is monitored using a kinetic or chromogenic assay. In some embodiments, the antithrombin level is monitored using a chromogenic assay that quantifies functionally active AT in human citrated plasma based on the inhibition of an excess of factor Xa by AT. In some embodiments, the chromogenic assay is an INNOVANCE™ Antithrombin assay.

The present disclosure also provides a method of reducing annualized bleeding rate in a human patient with hemophilia A or B with or without factor VIII or factor IX inhibitors who receives fitusiran for routine prophylaxis to prevent or reduce the frequency of bleeding episodes, comprising: (a) obtaining a measurement of an antithrombin (AT) level in the human patient, wherein the human patient has previously received fitusiran at a starting dose of 50 mg about once every 2 months; (b) performing one of the following steps: (i) if the AT level is 15-35%, subcutaneously administering to the human patient fitusiran at a dose of 50 mg about once every 2 months, (ii) if the AT level is >35% after 6 months, subcutaneously administering to the human patient fitusiran at a dose of 50 mg about once every month, or (iii) if the AT level is <15%, subcutaneously administering to the human patient fitusiran at a dose of 20 mg about once every 2 months; (c) after administering to the human patient fitusiran at a dose of 20 mg about once every 2 months, obtaining a measurement of an antithrombin (AT) level in the human patient; (d) performing one of the following steps: (i) if the AT level is 15-35%, subcutaneously administering to the human patient fitusiran at a dose of 20 mg about once every 2 months, (ii) if the AT level is >35% after 6 months, subcutaneously administering to the human patient fitusiran at a dose of 20 mg about once every month, or (iii) if the AT level is <15%, subcutaneously administering to the human patient fitusiran at a dose of 10 mg about once every 2 months; (e) after administering to the human patient fitusiran at a dose of 10 mg about once every 2 months, obtaining a measurement of an antithrombin (AT) level in the human patient; and (f) performing one of the following steps: (i) if the AT level is 15-35%, subcutaneously administering to the human patient fitusiran at a dose of 10 mg about once every 2 months, (ii) if the AT level is >35% after 6 months, subcutaneously administering to the human patient fitusiran at a dose of 10 mg about once every month, or (iii) if the AT level is <15%, discontinuing or pausing fitusiran treatment. In some embodiments, the fitusiran is in a pharmaceutically acceptable salt form. In some embodiments, the fitusiran is in a sodium salt form.

The present disclosure also provides a method of reducing annualized bleeding rate in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 20 mg about once every 2 months; (b) obtaining a measurement of an antithrombin (AT) level in the human patient; and (c) performing one of the following steps: (i) if the AT level is 15-35%, repeating step (a), (ii) if the AT level is >35% after 6 months, subcutaneously administering to the human patient fitusiran at a dose of 20 mg about once every month, or (iii) if the AT level is <15%, subcutaneously administering to the human patient fitusiran at a lower dose amount. In some embodiments, step (c)(iii) comprises subcutaneously administering to the human patient fitusiran at a dose of 10 mg about once every 2 months. The present disclosure also provides a method of reducing annualized bleeding rate in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 10 mg about once every 2 months; (b) obtaining a measurement of an antithrombin (AT) level in the human patient; and (c) performing one of the following steps: (i) if the AT level is 15-35%, repeating step (a), (ii) if the AT level is >35% after 6 months, subcutaneously administering to the human patient fitusiran at a dose of 10 mg about once every month, or (iii) if the AT level is <15%, discontinuing or pausing fitusiran treatment. In some embodiments, the fitusiran is in a pharmaceutically acceptable salt form. In some embodiments, the fitusiran is in a sodium salt form.

The present disclosure also provides a method of reducing annualized bleeding rate in a human patient with hemophilia A or B with or without factor VIII or factor IX inhibitors who receives fitusiran for routine prophylaxis to prevent or reduce the frequency of bleeding episodes, comprising subcutaneously administering to the human patient in need thereof fitusiran at a dose of: about 50 mg about once a month or about once every four weeks, about 20 mg about once a month or about once every four weeks, about 10 mg about once a month or about once every four weeks, about 50 mg about once every other month or about once every eight weeks, about 20 mg about once every other month or about once every eight weeks, or about 10 mg about once every other month or about once every eight weeks. The present disclosure also provides a method of reducing annualized bleeding rate in a human patient with hemophilia A or B with or without factor VIII or factor IX inhibitors who receives fitusiran at a current dose for routine prophylaxis to prevent or reduce the frequency of bleeding episodes, the method comprising administering fitusiran at the current dose or a modified dose based on an antithrombin (AT) activity level in the human patient, wherein: (a) if the current dose is 50 mg every 2 months and the AT level is <15%, fitusiran is administered at a modified dose of 20 mg every 2 months; (b) if the current dose is 50 mg every 2 months and the AT level is 15-35%, fitusiran is administered at the current dose; (c) if the current dose is 50 mg every 2 months and the AT level is >35% after 6 months, fitusiran is administered at a modified dose of 50 mg every month; (d) if the current dose is 20 mg every 2 months and the AT level is <15%, fitusiran is administered at a modified dose of 10 mg every 2 months; (e) if the current dose is 20 mg every 2 months and the AT level is 15-35%, fitusiran is administered at the current dose; (f) if the current dose is 20 mg every 2 months and the AT level is >35% after 6 months, fitusiran is administered at a modified dose of 20 mg every month; (g) if the current dose is 10 mg every 2 months and the AT level is <15%, fitusiran administration is discontinued; (h) if the current dose is 10 mg every 2 months and the AT level is 15-35%, fitusiran is administered at the current dose; or (i) if the current dose is 10 mg every 2 months and the AT level is >35% after 6 months, fitusiran is administered at a modified dose of 10 mg every month. In some embodiments, the fitusiran is in a pharmaceutically acceptable salt form. In some embodiments, the fitusiran is in a sodium salt form.

The present disclosure also provides a method of reducing annualized bleeding rate in a human patient with hemophilia A or B with or without factor VIII or factor IX inhibitors who receives fitusiran for routine prophylaxis to prevent or reduce the frequency of bleeding episodes, comprising: (1) (a) administering to the human patient fitusiran at a starting dose of 50 mg about once every 2 months; and (b) performing one of the following steps: (i) if an antithrombin (AT) level in the human patient is 15-35%, subcutaneously administering to the human patient fitusiran at a dose of 50 mg about once every 2 months, (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (1)(a), after step (1)(a), and after any fitusiran dose modification; (2) (a) administering to the human patient fitusiran at a dose of 20 mg about once every 2 months; and (b) performing one of the following steps: (i) if an AT level in the human patient is 15-35%, subcutaneously administering to the human patient fitusiran at a dose of 20 mg about once every 2 months, (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (2)(a), after step (2)(a), and after any fitusiran dose modification; or (3) (a) after administering to the human patient fitusiran at a dose of 10 mg about once every 2 months; and (b) performing one of the following steps: (i) if an AT level in the human patient is 15-35%, subcutaneously administering to the human patient fitusiran at a dose of 10 mg about once every 2 months, (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (3)(a), after step (3)(a), and after any fitusiran dose modification. In some embodiments, the fitusiran is in a pharmaceutically acceptable salt form. In some embodiments, the fitusiran is in a sodium salt form.

The present disclosure also provides a method of reducing annualized bleeding rate in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 50 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

The present disclosure also provides a method of reducing annualized bleeding rate in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 20 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

The present disclosure also provides a method of reducing annualized bleeding rate in a human patient with hemophilia A or B with or without factor VIII or factor IX inhibitors who receives fitusiran for routine prophylaxis to prevent or reduce the frequency of bleeding episodes, comprising: (a) subcutaneously administering to the human patient in need thereof fitusiran at a dose of 10 mg about once every 2 months; (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (a), after step (a), and after any fitusiran dose modification. In some embodiments, the fitusiran is in a pharmaceutically acceptable salt form. In some embodiments, the fitusiran is in a sodium salt form.

In some embodiments, the method further comprises administering an effective amount of a replacement factor or bypassing agent (BPA) to treat a bleeding episode that occurs eight or more days after the first fitusiran dose, wherein the effective amount of the replacement factor or BPA is reduced as compared to the recommended effective amount of the replacement factor or BPA for human patients who are not on fitusiran therapy. In some embodiments, the effective amount of the replacement factor or BPA is reduced to one half of a weight-based dose of the recommended effective amount of the replacement factor or BPA for human patients who are not on fitusiran therapy, administered at double the dosing frequency recommended for human patients who are not on fitusiran therapy.

In some embodiments, the human patient is a hemophilia A patient without factor VIII inhibitors. In some embodiments, the replacement factor is factor VIII (FVIII) and a single dose of FVIII is no more than 20 IU/kg. In some embodiments, the single dose of FVIII is 10 IU/kg. In some embodiments, the FVIII administration is repeated, if needed, in no less than 24 hours. In some embodiments, the human patient is a hemophilia B patient without factor IX inhibitors. In some embodiments, the replacement factor is factor IX (FIX) and a single dose of FIX is no more than 30 IU/kg. In some embodiments, the single dose of FIX is 20 IU/kg. In some embodiments, the Factor IX administration is repeated, if needed, in no less than 24 hours for standard half-life FIX or in no less than 5-7 days for extended half-life FIX. In some embodiments, the human patient is a hemophilia A patient with factor VIII inhibitors. In some embodiments, the human patient is a hemophilia B patient with factor IX inhibitors. In some embodiments, the BPA is activated prothrombin complex concentrate (aPCC) and a single dose of aPCC is no more than 50 U/kg. In some embodiments, the single dose of aPCC is 30 U/kg. In some embodiments, the aPCC administration is repeated, if needed, in no less than 24 hours. In some embodiments, the BPA is recombinant factor VIIa (rFVIIa) and a single dose of rFVIIa is no more than 45 g/kg. In some embodiments, the rFVIIa administration is repeated, if needed, in no less than two hours.

In some embodiments, the method further comprises administering an effective amount of a replacement factor or bypassing agent (BPA) to treat a bleeding episode that occurs seven or fewer days after the first fitusiran dose, wherein the effective amount of the replacement factor or BPA is administered according to the human patient's prior dosing regimen of replacement factor or BPA. In some embodiments, the human patient is a hemophilia A patient without factor VIII inhibitors. In some embodiments, the human patient is a hemophilia B patient without factor IX inhibitors. In some embodiments, the human patient is a hemophilia A patient with factor VIII inhibitors. In some embodiments, the human patient is a hemophilia B patient with factor IX inhibitors. In some embodiments, the bleeding episode is a major surgery. In some embodiments, the human patient is a patient not being treated with AT replacement therapy before, during, or after surgery. In some embodiments, the major surgery is an opening into a major body cavity, operation on a joint, removal of an organ, operative alteration of normal anatomy, crossing of a mesenchymal barrier, dental extraction of molar teeth or >3 nonmolar teeth, or tooth implantation. In some embodiments, the human patient has been on prophylactic treatment with a replacement factor or a bypassing agent (BPA), and wherein the method comprises terminating the prophylactic replacement factor or BPA treatment in the human patient within about two weeks or about 14 days or about one week or about seven days of the first dose of fitusiran.

In some embodiments, the method further comprises obtaining a measurement of AT level in the human patient about every four weeks, about every eight weeks, about every month, about every two months, about every four months, about every six months, or about every 12 months. In some embodiments, the method further comprises obtaining a measurement of AT level in the human patient at four weeks or one month, twelve weeks or three months, 20 weeks or five months, and 24 weeks or six months following administration of the starting dose or following administration of a modified dose as compared to the prior dose. In some embodiments, the method further comprises obtaining a second measurement of AT level if the AT level is <15%. In some embodiments, the method further comprises subcutaneously administering to the human patient fitusiran at a lower dose amount 3 months after the prior dose if the second measurement of AT level is <15%. In some embodiments, the method further comprises discontinuing or pausing fitusiran treatment if the second measurement of AT level is <15%. In some embodiments, the method further comprises obtaining a measurement of AT level at four weeks or one month, twelve weeks or three months, 20 weeks or five months, and 24 weeks or six months after a dose reduction in fitusiran. In some embodiments, the method further comprises administering to the human patient fitusiran at a higher dose amount if the AT level is >35% after six months. In some embodiments, the method further comprises administering to the human patient fitusiran at a higher dose amount if the human patient has not achieved satisfactory bleed control. In some embodiments, the method further comprises obtaining a measurement of AT level at four weeks or one month, twelve weeks or three months, 20 weeks or five months, and 24 weeks or six months after a dose escalation in fitusiran. In some embodiments, the method further comprises obtaining a subsequent measurement of AT level about every 12 months if the AT level is 15-35%. In some embodiments, the obtaining a measurement of the AT level in the human patient comprises use of a kinetic or chromogenic assay. In some embodiments, obtaining a measurement of the AT level in the human patient comprises use of a chromogenic assay that quantifies functionally active AT in human citrated plasma based on the inhibition of an excess of factor Xa by AT. In some embodiments, obtaining a measurement of the AT level in the human patient comprises use of an INNOVANCE™ Antithrombin assay.

In some embodiments, the method further comprises subcutaneously administering fitusiran to the human patient at a dose amount and a dosing frequency sufficient to maintain the AT level in the human patient at 15-35%. In some embodiments, the method further comprises subcutaneously administering fitusiran at: 10 mg QM or Q4W, 10 mg Q2M or Q8W, 20 mg QM or Q4W, 20 mg Q2M or Q8W, 50 mg QM or Q4W, or 50 mg Q2M or Q8W. In some embodiments, the method further comprises obtaining a liver function test in the human patient (i) prior to administering the starting dose of fitusiran to determine a baseline level of liver function; and (ii) about every month following initiation of fitusiran treatment. In some embodiments, the liver function test is an aspartate transaminase test, an alanine aminotransferase test, and/or a measurement of total bilirubin. In some embodiments, the liver function test is obtained about every month for at least the first six months following initiation of fitusiran treatment. In some embodiments, the method further comprises discontinuing or pausing fitusiran treatment if the aspartate transaminase or alanine aminotransferase test shows aspartate transaminase or alanine aminotransferase levels that are progressing or are persistent and five times the upper limit of normal or if the total bilirubin is >2.5 mg/dL. In some embodiments, fitusiran treatment is resumed if the aspartate transaminase or alanine aminotransferase test shows aspartate transaminase or alanine aminotransferase levels have returned to baseline level and if the total bilirubin level has returned to baseline.

In some embodiments, the method further comprises discontinuing or pausing fitusiran treatment if the human patient is diagnosed with acute and recurrent gallbladder disease. In some embodiments, the acute and recurrent gallbladder disease comprises cholecystitis, cholelithiasis, or a need for cholecystectomy. In some embodiments, the human patient does not have (i) clinically significant liver disease, (ii) ALT>1.5×upper limit of normal reference range (ULN), (iii) AST>1.5×upper limit of normal reference range (ULN), (iv) hepatitis C, (v) hepatitis A, (vi) hepatitis E, (vii) hepatitis B, (viii) acute and recurrent gallbladder disease, (ix) symptomatic gallbladder disease, (x) established thrombophilic conditions, (xi) a prior history of thrombosis, (xii) an indwelling venous catheter, (xiii) persistent AT activity <15%, or (xiv) known hepatic impairment. In some embodiments, the known hepatic impairment is Child-Pugh Class A, B, or C. In some embodiments, the human patient is an adult or adolescent patient twelve years or older. In some embodiments, fitusiran is provided in a phosphate-buffered saline (PBS) at about 40-200 mg/mL. In some embodiments, fitusiran is provided in a PBS at about 40 mg/mL or about 100 mg/mL. In some embodiments, the PBS has a pH of 7.

The present disclosure also provides fitusiran for use in any of the preceding methods. The present disclosure also provides use of fitusiran in the manufacture of a medicament for use in any of the preceding methods. The present disclosure also provides a pharmaceutical composition comprising fitusiran for use in any of the preceding methods. The present disclosure also provides an article of manufacture for use in any of the preceding methods. In some embodiments, the article of manufacture is a kit. In some embodiments, the article of manufacture is a container containing one or more doses of fitusiran, each dose being 50 mg, 20 mg, or 10 mg. In some embodiments, the 50 mg of fitusiran is in 0.5 mL of a PBS, the 20 mg of fitusiran is in 0.2 mL of a PBS, the 20 mg of fitusiran is in 0.5 mL of a PBS, the 10 mg of fitusiran is in 0.25 mL of a PBS, or the 10 mg of fitusiran is in 0.1 mL of a PBS. In some embodiments, the PBS has a pH of 7. In some embodiments, the container is a single-use, single-dose prefilled syringe. In some embodiments, the container is a single-use glass vial.

The present disclosure also provides an article of manufacture comprising: (a) a packaging material; (b) fitusiran; and (c) a label or package insert contained within the packaging material indicating that serious thrombotic events can occur. The present disclosure also provides an article of manufacture comprising: (a) a packaging material; (b) fitusiran; and (c) a label or package insert contained within the packaging material indicating that acute and recurrent gallbladder disease can occur. The present disclosure also provides an article of manufacture comprising: (a) a packaging material; (b) fitusiran; and (c) a label or package insert contained within the packaging material indicating that hepatotoxicity can occur. The present disclosure also provides a package comprising fitusiran and a label, said label comprising one or more messages that: (a) serious thrombotic events have been reported; (b) AT levels should be monitored to reduce the potential risk of thrombosis; or (c) fitusiran prophylaxis should be interrupted if clinical symptoms, signs, or imaging findings consistent with a thrombotic event occur, and a thrombotic event should be managed as clinically indicated. The present disclosure also provides a package comprising fitusiran and a label, said label comprising one or more messages that: (a) treatment with fitusiran has been associated with an increased occurrence of acute and recurrent gallbladder disease; or (b) if gallbladder disease occurs, consider interrupting or discontinuing fitusiran treatment. The present disclosure also provides a package comprising fitusiran and a label, said label comprising one or more messages that: (a) hepatotoxicity has been reported; (b) baseline liver function tests should be obtained and liver function should be monitored; (c) use of fitusiran should be avoided in patients with known hepatic impairment; or (d) fitusiran should be permanently discontinued if alanine aminotransferase or aspartate transaminase elevations greater than five times the upper limit of normal reoccur or the patient experiences jaundice thought to be from hepatotoxicity with other causes of liver test elevation ruled out. The present disclosure also provides a package comprising fitusiran and a label, said label comprising a printed statement which informs a reader that the mean Cmax of fitusiran in patients with hemophilia A and B, with or without inhibitors was 34.4 ng/mL (CV 29%) for fitusiran at 20 mg and 84.1 ng/mL (CV 70%) at 50 mg. The present disclosure also provides a package comprising fitusiran and a label, said label comprising a printed statement which informs a reader that the mean AUC of fitusiran in patients with hemophilia A and B, with or without inhibitors was 491 ng h/mL (CV 17%) for fitusiran at 20 mg and 1290 ng h/mL (CV 29%) for fitusiran at 50 mg. The present disclosure also provides a package comprising fitusiran and a label, said label comprising a printed statement which informs a reader that fitusiran has an apparent clearance of 41.9 CL/F (CV 20%) at 20 mg and 50.8 CL/F (CV 140%) at 50 mg.

The present disclosure also provides a method of promoting the use of fitusiran, the method comprising the step of conveying to a recipient at least one message selected from: (a) serious thrombotic events have been reported; (b) AT levels should be monitored to reduce the potential risk of thrombosis; (c) fitusiran prophylaxis should be interrupted if clinical symptoms, signs, or imaging findings consistent with a thrombotic event occur, and a thrombotic event should be managed as clinically indicated; (d) treatment with fitusiran has been associated with an increased occurrence of acute and recurrent gallbladder disease; (e) if gallbladder disease occurs, consider interrupting or discontinuing fitusiran treatment; (f) hepatotoxicity has been reported; (g) baseline liver function tests should be obtained and liver function should be monitored; (h) use of fitusiran should be avoided in patients with known hepatic impairment; (i) fitusiran should be permanently discontinued if alanine aminotransferase or aspartate transaminase elevations greater than five times the upper limit of normal reoccur or the patient experiences jaundice thought to be from hepatotoxicity with other causes of liver test elevation ruled out. The present disclosure also provides a method of providing fitusiran, wherein said fitusiran is provided along with information indicating that: (a) serious thrombotic events have been reported; (b) AT levels should be monitored to reduce the potential risk of thrombosis; (c) fitusiran prophylaxis should be interrupted if clinical symptoms, signs, or imaging findings consistent with a thrombotic event occur, and a thrombotic event should be managed as clinically indicated; (d) treatment with fitusiran has been associated with an increased occurrence of acute and recurrent gallbladder disease; (e) if gallbladder disease occurs, consider interrupting or discontinuing fitusiran treatment; (f) hepatotoxicity has been reported; (g) baseline liver function tests should be obtained and liver function should be monitored; (h) use of fitusiran should be avoided in patients with known hepatic impairment; or (i) fitusiran should be permanently discontinued if alanine aminotransferase or aspartate transaminase elevations greater than five times the upper limit of normal reoccur or the patient experiences jaundice thought to be from hepatotoxicity with other causes of liver test elevation ruled out.

Also provided herein is a pharmaceutical composition, comprising fitusiran at a concentration of about 35 mg/ml to about 45 mg/mL and phosphate buffered saline (PBS) at a concentration of about 1 mM to about 10 mM. Such pharmaceutical compositions have a pH and an osmolality suitable for subcutaneous administration to a subject. In some embodiments, the fitusiran is in a pharmaceutically acceptable salt form. In some embodiments, the fitusiran is in a sodium salt form.

In some embodiments, the concentration of PBS is about 3 to about 6 mM. in some embodiments, the concentration of PBS is about 5 mM. In some embodiments, the concentration of the fitusiran in the pharmaceutical composition is about 40 mg/mL.

In one embodiment, the present invention provides a pharmaceutical composition wherein the composition comprises sodium chloride, dibasic sodium phosphate (heptahydrate), and monobasic sodium phosphate. In some embodiments, the sodium chloride is about 7.68 to 7.69 mg/mL. In some embodiments, the dibasic sodium phosphate (heptahydrate) is about 0.932 to 0.934 mg/mL. In some embodiments, the monobasic sodium phosphate (monohydrate) is about 0.208 to 0.211 mg/mL. In some embodiments, 1 ml of the pharmaceutical composition comprises about 40 mg of the fitusiran, 7.672 mg of the sodium chloride, 0.933 mg of the dibasic sodium phosphate (heptahydrate), and 0.2095 mg of the monobasic sodium phosphate (monohydrate). In some embodiments, 0.5 mL of the pharmaceutical composition comprises about 20 mg of the fitusiran, 3.836 mg of sodium chloride, 0.467 mg of dibasic sodium phosphate, and 0.105 mg of monobasic sodium phosphate.

In some embodiments, the pH of the composition is between about 5.0 to about 8.0. In some embodiments, the pH of the composition is between about 6.0 to about 8.0. In some embodiments, the pH of the composition is between about 6.5 to about 7.5. In some embodiments, the pH of the composition is between about 6.8 to about 7.2. In some embodiments, the pH of the composition is about 7.0. In some embodiments, the osmolality of the composition is between about 50 and about 400 mOsm/kg. In some embodiments, the osmolality of the composition is between about 100 and about 400 mOsm/kg. In some embodiments, the osmolality of the composition is between about 240 and about 390 mOsm/kg. In some embodiments, the osmolality of the composition is about 300 mOsm/kg.

In some embodiments, the composition is stable for up to about 36 months when stored at about 2° C. to about 8° C.

In some embodiments, the composition is stable for up to about 36 months when stored at about 25° C. and 60% relative humidity (RH). In some embodiments, the composition is stable for up to about 6 months when stored at about 40° C. and 75% relative humidity (RH). In some embodiments, the composition comprises not less than (NLT) about 90.5 area % duplex and not more than (NMT) about 5 area % single strands as determined by purity non-denaturing IPRP-HPLC. In some embodiments, the composition comprises not less than (NLT) about 85.0 area % total single strands as determined by purity denaturing AX-HPLC. In some embodiments, the composition comprises not less than (NLT) about 80.0 area % total single strands as determined by purity denaturing IPRP-HPLC. In some embodiments, the composition comprises a density of about 1 mg/mL. In some embodiments, the composition comprises a density of about 1.02364 mg/mL. In some embodiments, the composition comprises a viscosity of about 1.4 pascal-seconds at about 20° C.

In another embodiment, the present invention provides a pharmaceutical composition, comprising fitusiran at a concentration of about 40 mg/mL and phosphate buffered saline (PBS) at a concentration of about 5 mM, wherein the pH of the pharmaceutical composition is about 6.8 to about 7.2, and wherein the osmolality of the pharmaceutical composition is about 300 mOsm/kg. In some embodiments, the fitusiran is in a pharmaceutically acceptable salt form. In some embodiments, the fitusiran is in a sodium salt form.

In another embodiment, the present invention provides a pharmaceutical composition, comprising fitusiran and phosphate-buffered saline (PBS), wherein 1 ml of the pharmaceutical composition comprises about 40 mg of fitusiran, 7.672 mg of sodium chloride, 0.933 mg of dibasic sodium phosphate (heptahydrate), 0.2095 mg of monobasic sodium phosphate (monohydrate), and water for injection, wherein the pH of the pharmaceutical composition is about 6.8 to about 7.2. In another embodiment, the present invention provides a pharmaceutical composition comprising fitusiran and phosphate-buffered saline (PBS), wherein 0.5 mL of the pharmaceutical composition comprises about 20 mg of fitusiran, 3.836 mg of sodium chloride, 0.467 mg of dibasic sodium phosphate, 0.105 mg of monobasic sodium phosphate, and water for injection, wherein the pH of the pharmaceutical composition is about 6.8 to about 7.2. In some embodiments, the fitusiran is in a pharmaceutically acceptable salt form. In some embodiments, the fitusiran is in a sodium salt form. In some embodiments, the pH of the pharmaceutical composition is about 7.0.

In another embodiment, the present invention provides a method of treating hemophilia A or hemophilia B prophylactically in a patient in need thereof, comprising administering to the patient the pharmaceutical composition of any one of the preceding embodiments.

In another embodiment, the present invention provides a use of the pharmaceutical composition of any one of the preceding embodiments in the manufacture of a medicament for prophylactic treatment of hemophilia A or hemophilia B in a patient in need thereof.

In another embodiment, the present invention provides the pharmaceutical composition of any one of the preceding embodiments in the use in the prophylactic treatment of hemophilia A or hemophilia B in a patient in need thereof.

In another embodiment, the present invention provides the method of treating, the use, or the pharmaceutical composition for use, wherein the patient is a hemophilia A patient with inhibitors. In another embodiment, the present invention provides the method of treating, the use, or the pharmaceutical composition for use, wherein the patient is a hemophilia A patient without inhibitors. In another embodiment, the present invention provides the method of treating, the use, or the pharmaceutical composition for use, wherein the patient is a hemophilia B patient with inhibitors. In another embodiment, the present invention provides the method of treating, the use, or the pharmaceutical composition for use, wherein the patient is a hemophilia B patient without inhibitors.

In another embodiment, the present invention provides a method of administering the pharmaceutical composition of any one of the preceding embodiments, comprising subcutaneous injection of the pharmaceutical composition with a prefilled syringe containing the pharmaceutical composition. In another embodiment, the present invention provides a method of administering the pharmaceutical composition of any one of the preceding embodiments, comprising subcutaneous injection of the pharmaceutical composition from a prefilled vial containing the pharmaceutical composition. In another embodiment, the present invention provides an article of manufacture comprising the pharmaceutical composition of any one of the preceding embodiments. In some embodiments, the article of manufacture is a syringe or a vial.

In another embodiment, the present invention provides a vial comprising the pharmaceutical composition of any one of the preceding embodiments. In some embodiments, the vial comprises about 0.5 mL to about 2.0 ml of the pharmaceutical composition. In another embodiment, the present invention provides a syringe comprising the pharmaceutical composition of any one of the preceding embodiments. In some embodiments, the syringe is a 1 ml syringe. In some embodiments, the syringe comprises about 0.5 ml of the pharmaceutical composition. In some embodiments, the syringe comprises 0.535 mL of the pharmaceutical composition. In some embodiments, the syringe is for injection of a 20 mg dose of fitusiran.

In some embodiments, the syringe comprises about 0.25 mL of the pharmaceutical composition. In some embodiments, the syringe is for injection of a 10 mg dose of fitusiran. In some embodiments, the syringe comprises a 29 G needle. In some embodiments, the syringe comprises a 30 G needle. In some embodiments, the syringe is a prefilled syringe. In some embodiments, the prefilled syringe is a prefilled pen.

In another embodiment, the present invention provides a kit comprising the article of manufacture of any one of the preceding embodiments, the syringe of any one of the preceding embodiments, or the vial of any one of the preceding embodiments, and a label or a package insert.

Other features, objectives, and advantages of the invention are apparent in the detailed description that follows. It should be understood, however, that the detailed description, while indicating embodiments of the invention, is given by way of illustration only, not limitation. Various changes and modification within the scope of the invention will become apparent to those skilled in the art from the detailed description.

### BRIEF DESCRIPTION OF THE FIGURES

FIG. 1 shows the expanded structural formula of fitusiran in sodium salt form (also referred to as “fitusiran sodium”), and the molecular formulas and molecular weights of fitusiran and fitusiran sodium, including the sense strand and antisense strand.

FIG. 2 depicts representative excipients and associated function or characteristic for use in the fitusiran formulation.

FIG. 3 depicts the viscosity of the 40 mg/mL fitusiran formulation at varying temperatures (e.g., 5° C. to 30° C.).

FIG. 4 depicts a semi-mechanistic population kinetic-pharmacodynamic model used for simulations of the AT-based dosing regimen described herein.

The contents of all references, patents and published patent applications cited throughout this application, as well as the Figures, are hereby incorporated by reference.

### DETAILED DESCRIPTION OF THE INVENTION

The present disclosure provides methods of using fitusiran for routine prophylaxis to prevent or reduce the frequency of bleeding episodes in patients with hemophilia A (congenital factor VIII deficiency) or hemophilia B (congenital factor IX deficiency), with or without factor VIII or factor IX inhibitors. In some embodiments, the present methods reduce the risk of hepatotoxicity, the risk of gallbladder disease that may be acute and recurrent, the thrombotic risk, or the annualized bleeding rate in a human patient receiving fitusiran. The present disclosure also provides methods of monitoring an antithrombin (AT) level in a human patient receiving fitusiran. In some embodiments, the present methods reduce the dose of fitusiran after obtaining a measurement of an AT level in a human patient receiving fitusiran. In some embodiments, fitusiran is given at a dose of 50 mg, 20 mg, or 10 mg, at a frequency of about once a month or about once every other month. In some embodiments, the patient is an adult or adolescent ≥12 years old.

The present disclosure also provide a method of routine prophylaxis to prevent or reduce the frequency of bleeding episodes in a human patient having hemophilia A or B with or without factor VIII or factor IX inhibitors, wherein the patient is further administered an effective amount of replacement factor (clotting factor concentrates or CFC) or bypassing agent (BPA) to treat a bleeding episode (e.g., breakthrough bleeding or bleeding associated with surgery), wherein the effective amount of the replacement factor or BPA is reduced as compared to the recommended (i.e., standard) effective amount of the replacement factor or BPA used on patients not receiving fitusiran therapy.

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without factor VIII or factor IX inhibitors refers to a patient who does not have such alloantibodies. The present treatment methods are beneficial for hemophilia A patients with or without factor VIII inhibitors, as well as for hemophilia B patients with or without factor IX inhibitors.

The present methods are particularly useful in mitigating side effects that may be associated with using fitusiran for routine prophylaxis, given the need to consider risks associated with persistently elevated or reduced AT activity levels in patients. The present methods integrate the monitoring of AT activity with a dose escalation or de-escalation regimen in response to AT activity levels. For example, in some embodiments, the target AT activity level is 15-35% and the dose of fitusiran is escalated or de-escalated to maintain that target level.

Monitoring AT activity levels and adjusting the dose of fitusiran accordingly can support the efficacy of fitusiran for routine prophylaxis yet reduce the risk of certain side effects, including but not limited to hepatotoxicity, acute and recurrent gallbladder disease, and serious thrombotic events. When AT activity level is persistently low in a patient, for example <15%, the risk of an adverse event such as thrombosis is greater. The present invention includes a dose de-escalation regimen to preserve fitusiran efficacy and safety in such cases, based at least in part on unexpected simulation results indicating that lower doses of fitusiran (e.g., 10 mg Q2M, 10 mg QM) can maintain patients at steady state 15-35% AT levels. Thus, in the present methods, doses as low as 10 mg QM or 10 Q2M are incorporated into a dose de-escalation regimen that is responsive to AT activity levels.

### I. Fitusiran Pharmaceutical Compositions

Hemophilia results in a profound defect in thrombin generation, and further, hemophilia severity is correlated with the inability to generate thrombin. Without being bound by theory, it is believed that fitusiran-mediated lowering of antithrombin (AT) levels will increase thrombin generation and thus improve hemostasis in patients with hemophilia. Antithrombin is encoded by the SERPINC1 gene.

Fitusiran, whose structure is described herein, is a synthetic, chemically modified double-stranded small interfering RNA (siRNA) oligonucleotide covalently linked to a triantennary N-acetyl-galactosamine (GalNAc) ligand targeting the AT3 mRNA in the liver, thereby suppressing the synthesis of antithrombin. The nucleosides in each strand of fitusiran are connected through either 3′-5′ phosphodiester or phosphorothioate linkages, thus forming the sugar-phosphate backbone of the oligonucleotide.

Fitusiran is a double-stranded small interfering ribonucleic acid that targets AT mRNA which has been conjugated to a triantennary N-acetyl galactosamine ligand for directed delivery to the liver. The molecular formula of fitusiran in sodium salt form (also referred to as “fitusiran sodium”) is C₅₂₀H₆₃₆F₂₁N₁₇₅Na₄₃O₃₀₉P₄₃S₆ and the molecular weight of fitusiran sodium is 17,193 Da.

Fitusiran is a double-stranded ribonucleic acid comprising a sense strand and an antisense strand which contain 21 and 23 nucleotides, respectively. The 3′-end of the sense strand is conjugated to the GalNAc containing moiety (referred to as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, and WO 2019/014187.

The two nucleotide strands of fitusiran are shown below:

sense strand:
(SEQ ID NO: 1)
5′Gf-ps-Gm-ps-Uf-Um-Af-Am-Cf-Am-Cf-Cf-Af-Um-Uf-Um-
Af-Cm-Uf-Um-Cf-Am-Af-L96 3′,
and
antisense strand:
(SEQ ID NO: 2)
5′ Um-ps-Uf-ps-Gm-Af-Am-Gf-Um-Af-Am-Af-Um-Gm-Gm-
Uf-Gm-Uf-Um-Af-Am-Cf-Cm-ps-Am-ps-Gm 3′,

wherein
Af=2′-fluoroadenosine (i.e., 2′-deoxy-2′-fluoroadenosine)
Cf=2′-fluorocytidine (i.e., 2′-deoxy-2′-fluorocytidine)
Gf=2′-fluoroguanosine (i.e., 2′-deoxy-2′-fluoroguanosine)
Uf=2′-fluorouridine (i.e., 2′-deoxy-2′-fluorouridine)
Am=2′-O-methyladenosine
Cm=2′-O-methylcytidine
Gm=2′-O-methylguanosine
Um=2′-O-methyluridine
“-” (hyphen)=3′-5′ phosphodiester linkage
“-ps-”=3′-5′ phosphorothioate linkage
and wherein L96 has the following formula:

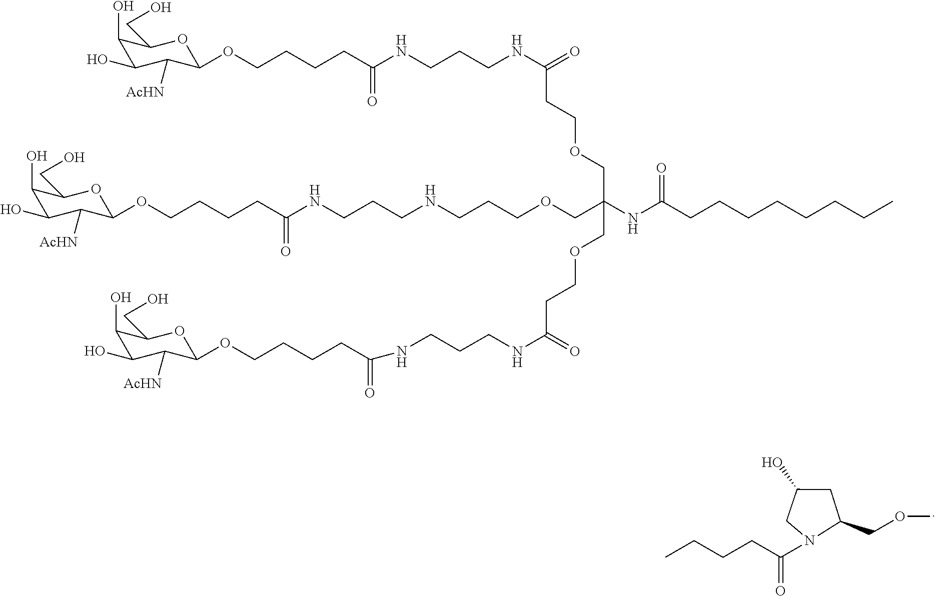

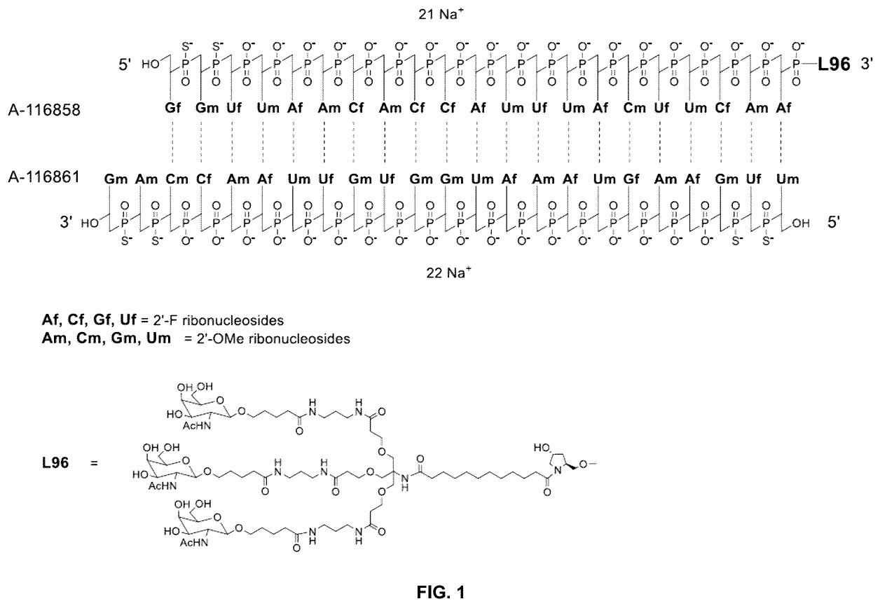

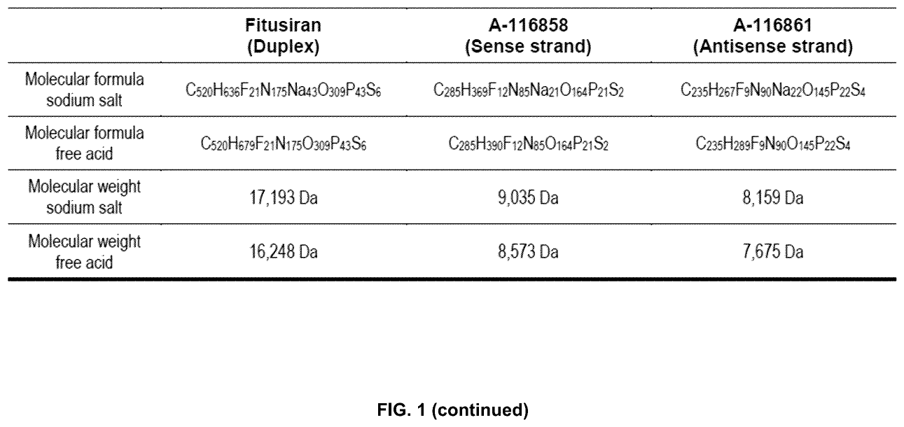

The expanded structural formula of fitusiran sodium and the molecular formulas and molecular weights of fitusiran and fitusiran sodium, including the sense strand and antisense strand are shown in FIG. 1. As used herein, the term 2′-fluoroadenosine is used interchangeably with the term 2′-deoxy-2′-fluoroadenosine; the term 2′-fluorocytidine is used interchangeably with the term 2′-deoxy-2′-fluorocytidine; the term 2′-fluoroguanosine is used interchangeably with the term 2′-deoxy-2′-fluoroguanosine, and the term 2′-fluorouridine is used interchangeably with the term 2′-deoxy-2′-fluorouridine.

The structure of fitusiran can also be described using the following diagram, wherein the X is O:

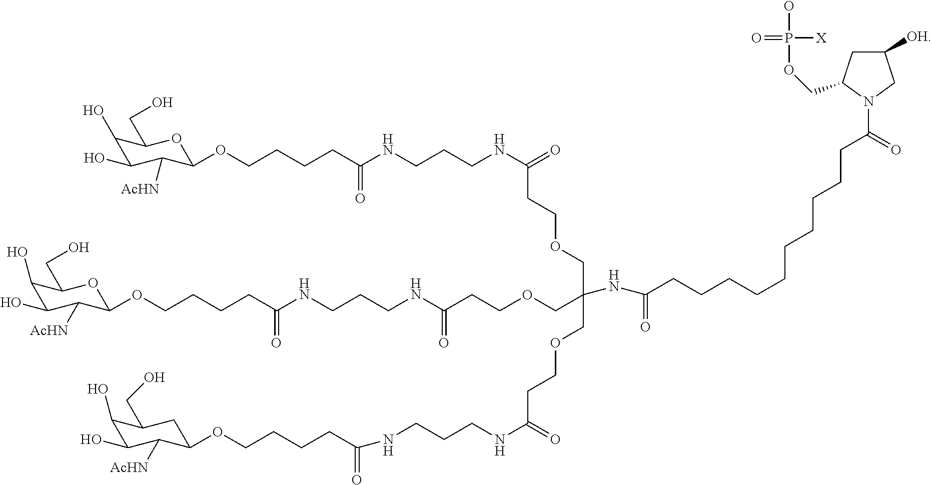

For use in the present treatment methods, fitusiran may be provided in a pharmaceutical composition comprising it and a pharmaceutically acceptable excipient. In some embodiments, fitusiran is in a pharmaceutically acceptable salt form. In certain embodiments, fitusiran is in a sodium salt form.

In some embodiments, fitusiran is provided in an aqueous solution at a concentration of about 1 to about 200 mg/mL (e.g., about 20 to about 150 mg/mL, about 80 to about 110 mg/mL, or about 90 to about 110 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical composition comprises fitusiran at a concentration of about 6.25, about 12.5, about 25, about 40, about 50, about 75, about 100, about 125, about 150, or about 200 mg/mL. In further embodiments, the pharmaceutical composition comprises fitusiran at 40 mg/mL or at 100 mg/mL.

Unless otherwise indicated, a fitusiran weight recited in the present disclosure is the weight of fitusiran free acid (the active moiety), even though fitusiran is injected to patients subcutaneously in a pharmaceutically acceptable salt form, for example, as fitusiran sodium (in an aqueous solution). For example, 100 mg/mL fitusiran means 100 mg of fitusiran free acid (equivalent to 106 mg fitusiran sodium, the drug substance) per mL. In another example, 40 mg/mL fitusiran means 40 mg of fitusiran free acid (equivalent to 42.4 mg fitusiran sodium, the drug substance) per mL.

In some embodiments, the pharmaceutical compositions comprise fitusiran sodium in a phosphate-buffered saline. The phosphate concentration in the solution may be about 1 to 10 mM (e.g., 2, 3, 4, 5, 6, 7, 8, or 9 mM), with a pH of 6.0-8.0. The pharmaceutical compositions herein may include a preservative such as EDTA. Alternatively, the pharmaceutical compositions are preservative-free. In particular embodiments, the pharmaceutical composition is preservative-free and comprises, consists of, or consists essentially of about 100 mg of fitusiran per mL of a 5 mM phosphate buffered saline (PBS) solution. In particular embodiments, the pharmaceutical composition is preservative-free and comprises, consists of, or consists essentially of about 40 mg of fitusiran per mL of a 5 mM PBS solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the composition to 7.0.

The pharmaceutical composition may be provided in a container (e.g., a vial or a syringe). The container may contain single or multiple doses. In some embodiments, the container is a single-use container (e.g., a single-use ampule or a single-use syringe such as a single-use prefilled syringe), with each container containing about 10 to about 100 mg fitusiran (e.g., about 10 mg, about 20 mg, about 25 mg, about 40 mg, or about 50 mg). In some embodiments, a Luer Lock syringe is used to withdraw the pharmaceutical composition from the single-use container. In some embodiments, the single-use container is a prefilled pen (PFP). The fitusiran sodium may be provided in a solid form in the container and reconstituted in an aqueous solution (e.g., PBS) prior to use, with the reconstituted solution containing about 1 to about 150 mg/mL (e.g., 40 mg/mL or 100 mg/mL) fitusiran. In some embodiments, fitusiran is provided in sodium salt form in a single-use glass vial or a single-use prefilled syringe (e.g., one with a safety system). In further embodiments, each vial or syringe contains about 50 mg of fitusiran in about 0.5 mL, about 20 mg of fitusiran in about 0.2 mL, about 20 mg of fitusiran in about 0.5 mL, about 10 mg of fitusiran in about 0.25 mL, or about 10 mg of fitusiran in about 0.1 mL of 5 mM phosphate buffered saline solution (pH 7.0); and the solution is administered to patients through subcutaneous injection. The solution can be stored at 2 to 30° C. (e.g., 2 to 8° C.).

In particular embodiments, the pharmaceutical composition for subcutaneous injection contains fitusiran in a 5 mM phosphate buffered saline having 0.64 mM NaH₂PO₄, 4.36 mM Na₂HPO₄, and 84 mM NaCl at pH 7.0. In certain embodiments, the pharmaceutical composition for subcutaneous injection is shown in Table 1A.

TABLE 1A
Exemplary Formulation

| | Composition | | | |
|---|---|---|---|---|
| | | | Per unit | Per unit |
| | Percentage | Per ml | (vial) | (syringe) |
| Components | [%] | [mg] | [mg] | [mg] |
| Fitusiran (active moiety) | 10 | 100 [106] | 20 [21.2] | 50 [53.0] |
| [equivalent to fitusiran sodium] | | | | |
| Sodium chloride | 0.49 | 4.909 | 0.982 | 2.455 |
| Dibasic sodium phosphate | 0.12 | 1.169 | 0.234 | 0.585 |
| (heptahydrate) | | | | |
| Monobasic sodium phosphate | <0.01 | 0.0885 | 0.018 | 0.044 |
| (monohydrate) | | | | |
| Phosphoric acid, concentrated | — | q.s. pH 7.0 | q.s. pH 7.0 | q.s. pH 7.0 |
| Sodium hydroxide | — | q.s. pH 7.0 | q.s. pH 7.0 | q.s. pH 7.0 |
| Water for injection | q.s. 100 | q.s. 1 mL | q.s. 0.2 mL | q.s. 0.5 mL |

q.s.: quantum satis.

In some embodiments, fitusiran is packaged in a prefilled pen containing 50 mg of fitusiran (equivalent to 53 mg fitusiran sodium). In some embodiments, each single-use prefilled pen contains 50 mg of fitusiran in 0.5 mL solution at a concentration of 100 mg/mL. In some embodiments, each prefilled pen is designed to deliver 50 mg fitusiran in 0.5 mL which also contains: sodium chloride (2.455 mg), dibasic sodium phosphate (0.585 mg), monobasic sodium phosphate (0.044 mg), phosphoric acid (q.s. pH 7.0), sodium hydroxide (q.s. pH 7.0), and water for injection (q.s. 0.5 mL). Fitusiran may be supplied as a sterile, clear solution for subcutaneous injection in the 50 mg PFP. In some embodiments, the PFP contains a single dose of fitusiran and comprises a prefilled syringe (PFS) assembled with an automated injection system (pen). The PFS is a 1 mL colorless type 1 glass syringe with a 29 gauge, (%/2 inch) stainless steel staked needle covered with a rigid needle shield. The syringe is closed with a rubber coated (bromobutyl gray) plunger stopper.

In some embodiments, fitusiran is packaged in a prefilled pen containing 20 mg of fitusiran (equivalent to 21.2 mg fitusiran sodium). In some embodiments, each single-use prefilled pen contains 20 mg of fitusiran in 0.5 mL solution at a concentration of 40 mg/mL. In some embodiments, each prefilled pen is designed to deliver 20 mg of fitusiran in 0.5 mL which also contains: dibasic sodium phosphate (0.467 mg), monobasic sodium phosphate (0.105 mg), sodium chloride (3.836 mg), and Water for Injection, USP. Phosphoric acid (concentrated) and sodium hydroxide may be added to adjust to pH 7.0 for the prefilled pen. Fitusiran may be supplied as a sterile, clear solution for subcutaneous injection in the 20 mg PFP. In some embodiments, the PFP contains a single dose of fitusiran and comprises a PFS assembled with an automated injection system (pen). The PFS is a 1 mL colorless type 1 glass syringe with a 29 gauge, (½ inch) stainless steel staked needle covered with a rigid needle shield. The syringe is closed with a rubber coated (bromobutyl gray) plunger stopper.

In some embodiments, fitusiran is packaged in a vial containing 20 mg of fitusiran (equivalent to 21.2 mg fitusiran sodium). In these embodiments, each single-use vial contains 20 mg of fitusiran in 0.2 mL solution at a concentration of 100 mg/mL. In some embodiments, each vial contains 20 mg fitusiran in 0.2 mL which also contains: sodium chloride (0.982 mg), dibasic sodium phosphate (0.234 mg), monobasic sodium phosphate (0.018 mg), phosphoric acid (q.s. pH 7), sodium hydroxide (q.s. pH 7.0), and water for injection (q.s. 0.2 mL). Fitusiran may be supplied as a sterile, clear solution for subcutaneous injection in the vial. The vial comprises a single dose of fitusiran in a 2 mL colorless Type-1 glass vial with a bromobutyl rubber stopper and an aluminum polypropylene colored crimping cap. In certain embodiments, the composition of fitusiran solution for subcutaneous injection is shown in Table 1B below. For example, a 10 mg dose of fitusiran may be obtained from a 40 mg/mL fitusiran drug formulation or a 100 mg/mL fitusiran drug formulation, a 20 mg dose of fitusiran may be obtained from a 40 mg/mL fitusiran drug formulation, and a 50 mg dose may be obtained from a 100 mg/mL fitusiran drug formulation.

TABLE 1B
Fitusiran Formulations

| | | 20 mg | 50 mg |
|---|---|---|---|
| | Pediatric | Adult bPFS | Adult bPFS |
| | Formulation | Formulation | Formulation |
| Component | (mg/mL) | (mg/mL) | (mg/mL) |
| Fitusiran | 12.5 [13.25] | 40 [42.4] | 100 [106] |
| [Na salt] | | | |
| NaH₂PO₄*H₂O | 0.388 | 0.2095 | 0.0885 |
| Na₂HPO₄*7H₂O | 0.586 | 0.933 | 1.169 |
| NaCl (low | 8.7 | 7.672 | 4.909 |
| endotoxin) | | | |
| 0.1N NaOH | q.s. | q.s. | q.s. |
| 0.1M H₃PO₄ | q.s. | q.s. | q.s. |
| Purified water | Ad 1 mL | Ad 1 mL | Ad 1 mL |
| Compounded Bulk | Target 7.0 (6.8-7.2) | | |
| IPC pH | | | |
| Nitrogen | Processing Aid | | |
| Density (20° C.) | 1.0109 | 1.02364 | 1.05099 (Fixed) |
| Osmolality | Target = 300 | Target = 300 | Target = 300 |
| | mOsm/kg | mOsm/kg | mOsm/kg |
| Viscosity (mPa*s @ | 1.1 | 1.4 | 2.2 |
| 20° C.) | | | |

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH).

### II. Therapeutic Use of Fitusiran

Fitusiran is an antithrombin-directed small interfering ribonucleic acid and can suppress liver production of antithrombin (AT). In its role as an anti-coagulant, AT regulates hemostasis by directly targeting thrombin production or by inactivating uncomplexed FXa, which in turn reduces thrombin production (Quinsey et al., Int J Biochem Cell Biol. (2004) 36(3):386-9). Fitusiran may be used to treat those who have impaired hemostasis. For example, fitusiran can be used to treat patients with hemophilia A or B with or without inhibitors for routine prophylaxis to prevent or reduce the frequency of bleeding episodes. In particular embodiments, fitusiran is used to treat adult and adolescent patients (at least twelve years of age) with hemophilia A (congenital factor VIII deficiency) or hemophilia B (congenital factor IX deficiency) with or without inhibitors. The present methods include administering to the hemophilia patient (e.g., a hemophilia A or B patient) in need thereof a therapeutically effective amount of fitusiran. “Therapeutically effective amount” refers to the amount of fitusiran that helps the patient to achieve a desired clinical endpoint.

In some embodiments, fitusiran may be used for routine prophylaxis to prevent or reduce the frequency of bleeding episodes in adult and adolescent patients aged 12 years and older with hemophilia A or B with or without factor VIII or IX inhibitors. In certain embodiments, a fitusiran-containing pharmaceutical composition is administered by subcutaneous injection at a dose strength of, for example, about 5 to about 60 mg (e.g., about 5 to about 60 mg, about 10 to about 60 mg, about 15 to about 60 mg, about 20 to about 60 mg, about 25 to about 60 mg, about 30 to about 60 mg, about 35 to about 60 mg, about 40 to about 60 mg, about 45 to about 60 mg, about 50 to about 60 mg, about 5 to about 50 mg, about 5 to about 45 mg, about 5 to about 40 mg, about 5 to about 35 mg, about 5 to about 30 mg, about 5 to about 25 mg, about 5 to about 20 mg, about 5 to about 15 mg, or about 5 to about 10 mg) per dose. In particular embodiments, fitusiran is administered subcutaneously at about 10, about 20, or about 50 mg (weight of active moiety) per dose in a PBS solution as described above. In some embodiments, fitusiran is for subcutaneous use only.

In some embodiments, the dose of fitusiran may be adjusted for patients receiving fitusiran for routine prophylaxis in response to measured AT activity levels. In some embodiments, adjusting the dose of fitusiran reduces the risk of hepatotoxicity in a patient receiving fitusiran. In some embodiments, adjusting the dose of fitusiran reduces the risk of gallbladder disease or acute and recurrent gallbladder disease in a patient receiving fitusiran. In some embodiments, adjusting the dose of fitusiran reduces the thrombotic risk in a patient receiving fitusiran. In some embodiments, adjusting the dose of fitusiran prevents or reduces annualized bleeding rate in a patient receiving fitusiran. In some embodiments, the adjusted dose amounts of fitusiran are determined by a prescribing physician in response to one or more measurements of an AT level in the patient.

A plurality of fitusiran doses may be administered to a patient at an interval of about one, about two, about three, about four, about five, about six, about seven, or about eight weeks, or of about one, about two, or about three months. In particular embodiments, a fixed dose of fitusiran (e.g., about 50 mg subcutaneous injection, about 20 mg subcutaneous injection) is administered to a hemophilia patient (e.g., a hemophilia A or hemophilia B patient who is twelve years or older and who has or has not developed inhibitors) about once every four weeks or about once a month or about once every eight weeks or about once every other month. In some embodiments, fitusiran is administered at a frequency of QM, which can include administration once every month, about once every month, once every four weeks, or about once every four weeks. In some embodiments, fitusiran is administered at a frequency of Q2M, which can include administration once every two months, about once every two months, once every eight weeks, or about once every eight weeks. In some embodiments, fitusiran is administered at a frequency of Q4W, which can include administration once every four weeks, about once every four weeks, once every month, or about once every month. In some embodiments, fitusiran is administered at a frequency of Q8W, which can include administration once every eight weeks, about once every eight weeks, once every two months, or about once every two months.

In some embodiments, the present methods include subcutaneous administration of fitusiran at a dose of about 50 mg about once every two months, which may also be abbreviated as Q2M, i.e., 50 mg Q2M. In some embodiments, the present methods include subcutaneous administration of fitusiran at a dose of about 50 mg about once every eight weeks, which may also be abbreviated as Q8W, i.e., 50 mg Q8W. In some embodiments, the present methods include subcutaneous administration of fitusiran at a dose of about 50 mg about once every month, which may also be abbreviated as QM, i.e., 50 mg QM. In some embodiments, the present methods include subcutaneous administration of fitusiran at a dose of about 50 mg about once every four weeks, which may also be abbreviated as Q4W, i.e., 50 mg Q4W. In some embodiments, the present methods include subcutaneous administration of fitusiran at a dose of about 20 mg about once every two months, which may also be abbreviated as Q2M, i.e., 20 mg Q2M. In some embodiments, the present methods include subcutaneous administration of fitusiran at a dose of about 20 mg about once every eight weeks, which may also be abbreviated as Q8W, i.e., 20 mg Q8W. In some embodiments, the present methods include subcutaneous administration of fitusiran at a dose of about 20 mg about once every month, which may also be abbreviated as QM, i.e., 20 mg QM. In some embodiments, the present methods include subcutaneous administration of fitusiran at a dose of about 20 mg about once every four weeks, which may also be abbreviated as Q4W, i.e., 20 mg Q4W. In some embodiments, the present methods include subcutaneous administration of fitusiran at a dose of about 10 mg about once every two months, which may also be abbreviated as Q2M, i.e., 10 mg Q2M. In some embodiments, the present methods include subcutaneous administration of fitusiran at a dose of about 10 mg about once every eight weeks, which may also be abbreviated as Q8W, i.e., 10 mg Q8W. In some embodiments, the present methods include subcutaneous administration of fitusiran at a dose of about 10 mg about once every month, which may also be abbreviated as QM, i.e., 10 mg QM. In some embodiments, the present methods include subcutaneous administration of fitusiran at a dose of about 10 mg about once every four weeks, which may also be abbreviated as Q4W, i.e., 10 mg Q4W.

In some embodiments, the present methods involve measurement of AT levels in a patient. In some embodiments, the present methods involve obtaining a measurement of AT level in the human patient at about four weeks or one month, about eight weeks or two months, about every four months, about every six months, or about every twelve months following administration of the starting dose or following administration of a modified dose as compared to the prior dose. In some embodiments, the present methods involve obtaining a measurement of AT level in the human patient at about four weeks or one month, about twelve weeks or three months, about 20 weeks or five months, or about 24 weeks or six months following administration of the starting dose or following administration of a modified dose as compared to the prior dose.

AT measurements can be performed by well-established methods, including both kinetic and/or chromogenic assays. One commonly used method is the INNOVANCE™ Antithrombin assay (Siemens Healthineers, Malvern, PA; U.S. FDA 510(k) #K081769). INNOVANCE™ is a chromogenic assay that quantifies functionally active AT in human citrated plasma based on the inhibition of an excess of factor Xa by AT. The assay may be performed by using an automated coagulation instrument (e.g., Siemens BCS® XP, Sysmex® CA-600 and CS Systems, or Atellica® COAG 360 System), and may be calibrated using Siemens standard human plasma (SHP) with a defined value of AT activity calibrated against World Health Organization (WHO) reference plasma. An equivalent assay is the Dade Behring Berichrom™ Antithrombin III assay (Dade Behring Marburg GmbH, Marburg, Germany; U.S. FDA 510(k) #K933125). Each measurement may be controlled by two independent controls (low and normal value) also calibrated against the WHO standard. In some embodiments, one or more measurements of an AT level in the patient are obtained or have been obtained by a lab or technician, using any well-established method for obtaining AT measurements (e.g., the INNOVANCE™ Antithrombin assay or the Dade Behring Berichrom® Antithrombin III assay as described herein). The AT activity (%) in a plasma sample is calculated against the WHO reference plasma. 100% AT level is defined as 1 unit of antithrombin activity in 1 mL of reference plasma sample. The limit of detection of the INNOVANCE™ assay is 6.0% based on the assay's U.S. FDA 510(k) decision summary. AT levels range from about 80% to about 120% in the general population.

AT activity levels may be measured periodically during the course of treatment with fitusiran. In some embodiments, if one measurement of AT level is <15%, the present methods further comprise obtaining a second measurement of AT level. AT activity levels may also be measured specifically after a dose reduction or escalation. In some embodiments, the present methods further comprise obtaining a measurement of AT level at four weeks or one month, twelve weeks or three months, 20 weeks or five months, and 24 weeks or six months after a dose reduction in fitusiran. In some embodiments, the present methods further comprise obtaining a measurement of AT level at four weeks or one month, twelve weeks or three months, 20 weeks or five months, and 24 weeks or six months after a dose escalation in fitusiran.

The dose of fitusiran may be adjusted depending on specifically the second measurement of AT level. In some embodiments, the present methods further comprise subcutaneously administering to the human patient fitusiran at a lower dose amount 3 months after the prior dose if the second measurement of AT level is <15%. In some embodiments, the present methods further comprise discontinuing or pausing fitusiran treatment if the second measurement of AT level is <15%. The dose of fitusiran may also be adjusted depending on the length of time that AT activity is at a specific level. In some embodiments, the present methods further comprise administering to the human patient fitusiran at a higher dose amount if the AT level is >35% after six months. As an alternative measurement, the present methods may further comprise administering to the human patient fitusiran at a higher dose amount if the human patient has not achieved satisfactory bleed control.

In some embodiments, AT activity may be measured using a validated or approved AT activity assay or device. In some embodiments, AT activity is measured using an FDA-cleared test. In some embodiments, AT activity is measured using a validated test. In some embodiments, AT activity is measured using a companion diagnostic. For example, to monitor AT activity, a specific FDA-cleared human factor Xa based chromogenic activity assay is used. Bovine factor IIa based chromogenic assays, if used, may be validated at low AT activity (<15%). Bovine factor Xa based chromogenic assay may be avoided if the AT levels are below 35%. If an FDA-cleared human factor Xa based chromogenic activity assay is not locally available, a reference laboratory may be utilized.

In some embodiments, the starting dose of fitusiran is 50 mg once every two months (Q2M) for patients 12 years of age and older. In some embodiments, the starting dose of fitusiran is 50 mg once every two months (Q2M). In some embodiments, the starting dose of fitusiran is 20 mg Q2M. In some embodiments, the starting dose of fitusiran is 10 mg Q2M. In some embodiments, the dose is adjusted if needed, to maintain antithrombin (AT) activity between 15-35%. In some embodiments, the dose is adjusted depending on whether AT activity levels are <15%, between 15-35%, or >35% after 6 months. In some embodiments, the modified doses include 50 mg once every month (QM), 20 mg Q2M, 20 mg QM, 10 mg Q2M, or 10 mg QM. In some embodiments, fitusiran is discontinued depending on AT activity level.

It has been observed that the risk of serious thrombotic events among patients receiving fitusiran may increase with AT levels <15%. To mitigate the risk of serious thrombotic events while maintaining a favorable benefit-risk balance for patients on fitusiran, a patient, for example an adult patient (>18 years of age) or an adolescent patient (12 to 17 years of age, inclusive), may start on a fitusiran therapy by subcutaneous injection of 50 mg fitusiran every two months (or every eight weeks). The patient's AT level may be monitored periodically (e.g., every one, two, three, four, five, six, seven, or eight weeks, or every one, two, three, four, five, or six months). If the patient has two AT measures of <15%, a dose reduction in fitusiran treatment is required. A dose reduction can include a lower dose amount, a lower dose frequency, a pause in fitusiran treatment, or a discontinuation of fitusiran treatment. In some embodiments, upon the first AT level <15%, the patient has another AT activity level sample drawn prior to the next dose. If this second result is <15%, this will be considered the second AT<15%. Patients receiving fitusiran at a dose of 50 mg Q2M with more than 1 (e.g., 2) AT activity levels <15% require a dose reduction in fitusiran that should be initiated 3 months after the prior dose.

However, if the 50 mg/Q2M (or Q8W) patient has an AT measure of >35% after 6 months, the patient will escalate the dosing regimen. A dose escalation in fitusiran can include a higher dose amount or a higher dose frequency. Depending on the prior dose amount, the patient may receive fitusiran at 50 mg every month (or every four weeks), 20 mg every month (or every four weeks), or 10 mg every month (or every four weeks).

Patients who have more than one measurement (e.g., two) of AT activity level <15% when receiving fitusiran at a dose of 50 mg Q2M may receive fitusiran at a dose of 20 mg Q2M. Patients receiving fitusiran at a dose of 20 mg Q2M with more than one measurement (e.g., two) of AT activity level <15% may receive fitusiran at a dose of 10 mg Q2M. Patients receiving fitusiran at a dose of 10 mg Q2M with more than one measurement (e.g., two) of AT activity level <15% may discontinue fitusiran treatment. If a patient receiving fitusiran at a dose of 50 mg Q2M (or Q8W) has an AT measurement of >35% after 6 months, the patient may receive fitusiran at 50 mg QM (or Q4W). If a patient receiving fitusiran at a dose of 20 mg Q2M (or Q8W) has an AT measurement of >35% after 6 months, the patient may receive fitusiran at 20 mg QM (or Q4W). If a patient receiving fitusiran at a dose of 10 mg Q2M (or Q8W) has an AT measurement of >35% after 6 months, the patient may receive fitusiran at 10 mg QM (or Q4W).

In the above dose finding regimens, AT measurements for dosing determination are those taken during steady state (SS) of AT activity, i.e., once the patient's AT levels have been stabilized (at low AT activity range) after fitusiran treatment. The SS is typically reached after two or three doses of fitusiran. AT measurements for dosing determination are taken at an appropriate interval (e.g., every four weeks or every eight weeks).

In the above dose finding regimens, the starting dose of 50 mg fitusiran Q2M is included as an illustrative example. For example, a starting dose of fitusiran may be 50 mg Q2M, 20 mg Q2M, 20 mg QM, 10 mg Q2M, or 10 mg QM. Dose escalation and de-escalation can then be carried out accordingly from each starting dose. For example, a starting dose of 50 mg Q2M can be escalated to 50 mg QM or de-escalated to 20 mg Q2M. A starting dose of 20 mg Q2M fitusiran can be escalated to 20 mg QM, 50 mg Q2M, or 50 mg QM, optionally sequentially in that order, or de-escalated to 10 mg Q2M. A starting dose of 10 mg Q2M fitusiran can be escalated to 10 mg QM, or de-escalated to paused or discontinued treatment.

An AT level of 10-35% (e.g., 10-25%, 15-35%, or 15-25%) is targeted to mitigate the risk of vascular thrombotic events while maintaining a favorable benefit-risk balance for patients on fitusiran. Thus, so long as the patient reaches this targeted AT level, there is no need for the patient to receive a higher fitusiran dosage or more frequent dosing. That is, the patient can remain on the current treatment regimen (i.e., maintenance regimen). For example, once the desired AT level is reached, the patient may be treated with a subcutaneous dose of fitusiran (e.g., 40-90 mg per dose) at an interval of, e.g., every one, two, three, four, five, six, seven, or eight weeks, or every one, two, three, four, five, or six months. In some embodiments, if the patient has two AT measurements of no greater than 35% while receiving 50 mg Q2M, the patient will maintain this dosing regimen, with no need to further escalate the dosage or dosing frequency. As another example, if the patient has two AT measurements of no greater than 35% while receiving 50 mg QM, the patient will remain on this dosing regimen, with no need to further escalate the dosage or dosing frequency. However, fitusiran treatment should be de-escalated or discontinued if a patient has more than 1 (e.g., 2) AT measurements <15% as a risk mitigation measure for serious thrombotic events. The patient may resume treatment with a lower dose of fitusiran, optionally after their AT levels have returned to above 15%, e.g., >22%.

In some embodiments, if AT activity is consistently <15% and a dose reduction is required, the lower dose should be initiated three months after the prior dose. In some embodiments, AT activity should be measured prior to initiation of fitusiran treatment and then every other month prior to the next dose for six months. After a dose modification, AT measurements may be restarted.
If AT activity is consistently <15% and a dose reduction is required, the lower dose may be initiated three months after the prior dose.
If AT activity is >35% after six months, or if the patient has not achieved satisfactory bleed control, dose modification may be considered.
After the initial AT monitoring period, AT activity may be measured annually.

Recommended dose modification based on AT activity levels is shown in Table 2 below.

TABLE 2
Dose Modification Based on Antithrombin Activity Levels

| Last dosage | | |
|---|---|---|
| administered | Antithrombin Activity Level | Dose Modification |
| 50 mg every | Less than 15% | 20 mg every 2 months |
| 2 months | 15% to 35% | Continue current dosage |
| | Greater than 35% after 6 months | 50 mg every month* |
| 20 mg every | Less than 15% | 10 mg every 2 months |
| 2 months | 15% to 35% | Continue current dosage |
| | Greater than 35% after 6 months | 20 mg every month* |
| 10 mg every | Less than 15% | Discontinue fitusiran |
| 2 months | 15% to 35% | Continue current dosage |
| | Greater than 35% after 6 months | 10 mg every month* |

*For patients taking 50 mg QM, 20 mg QM, or 10 mg QM with AT levels >35%, continuation of treatment is subject to clinical judgement based on adequate bleed control.
QM = once every month, Q2M = once every two months.

In some embodiments of maintenance regimens, a patient with hemophilia A or B with or without inhibitors is treated with a subcutaneous dose of fitusiran at 50 mg per dose every two months (or every eight weeks). In other embodiments, a patient with hemophilia A or B with or without inhibitors is treated with a subcutaneous dose of fitusiran at 50 mg every month (or every four weeks). In yet other embodiments, a patient with hemophilia A or B with or without inhibitors is treated with a subcutaneous dose of fitusiran at 20 mg every two months (or every eight weeks). In yet other embodiments, a patient with hemophilia A or B with or without inhibitors is treated with a subcutaneous dose of fitusiran at 20 mg every month (or every four weeks). In yet other embodiments, a patient with hemophilia A or B with or without inhibitors is treated with a subcutaneous dose of fitusiran at 10 mg every two months (or every eight weeks). In yet other embodiments, a patient with hemophilia A or B with or without inhibitors is treated with a subcutaneous dose of fitusiran at 10 mg every month (or every four weeks).

In some embodiments, patients may receive periodic (e.g., monthly or every four weeks) AT monitoring for 12 months following a change in fitusiran dosing regimen.

In some embodiments, once a patient stays on a maintenance regimen (e.g., 10 mg QM or Q4W, 10 mg Q2M or Q8W, 20 mg Q2M or Q8W, 20 mg QM or Q4W, 50 mg Q2M or Q8W, 50 mg QM or Q4W), the patient may receive less frequent AT monitoring. For example, his AT level may be monitored every month, every two months, every three months, every four months, semi-annually, annually, or every two years.

In some embodiments, AT monitoring is initiated prior to the first dose of fitusiran treatment as shown in Table 2 above, and AT activity is measured using an FDA-specific validated AT activity assay. In some embodiments, fitusiran is administered at a starting dose and this dose is adjusted to any one of 50 mg QM, 20 mg Q2M, 20 mg QM, 10 mg Q2M, or 10 mg QM. In some embodiments, adjusting the dose of fitusiran means discontinuing or pausing treatment with fitusiran.

In some embodiments, the starting dose of fitusiran is 50 mg Q2M and this dose is adjusted, if needed, to maintain antithrombin (AT) activity between 15-35% (Table 2 above); the modified doses include 20 mg Q2M if AT activity is <15% and 50 mg QM if AT activity is >35% after 6 months. In some embodiments, the starting dose of fitusiran is 20 mg Q2M and this dose is adjusted, if needed, to maintain AT activity between 15-35%; the modified doses include 10 mg Q2M if AT activity is <15% and 20 mg QM if AT activity is >35% after 6 months. In some embodiments, the starting dose of fitusiran is 10 mg Q2M and this dose is adjusted, if needed, to maintain AT activity between 15-35%; the modified doses include discontinuing fitusiran treatment if AT activity is <15% and 10 mg QM if AT activity is >35% after 6 months.

In one aspect, provided herein is a method of reducing a risk of hepatotoxicity in a human patient with hemophilia A or B with or without factor VIII or factor IX inhibitors who receives fitusiran for routine prophylaxis to prevent or reduce the frequency of bleeding episodes. In some embodiments, the method comprises (1) (a) administering to the human patient fitusiran at a starting dose of 50 mg about once every 2 months; and (b) performing one of the following steps: (i) if an antithrombin (AT) level in the human patient is 15-35%, subcutaneously administering to the human patient fitusiran at a dose of 50 mg about once every 2 months, (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (1)(a), after step (1)(a), and after any fitusiran dose modification; (2) (a) administering to the human patient fitusiran at a dose of 20 mg about once every 2 months; and (b) performing one of the following steps: (i) if an AT level in the human patient is 15-35%, subcutaneously administering to the human patient fitusiran at a dose of 20 mg about once every 2 months, (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (2)(a), after step (2)(a), and after any fitusiran dose modification; or (3) (a) after administering to the human patient fitusiran at a dose of 10 mg about once every 2 months; and (b) performing one of the following steps: (i) if an AT level in the human patient is 15-35%, subcutaneously administering to the human patient fitusiran at a dose of 10 mg about once every 2 months, (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (3)(a), after step (3)(a), and after any fitusiran dose modification. In some embodiments, the method comprises (a) subcutaneously administering to the human patient in need thereof fitusiran at a dose of 50 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (a), after step (a), and after any fitusiran dose modification. In some embodiments, the method comprises (a) subcutaneously administering to the human patient in need thereof fitusiran at a dose of 20 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (a), after step (a), and after any fitusiran dose modification. In some embodiments, the method comprises (a) subcutaneously administering to the human patient in need thereof fitusiran at a dose of 10 mg about once every 2 months; (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (a), after step (a), and after any fitusiran dose modification. In some embodiments, steps (a) and (b) are performed by the patient, a caregiver, or a healthcare professional. In some embodiments, the one or more measurements of AT level in the patient are or have been obtained by a lab or technician. In some embodiments of this aspect, a prescribing physician determines the dosage amounts of fitusiran in the patient in response to the obtained one or more measurements of AT level.

In another aspect, provided herein is a method of reducing a risk of gallbladder disease in a human patient with hemophilia A or B with or without factor VIII or factor IX inhibitors who receives fitusiran for routine prophylaxis to prevent or reduce the frequency of bleeding episodes. In some embodiments, the method comprises: (1) (a) administering to the human patient fitusiran at a starting dose of 50 mg about once every 2 months; and (b) performing one of the following steps: (i) if an antithrombin (AT) level in the human patient is 15-35%, subcutaneously administering to the human patient fitusiran at a dose of 50 mg about once every 2 months, (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (1)(a), after step (1)(a), and after any fitusiran dose modification; (2) (a) administering to the human patient fitusiran at a dose of 20 mg about once every 2 months; and (b) performing one of the following steps: (i) if an AT level in the human patient is 15-35%, subcutaneously administering to the human patient fitusiran at a dose of 20 mg about once every 2 months, (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (2)(a), after step (2)(a), and after any fitusiran dose modification; or (3) (a) after administering to the human patient fitusiran at a dose of 10 mg about once every 2 months; and (b) performing one of the following steps: (i) if an AT level in the human patient is 15-35%, subcutaneously administering to the human patient fitusiran at a dose of 10 mg about once every 2 months, (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (3)(a), after step (3)(a), and after any fitusiran dose modification. In some embodiments, the method comprises: (a) subcutaneously administering to the human patient in need thereof fitusiran at a dose of 50 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (a), after step (a), and after any fitusiran dose modification. In some embodiments, the method comprises: (a) subcutaneously administering to the human patient in need thereof fitusiran at a dose of 20 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (a), after step (a), and after any fitusiran dose modification. In some embodiments, the method comprises: (a) subcutaneously administering to the human patient in need thereof fitusiran at a dose of 10 mg about once every 2 months; (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (a), after step (a), and after any fitusiran dose modification. In some embodiments, steps (a) and (b) are performed by the patient, a caregiver, or a healthcare professional. In some embodiments, the one or more measurements of AT level in the patient are or have been obtained by a lab or technician. In some embodiments of this aspect, a prescribing physician determines the dosage amounts of fitusiran in the patient in response to the obtained one or more measurements of AT level.

In another aspect, provided herein is a method of reducing thrombotic risk in a human patient with hemophilia A or B with or without factor VIII or factor IX inhibitors who receives fitusiran for routine prophylaxis to prevent or reduce the frequency of bleeding episodes. In some embodiments, the method comprises: (1) (a) administering to the human patient fitusiran at a starting dose of 50 mg about once every 2 months; and (b) performing one of the following steps: (i) if an antithrombin (AT) level in the human patient is 15-35%, subcutaneously administering to the human patient fitusiran at a dose of 50 mg about once every 2 months, (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (1)(a), after step (1)(a), and after any fitusiran dose modification; (2) (a) administering to the human patient fitusiran at a dose of 20 mg about once every 2 months; and (b) performing one of the following steps: (i) if an AT level in the human patient is 15-35%, subcutaneously administering to the human patient fitusiran at a dose of 20 mg about once every 2 months, (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (2)(a), after step (2)(a), and after any fitusiran dose modification; or (3) (a) after administering to the human patient fitusiran at a dose of 10 mg about once every 2 months; and (b) performing one of the following steps: (i) if an AT level in the human patient is 15-35%, subcutaneously administering to the human patient fitusiran at a dose of 10 mg about once every 2 months, (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (3)(a), after step (3)(a), and after any fitusiran dose modification. In some embodiments, the method comprises: (a) subcutaneously administering to the human patient in need thereof fitusiran at a dose of 50 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (a), after step (a), and after any fitusiran dose modification. In some embodiments, the method comprises: (a) subcutaneously administering to the human patient in need thereof fitusiran at a dose of 20 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (a), after step (a), and after any fitusiran dose modification. In some embodiments, the method comprises: (a) subcutaneously administering to the human patient in need thereof fitusiran at a dose of 10 mg about once every 2 months; (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (a), after step (a), and after any fitusiran dose modification. In some embodiments, steps (a) and (b) are performed by the patient, a caregiver, or a healthcare professional. In some embodiments, the one or more measurements of AT level in the patient are or have been obtained by a lab or technician. In some embodiments of this aspect, a prescribing physician determines the dosage amounts of fitusiran in the patient in response to the obtained one or more measurements of AT level.

In another aspect, provided herein is a method of routine prophylaxis to prevent or reduce the frequency of bleeding episodes in a human patient with hemophilia A or B with or without factor VIII or factor IX inhibitors. In some embodiments, the method comprises: (1) (a) administering to the human patient fitusiran at a starting dose of 50 mg about once every 2 months; and (b) performing one of the following steps: (i) if an antithrombin (AT) level in the human patient is 15-35%, subcutaneously administering to the human patient fitusiran at a dose of 50 mg about once every 2 months, (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (1)(a), after step (1)(a), and after any fitusiran dose modification; (2) (a) administering to the human patient fitusiran at a dose of 20 mg about once every 2 months; and (b) performing one of the following steps: (i) if an AT level in the human patient is 15-35%, subcutaneously administering to the human patient fitusiran at a dose of 20 mg about once every 2 months, (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (2)(a), after step (2)(a), and after any fitusiran dose modification; or (3) (a) after administering to the human patient fitusiran at a dose of 10 mg about once every 2 months; and (b) performing one of the following steps: (i) if an AT level in the human patient is 15-35%, subcutaneously administering to the human patient fitusiran at a dose of 10 mg about once every 2 months, (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (3)(a), after step (3)(a), and after any fitusiran dose modification. In some embodiments, the method comprises: (a) subcutaneously administering to the human patient in need thereof fitusiran at a dose of 50 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (a), after step (a), and after any fitusiran dose modification. In some embodiments, the method comprises: (a) subcutaneously administering to the human patient in need thereof fitusiran at a dose of 20 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (a), after step (a), and after any fitusiran dose modification. In some embodiments, the method comprises: (a) subcutaneously administering to the human patient in need thereof fitusiran at a dose of 10 mg about once every 2 months; (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (a), after step (a), and after any fitusiran dose modification. In some embodiments, steps (a) and (b) are performed by the patient, a caregiver, or a healthcare professional. In some embodiments, the one or more measurements of AT level in the patient are or have been obtained by a lab or technician. In some embodiments of this aspect, a prescribing physician determines the dosage amounts of fitusiran in the patient in response to the obtained one or more measurements of AT level.

In another aspect, provided herein is a method of reducing annualized bleeding rate in a human patient with hemophilia A or B with or without factor VIII or factor IX inhibitors who receives fitusiran for routine prophylaxis to prevent or reduce the frequency of bleeding episodes. In some embodiments, the method comprises: (1) (a) administering to the human patient fitusiran at a starting dose of 50 mg about once every 2 months; and (b) performing one of the following steps: (i) if an antithrombin (AT) level in the human patient is 15-35%, subcutaneously administering to the human patient fitusiran at a dose of 50 mg about once every 2 months, (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (1)(a), after step (1)(a), and after any fitusiran dose modification; (2) (a) administering to the human patient fitusiran at a dose of 20 mg about once every 2 months; and (b) performing one of the following steps: (i) if an AT level in the human patient is 15-35%, subcutaneously administering to the human patient fitusiran at a dose of 20 mg about once every 2 months, (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (2)(a), after step (2)(a), and after any fitusiran dose modification; or (3) (a) after administering to the human patient fitusiran at a dose of 10 mg about once every 2 months; and (b) performing one of the following steps: (i) if an AT level in the human patient is 15-35%, subcutaneously administering to the human patient fitusiran at a dose of 10 mg about once every 2 months, (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (3)(a), after step (3)(a), and after any fitusiran dose modification. In some embodiments, the method comprises: (a) subcutaneously administering to the human patient in need thereof fitusiran at a dose of 50 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 50 mg about once every month, or (iii) if an AT level in the human patient is <15%, subcutaneously administering to the human patient fitusiran at a dose of 20 mg about once every 2 months, wherein one or more measurements of an AT level in the human patient are or have been obtained before step (a), after step (a), and after any fitusiran dose modification. In some embodiments, the method comprises: (a) subcutaneously administering to the human patient in need thereof fitusiran at a dose of 20 mg about once every 2 months; and (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 20 mg about once every month, or (iii) if an AT level in the human patient is <15%, subcutaneously administering to the human patient fitusiran at a dose of 10 mg about once every 2 months, wherein one or more measurements of an AT level in the human patient are or have been obtained before step (a), after step (a), and after any fitusiran dose modification. In some embodiments, the method comprises: (a) subcutaneously administering to the human patient in need thereof fitusiran at a dose of 10 mg about once every 2 months; (b) performing one of the following steps: (i) if an antithrombin (AT) level in the human patient is 15-35%, repeating step (a), (ii) if an AT level in the human patient is >35% after 6 months, subcutaneously administering to the human patient fitusiran at a dose of 10 mg about once every month, or (iii) if an AT level in the human patient is <15%, discontinuing or pausing fitusiran treatment, wherein one or more measurements of an AT level in the human patient are or have been obtained before step (a), after step (a), and after any fitusiran dose modification. In some embodiments, steps (a) and (b) are performed by the patient, a caregiver, or a healthcare professional. In some embodiments, the one or more measurements of AT level in the patient are or have been obtained by a lab or technician. In some embodiments of this aspect, a prescribing physician determines the dosage amounts of fitusiran in the patient in response to the obtained one or more measurements of AT level.

After cessation of fitusiran dosing, routine AT monitoring may not be needed unless the patient is bleeding and treatment with CFC/BPA is required. Based on data from the clinical studies, the majority of patients have an AT activity >60% by 6 months after the last dose, after which normal doses of CFC/BPA may be used.

In some embodiments, AT activity is measured at about 4 weeks or about 1 month, about 12 weeks or about 3 months, about 20 weeks or about 5 months, and about 24 weeks or about 6 months following a starting dose of fitusiran and after any dose modifications. In some embodiments, if AT activity is <15%, another sample measuring AT activity is obtained prior to the next dose to determine if a dose modification is needed. If a second AT activity test is <15%, a dose reduction is required. The lower dose should be initiated 3 months after the prior dose. AT measurements should be restarted after a dose reduction. In some embodiments, if AT activity is >35% after 6 months, or if the patient has not achieved satisfactory bleed control, dose escalation should be considered. AT measurements should be restarted after a dose escalation.

In some embodiments, if a dose of fitusiran is missed, fitusiran is administered as soon as possible; thereafter, the dosing schedule is resumed either once every month or once every two months, as applicable, from the last dose.

In some embodiments, the patient receiving fitusiran therapy has been on routine prophylactic use of replacement factor or bypassing agent (BPA). Patients undergoing prophylactic treatment with replacement factor or BPA are typically treated as described in Table 3, wherein factor and bypassing agents are administered intravenously.

TABLE 3
Factor or bypassing agent prophylaxis requirements

| Type of Factor or BPA | Frequency of |
|---|---|
| Replacement | Administration |
| Standard half-life FVIII | Twice weekly |
| Extended half-life FVIII | Once weekly |
| Standard half-life FIX | Once weekly |
| Extended half-life FIX | Once biweekly |
| aPCC | Twice weekly |
| rFVIIa | Every other day |

After fitusiran therapy is initiated, these patients may continue their prior replacement factor or bypassing agent (BPA) prophylaxis for the first seven days of treatment, and after the first seven days of fitusiran treatment, the factor or BPA prophylaxis is discontinued. Subsequent to day seven of the fitusiran treatment period, factor concentrates or BPAs are administered only for bleeding episodes or if needed in advance of invasive medical procedures.

In some embodiments, fitusiran is administered subcutaneously into the thigh or abdomen. In some embodiments, a caregiver injects fitusiran in the outer area of the patient's upper arm. In some embodiments, fitusiran is not inject into a vein.

### III. Bleed Management

Patients receiving fitusiran prophylactic therapy herein may have breakthrough bleed episodes or may have surgery-related bleed episodes. The factor or BPA dose necessary to safely and effectively treat bleed episodes in patients receiving fitusiran during the fitusiran treatment period will be lower than standardly prescribed. Clinical experience suggests that pharmacodynamic (PD) drug interactions between fitusiran and concomitantly administered clotting factor concentrates (CFC) or BPA may occur when higher or more frequent doses of CFC or BPA than recommended in the breakthrough bleed management guidance (Table 4) are used.

In some embodiments, for patients using on-demand treatment with CFC or BPA after fitusiran initiation, the prior dosing regimen of CFC or BPA therapy may be used to treat breakthrough bleeding episodes only for the first seven days.

If breakthrough bleeding occurs after seven days of the first fitusiran dose, bleeds may be managed with a reduced dose and frequency of CFC/BPA to minimize the risk of thrombotic events. Initially, the weight-based dose of a CFC/BPA may be halved, and the dosing interval doubled compared to the routine dose (Table 4). If adequate hemostatic control is not achieved, higher doses may be used per clinical judgement. In some embodiments, antifibrinolytics with CFC or BPA are not used in combination.

TABLE 4
Bleed Management Guidance for Breakthrough Bleeding Occurring
Eight or More Days After the First Fitusiran Dose

| | | Factor IX | Factor IX | | |
|---|---|---|---|---|---|
| | Factor VIII | Standard Half-Life | Extended Half-Life | aPCC | rFVIIa |
| Recommended dose | 10 IU/kg | 20 IU/kg | 20 IU/kg | 30 U/kg | ≤45 μg/kg |
| Maximum dose | 20 IU/kg* | 30 IU/kg* | 30 IU/kg* | 50 U/kg* | 45 μg/kg |
| Repeat dosing | Should not repeat | Should not repeat | Should not repeat | Should not repeat | Should not repeat |
| | in <24 hours | in <24 hours | in <5-7 days | in <24 hours | in <2 hours |

*Use clinical judgment for situations requiring higher doses, more frequent administration, or multiple repeat doses.
Use standard of care for adjunctive management of bleeding episodes and cases of thrombosis.
aPCC = activated prothrombin complex concentrate; rFVIIa = activated recombinant Factor VII.

Under the above management guidelines, after the first seven days of the first fitusiran dose, when a patient experiences a symptom that may be consistent with bleeding episodes, the following steps are followed:
A single dose is administered according to the guidelines in Table 4.
The patient is instructed to re-evaluate symptoms in 24 hours for bleeds treated with FVIII, FIX or aPCC and in 2-3 hours for bleeds treated with rFVIIa.
   Administration of FIX Extended half-life is not to be more frequent than every 5-7 days.
Doses are not administered at less than 24-hour intervals (except rFVIIa as indicated in Table 4).
Doses do not exceed the protocol maximum recommended dose indicated in Table 4.

In some embodiments, the bleeding episode is a minor bleeding episode. In some embodiments, the bleeding episode is a moderate bleeding episode. In some embodiments, the bleeding episode is a severe bleeding episode.

In some embodiments, the bleed management guidelines maintain hemostasis in a patient undergoing a major surgery. As used herein, a “major surgery” includes, for example, opening into a major body cavity (e.g., abdomen, thorax, or skull), operation on a joint, removal of an organ, operative alteration of normal anatomy, crossing of a mesenchymal barrier (e.g., pleura, peritoneum, or dura), dental extraction of molar teeth or >3 non-molar teeth, or tooth implantation. In some embodiments, major orthopedic surgery is an operation on a joint or bone and associated soft tissue. In some embodiments, the peri-operative hemostatic control is assessed based on the ISTH 4-point response scale (excellent/good/moderate/poor). In some embodiments, the patient is not being treated with AT replacement therapy before surgery. In some embodiments, the patient is not being treated with AT replacement therapy during surgery. In some embodiments, the patient is not being treated with AT replacement therapy after surgery.

In some embodiments, reduced doses of replacement factor or bypassing agent are used as perioperative prophylaxis. In some embodiments, no replacement factor or bypassing agent is used as perioperative prophylaxis. In some embodiments, hemostatic control on the day of the surgery is rated excellent or good. In some embodiments, ATIII concentrate is used to reverse the pharmacodynamic effect of fitusiran in surgeries with an excellent/good hemostatic outcome.

### IV. Selection of Patients

The present treatments may be used to prophylactically treat patients with hemophilia A or hemophilia B with or without inhibitors. In some embodiments, the patient is a male aged >12 years with severe hemophilia A or B with or without inhibitors. A diagnosis of severe hemophilia is based on Factor VIII level of <1% (for hemophilia A) or a Factor IX level of <2% (for hemophilia B). In some embodiments, the patient is a male aged >18 years with moderate or severe hemophilia A or B with or without inhibitors. A diagnosis of moderate or severe hemophilia is based on FVIII or FIX level ≤5%.

In some embodiments, the patient has used replacement factors or bypassing agents prophylactically to manage bleeding episodes for at least six months prior to the start of treatment. In some embodiments, these patients have inhibitory antibodies to factor VIII or factor IX, and have experienced a minimum of two bleeding episodes requiring BPA treatment within the last six months prior to treatment. In some embodiments, these patients are without inhibitory antibodies to factor VIII or factor IX, and have experienced a minimum of one bleeding episode requiring factor treatment within the last twelve months prior to treatment.

In some embodiments, the patient has not used replacement factors or bypassing agents prophylactically to manage bleeding episodes for at least six months prior to the start of treatment. In some embodiments, these patients have experienced a minimum of six bleeding episodes requiring BPA (inhibitor participants) or factor (non-inhibitor participants) treatment within the last six months prior to treatment.

In some embodiments, the patient has inhibitory antibodies to Factor VIII or Factor IX (i.e., is a patient with inhibitors). A patient is defined as a patient with inhibitors if they meet at least one of the following Nijmegen-modified Bethesda assay results criteria:
a. inhibitor tier of >0.6 BU/mL prior to treatment, or
b. inhibitor titer of <0.6 BU/mL prior to treatment with medical record evidence of two consecutive titers >0.6 BU/mL, or
c. inhibitor titer of <0.6 BU/mL prior to treatment with medical record evidence of anamnestic response.

In some embodiments, the patient does not have inhibitory antibodies to Factor VIII or Factor IX (i.e., is a patient without inhibitors). A patient is defined as a patient without inhibitors if they meet at least one of the following Nijmegen-modified Bethesda assay results criteria:
a. Nijmegen-modified Bethesda assay inhibitor titer of <0.6 BU/mL prior to treatment,
b. no use of bypassing agents to treat bleeding episodes for at least the last six months prior to treatment, and
c. no history of immune tolerance induction therapy within the past three years prior to treatment.

In some embodiments, the patient does not have a known co-existing bleeding disorder other than hemophilia A or B, e.g., Von Willebrand's disease, additional factor deficiencies, or platelet disorders. The patient may not have a co-existing thrombophilic disorder, as determined by presence of any of the below:
a. FV Leiden mutation (homozygous or heterozygous),
b. protein S deficiency,
c. protein C deficiency, or
d. prothrombin mutation (G20210A; homozygous and heterozygous).

In some embodiments, the patient does not have a history of antiphospholipid antibody syndrome or arterial or venous thromboembolism, atrial fibrillation, significant valvular disease, myocardial infarction, angina, transient ischemic attack, or stroke. Patients who have experienced thrombosis associated with indwelling venous access may be treated with the present methods.

In some embodiments, the patient has not had a malignancy within two years, except for basal or squamous cell carcinoma of the skin that has been successfully treated.

In some embodiments, the patient is not currently participating in immune tolerance induction therapy (ITI).

In some embodiments, the patient has not used compounds, other than factor concentrates or BPAs for hemophilia treatment (including emicizumab (Hemlibra®)) within six months prior to treatment.

In some embodiments, the patient does not have an ALT and/or AST measurement >1.5×upper limit of normal reference range (ULN). In some embodiments, the patient does not have an ALT and/or AST measurement >5×ULN. The patient may not have an AT activity <60% prior to treatment. In some embodiments, the patient does not have a clinically significant liver disease, as indicated by (i) history of portal hypertension, esophageal varices, or hepatic encephalopathy; or (ii) presence of ascites by physical exam. In some embodiments, the patient does not have total bilirubin levels >1.5×ULN, or >2.0×ULN in participants with Gilbert's Syndrome. In some embodiments, the patient does not have uncontrolled hypertension defined as systolic blood pressure >160 mmHg and diastolic blood pressure >100 mmHg.

In some embodiments, fitusiran is not used in patients with underlying hepatic diseases or moderate to severe hepatic impairment.

In some embodiments, baseline liver function tests (LFTs), including AST and ALT, are obtained in all patients initiating fitusiran prophylaxis and then be monitored every two months for a period of at least six months. In some embodiments, if AST or ALT remain elevated above baseline, LFT monitoring is continued periodically thereafter.

In some embodiments, if new or worsening liver dysfunction is observed, appropriate diagnostic evaluations are performed. In some embodiments, medical management is initiated as appropriate and laboratory parameters are monitored until normalization to baseline. In some embodiments, if transaminase levels show evidence of progression, particularly if they rise to greater than five times ULN and are persistent, or if they are associated with symptoms, the present treatment methods are interrupted. In some embodiments, the benefits and risks of resuming fitusiran prophylaxis following resolution of transaminase elevations are considered.

In some embodiments, the patient is not Hepatitis C virus antibody positive. A patient who is Hepatitis C virus antibody positive may be treated with the present methods only if they have:
a. completed curative treatment at least 12 weeks prior to enrollment and attained sustained virologic response as documented by a negative HCV RNA prior to treatment, or they have spontaneously cleared infection as documented by negative HCV RNA prior to treatment, and
b. no evidence of cirrhosis according to FibroScan <12.5 kPa (where available), or FibroTest score <0.75 and APRI <2 (if FibroScan unavailable).

In some embodiments, the patient does not have acute hepatitis, i.e., hepatitis A or hepatitis E. In some embodiments, the patient does not have acute or chronic hepatitis B infection (IgM anti-HBc antibody positive or HBsAg positive).

In some embodiments, the patient does not have (i) a platelet count ≤100,000/μL, (ii) is not HIV positive with CD4 count <200 cells/μL, and/or (iii) does not have an estimated glomerular filtration rate ≤45 mL/min/1.73 m² (using the Modification of Diet in Renal Disease [MDRD] formula).

In some embodiments, the patient is not a pregnant woman. In some embodiments, fitusiran is used during pregnancy only if the potential benefit justifies the potential risks, including those to the fetus.

In some embodiments, the patient is not breastfeeding. In some embodiments, the developmental and health benefits of breastfeeding is considered along with the mother's clinical need for fitusiran and any potential adverse effects on the breastfed infant from fitusiran or from the underlying maternal condition.

In some embodiments, women of childbearing potential use non-hormonal contraception prior to starting treatment with and while receiving fitusiran.

In some embodiments, the patient is 65 years old.

In some embodiments, the patient has mild renal impairment (eGFR & 60 to <90 mL/min/1.73 m²). In some embodiments, the patient has moderate renal impairment (eGFR 30 mL/min/1.73 m² to <60 mL/min/1.73 m²).

In some embodiments, the patient does not have a corrected QT(QTc) interval 450 ms. The patient may not have an international normalized ratio (INR) of >1.5.

In some embodiments, the patient does not have (i) clinically significant liver disease, (ii) ALT >1.5×upper limit of normal reference range (ULN), (iii) AST >1.5×upper limit of normal reference range (ULN), (iv) hepatitis C, (v) hepatitis A, (vi) hepatitis E, (vii) hepatitis B, (viii) acute and recurrent gallbladder disease, (ix) symptomatic gallbladder disease, (x) established thrombophilic conditions, (xi) a prior history of thrombosis, (xii) an indwelling venous catheter, (xiii) persistent AT activity <15%, or (xiv) known hepatic impairment. In some embodiments, the known hepatic impairment is Child-Pugh Class A, B, or C.

In some embodiments, the patient meets one or more of the inclusion criteria and one or more of the exclusion criteria of the clinical studies that are described in Example 1 below.

### V. Treatment Outcomes and Evaluation

In some embodiments, the present treatment methods reduce the frequency of bleeding episodes in a patient. A bleeding episode is defined as any occurrence of hemorrhage that requires replacement factor or BPA infusion, e.g., hemarthrosis, muscle, or mucosal bleeding. The definition of bleeding episode types described below is based on consensus opinion of International Society on Thrombosis and Haemostasis (ISTH) (Blanchette et al., J Thromb Haemost. (2014) 12(11):1935-9).

The start time of a bleeding episode is defined as the time at which symptoms of a bleeding episode first develop. Bleeding or any symptoms of bleeding at the same location that occurs within 72 hours of the last injection used to treat a bleeding episode at that location is considered a part of the original bleeding event, and will count as one bleeding episode towards the ABR. Any bleeding symptoms that begin more than 72 hours from the last injection used to treat a bleeding episode at that location will constitute a new bleeding event.

A spontaneous bleeding episode refers to a bleeding event that occurs for no apparent or known reason, particularly into the joints, muscles, and soft tissues. A joint bleeding episode is characterized by an unusual sensation in the joint (“aura”) in combination with 1) increasing swelling or warmth over the skin over the joint, 2) increasing pain, or 3) progressive loss of range of motion or difficulty in using the limb as compared with baseline. A muscle bleed may be characterized by pain, swelling and loss of movement over the affected muscle group. A target joint is defined as a joint where three or more spontaneous bleeding episodes in a single joint within a consecutive six-month period has occurred; where there have been less than or equal to two bleeding episodes in the joint within a consecutive 12-month period the joint is no longer considered a target joint. A traumatic bleeding episode is one that is caused by a known injury or trauma. Bleeding episodes sustained during sports and recreation is counted as traumatic bleeding episodes.

In some embodiments, the present treatment methods result in reduction in the annualized bleed rate (ABR), and/or the annualized spontaneous bleeding rate (AsBR) in the treated population compared to a population treated prophylactically with compared to another prophylaxis treatment (e.g., replacement factor, BPA, or emicizumab).

According to the International Conference on Harmonisation (ICH) E2A guideline Definitions and Standards for Expedited Reporting, and 21 Code of Federal Regulations (CFR) 312.32, IND Safety Reporting, an adverse event (AE) is any untoward medical occurrence in a patient or clinical investigational patient administered a medicinal product and which does not necessarily have a causal relationship with this treatment. Since bleeding episodes are recorded as an efficacy assessment of fitusiran, these will not be treated as AEs unless they meet any of the severe adverse event (SAE) criteria listed below.

An SAE is any untoward medical occurrence that at any dose:
a. results in death,
b. is life-threatening (an event which places the patient at immediate risk of death from the event as it occurred. It does not include an event that had it occurred in a more severe form might have caused death),
c. requires in-patient hospitalization or prolongation of existing hospitalization,
d. results in persistent or significant disability or incapacity,
e. is a congenital anomaly or birth defect, or
f. is an important medical event that may not be immediately life-threatening or result in death or hospitalization but may jeopardize the patient and may require intervention to prevent one of the other outcomes listed in the definition above (e.g., events include allergic bronchospasm requiring intensive treatment in an emergency room or at home, blood dyscrasias, convulsions, or the development of drug dependency or abuse).

In some embodiments, the present treatment methods reduce the risk of hepatotoxicity in a human patient receiving fitusiran for prophylactic treatment of hemophilia A or B with or without factor VIII or factor IX inhibitors. These methods use lower dosage of fitusiran, or allow AT-based downward titration of fitusiran dosage to a level that is appropriate for individual patients, such that hepatotoxicity is reduced (e.g., fitusiran dosage at 50 mg Q2M, 20 mg QM, 20 Q2M, 10 mg QM, 10 mg Q2M). In some embodiments, the hepatotoxicity is ALT elevation >3× upper limit of normal (ULN), AST elevation >3× upper limit of normal (ULN), severe liver toxicity, liver failure, cholecystitis, cholelithiasis, or need for cholecystectomy. In some embodiments, the risk of hepatotoxicity is reduced as compared to the risk of hepatotoxicity in a patient treated with the original dose regimen (e.g., a regimen of 50 mg Q2M, 20 mg Q2M, 10 mg Q2M). In some embodiments, the exposure adjusted incidence rate (per 100 patient years) of cholecystitis/cholelithiasis is about 2.26 in patients treated with the AT-based dose regimen, compared to about 14.67 in patients treated with the original dose regimen. In some embodiments, the exposure adjusted incidence rate (per 100 patient years) of ALT/AST elevations >3×ULN is about 2.06 in patients treated with the AT-based dose regimen, compared to about 18.25 in patients treated with the original dose regimen. In some embodiments, ALT and AST elevations observed in patients treated with the AT-based dose regimen are transient.

In some embodiments, the method for reducing the risk of hepatotoxicity further comprises obtaining a liver function test in the human patient every month following initiation of fitusiran treatment. In some embodiments, the liver function test is an aspartate transaminase test. In some embodiments, the liver function test is an alanine aminotransferase test. In some embodiments, the liver function test is a measurement of total bilirubin. In some embodiments, the method further comprises obtaining a liver function test every month for the first six months following initiation of fitusiran treatment. In some embodiments, the liver function test is obtained in the human patient (i) prior to administering the starting dose of fitusiran to determine a baseline level of liver function; and (ii) about every month following initiation of fitusiran treatment. In some embodiments, the results of the liver function test guide subsequent treatment with fitusiran. In some embodiments, fitusiran treatment is resumed if the aspartate transaminase or alanine aminotransferase test shows aspartate transaminase or alanine aminotransferase levels have returned to baseline level and if the total bilirubin level has returned to baseline.

In some embodiments, the present treatment methods reduce the risk of gallbladder disease in a human patient receiving fitusiran for prophylactic treatment of hemophilia A or B with or without factor VIII or factor IX inhibitors. These methods use lower dosage of fitusiran, or allow AT-based downward titration of fitusiran dosage to a level that is appropriate for individual patients, such that the risk of gallbladder disease is reduced (e.g., fitusiran dosage at 50 mg Q2M, 20 mg QM, 20 Q2M, 10 mg QM, 10 mg Q2M). In some embodiments, the risk of gallbladder disease comprises a risk of cholecystitis, cholelithiasis, or a need for cholecystectomy. In some embodiments, reducing the risk of gallbladder disease comprises reducing the occurrence in the human patient of cholecystitis, cholelithiasis, or a need for cholecystectomy. In some embodiments, the gallbladder disease is acute and recurrent gallbladder disease. In some embodiments, the method further comprises discontinuing or pausing fitusiran treatment if the human patient is diagnosed with gallbladder disease.

In some embodiments, the present treatment methods reduce the thrombotic risk in a human patient receiving fitusiran for prophylactic treatment of hemophilia A or B with or without factor VIII or factor IX inhibitors. These methods use lower dosage of fitusiran, or allow AT-based downward titration of fitusiran dosage to a level that is appropriate for individual patients, such that the thrombotic risk is reduced (e.g., fitusiran dosage at 50 mg Q2M, 20 mg QM, 20 Q2M, 10 mg QM, 10 mg Q2M). In some embodiments, the thrombotic risk comprises a risk of a serious thrombotic event. In some embodiments, the serious thrombotic event comprises cerebrovascular accident, cerebral artery embolism, spinal vascular disorder, atrial thrombosis, cerebral venous sinus thrombosis, thrombosis, thrombosis on papillae of an eye, deep venous thrombosis of an arm, subclavian vein thrombosis, superficial venous thrombosis of an arm, cerebral infarction, embolic stroke, or postoperative venous thrombosis. In some embodiments, reducing thrombotic risk comprises reducing the occurrence in the human patient of cerebrovascular accident, cerebral artery embolism, spinal vascular disorder, atrial thrombosis, cerebral venous sinus thrombosis, thrombosis, thrombosis on papillae of an eye, deep venous thrombosis of an arm, subclavian vein thrombosis, superficial venous thrombosis of an arm, cerebral infarction, embolic stroke, or postoperative venous thrombosis. In some embodiments, the human patient has one or more risk factors for thromboembolism. In some embodiments, the risk factor for thromboembolism is persistent AT activity less than 15%, use of a fixed dose of fitusiran 80 mg monthly, presence of indwelling venous catheters, and post-operative state where bleed management guidelines are not followed.

The risk of thrombosis is multifactorial and may be greater in patients with AT activity levels <15% and/or existent comorbidities. AT levels are monitored to reduce the potential risk of thrombosis. Fitusiran is used with caution in patients with underlying conditions that may predispose them to a higher risk of thrombosis. In some embodiments, patients do not have established thrombophilic conditions or a prior history of thrombosis.

In some embodiments, the present treatment methods reduce the risk of a thrombotic event in a human patient receiving fitusiran for prophylactic treatment of hemophilia A or B with or without factor VIII or factor IX inhibitors. In some embodiments, the risk of a thrombotic event is reduced as compared to the risk of a thrombotic event in a patient treated with the original dose regimen (e.g., a regimen of 50 mg Q2M). In some embodiments, the exposure adjusted incidence rate (per 100 patient years) of thrombotic events is about 0.82 in patients treated with the AT-based dose regimen, compared to about 2.28 in patients treated with the original dose regimen.

The present treatment methods may also reduce the consumption of factor or BPA in a human patient receiving fitusiran for prophylactic treatment of hemophilia A or B with or without factor VIII or factor IX inhibitors. The reduction may be in, e.g., the annualized consumption of factor or BPA or the weight-adjusted dose of factor of BPA needed to treat a bleeding episode in the patient. In some embodiments, the methods further comprise administering an effective amount of a replacement factor or BPA to treat a bleeding episode that occurs eight or more days after the first fitusiran dose, or seven or fewer days after the first fitusiran dose. In some embodiments, if the replacement factor or BPA is administered to treat a bleeding episode that occurs eight or more days after the first fitusiran dose, the effective amount of the replacement factor or BPA is reduced as compared to the recommended effective amount of the replacement factor or BPA for human patients who are not on fitusiran therapy. This reduction can be to one half of a weight-based dose of the recommended effective amount of the replacement factor or BPA for human patients who are not on fitusiran therapy, administered at double the dosing frequency recommended for human patients who are not on fitusiran therapy. In some embodiments, if the replacement factor or BPA is administered to treat a bleeding episode that occurs seven or fewer days after the first fitusiran dose, the effective amount of the replacement factor or BPA is administered according to the human patient's prior dosing regimen of replacement factor or BPA. In yet other embodiments, the present treatment methods may also terminate prophylactic treatment with a replacement factor or BPA in a human patient before or after the first dose of fitusiran. In some embodiments, the prophylactic treatment may be terminated within about two weeks or about 14 days or about one week or about seven days of the first dose of fitusiran.

### VI. Articles of Manufacture and Kits

The present invention also provides kits comprising a pharmaceutical composition for use in the present treatment methods. Such kits include one or more vials or one or more prefilled syringes or injectors (e.g., prefilled pens such as prefilled single-use pens) comprising a pharmaceutical composition of the invention and instructions for use, e.g., instructions for administering a therapeutically effective amount of fitusiran.

In some embodiments, the pharmaceutical composition comprising fitusiran sodium (also referred to as “fitusiran drug product”) is packaged in a drug delivery device. In some embodiments, a drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. As also described in ISO 11608-1:2014(E), a single-dose container system may involve a needle-based injection device with an integrated non-replaceable container. In some embodiments, each container holds a single dose, whereby the entire deliverable volume is expelled (full evacuation). In other embodiments, each container holds a single dose, whereby a portion of the deliverable volume is expelled (partial evacuation).

An exemplary sleeve-triggered auto-injector with manual needle insertion is described in WO2015/004052. Exemplary audible end-of-dose feedback mechanisms are described in WO2016/193346 and WO2016/193348. An exemplary needle-safety mechanism after using an auto-injector is described in WO2016/193352. An exemplary needle sheath remover mechanism for a syringe auto-injector is described in WO2016/193353. An exemplary support mechanism for supporting an axial position of a syringe is described in WO2016/193355.

The pharmaceutical composition comprising fitusiran sodium may be supplied in a single-dose prefilled pen or single-use vial containing 10 mg fitusiran in 0.25 mL of solution at a concentration of 40 mg/mL. The pharmaceutical composition may be supplied in a single-dose prefilled pen or single-use vial containing 10 mg fitusiran in 0.1 mL of solution at a concentration of 100 mg/mL. The pharmaceutical composition may be supplied in a single-dose prefilled pen containing 20 mg fitusiran (equivalent to 21.2 mg fitusiran) in 0.5 mL of solution at a concentration of 40 mg/mL. The pharmaceutical composition may be supplied in a single-use vial containing 20 mg fitusiran (equivalent to 21.2 mg fitusiran sodium) in 0.2 mL of solution at a concentration of 100 mg/mL. The pharmaceutical composition may also be supplied in a single-dose prefilled pen containing 50 mg of fitusiran (equivalent to 53 mg fitusiran sodium) in 0.5 mL solution at a concentration of 100 mg/mL. In some embodiments, the pharmaceutical composition is provided as a solution for injection as 50 mg/0.5 mL (100 mg/mL) in a single-dose prefilled pen, 20 mg/0.2 mL (100 mg/mL) in a single-dose vial, 20 mg/0.5 mL (40 mg/mL) in a single-dose prefilled pen, 10 mg/0.1 mL (100 mg/mL) in a single-dose prefilled pen or single-use vial, 10 mg/0.25 mL (40 mg/mL) in a single-dose prefilled pen or single-use vial.

In some embodiments, the vial or prefilled pen is supplied in a carton. In some embodiments, the pharmaceutical composition is available in cartons containing one prefilled pen or one vial.

In some embodiments, the pharmaceutical composition comprising fitusiran sodium is a clear, colorless to pale yellow solution supplied in a single-dose prefilled pen or a single-dose vial. In some embodiments, each prefilled pen is designed to deliver 50 mg of fitusiran in 0.5 mL. In some embodiments, each vial is designed to deliver 20 mg of fitusiran in 0.2 mL. In some embodiments, each prefilled pen is designed to deliver 20 mg of fitusiran in 0.5 mL. In some embodiments, each prefilled pen or vial is designed to deliver 10 mg of fitusiran in 0.1 mL. In some embodiments, each prefilled pen or vial is designed to deliver 10 mg of fitusiran in 0.25 mL.

In some embodiments, the pharmaceutical composition is stored at 2° C. to 30° C. (36° F. to 86° F.) in the original carton to protect from light. In some embodiments, the pharmaceutical composition is not shaken, heated, frozen, or put into direct sunlight at any time.

In some embodiments, the pharmaceutical composition comprising fitusiran sodium is provided in an article of manufacture comprising a packaging material and a label or package insert contained within the packaging material indicating that serious thrombotic events can occur. In some embodiments, the pharmaceutical composition is provided in an article of manufacture comprising a packaging material and a label or package insert contained within the packaging material indicating that acute and recurrent gallbladder disease can occur. In some embodiments, the pharmaceutical composition is provided in an article of manufacture comprising a packaging material and a label or package insert contained within the packaging material indicating that hepatotoxicity can occur.

In some embodiments, the pharmaceutical composition comprising fitusiran sodium is provided in a package that further comprises a label comprising one or more messages that (a) serious thrombotic events have been reported; (b) AT levels should be monitored to reduce the potential risk of thrombosis; or (c) fitusiran prophylaxis should be interrupted if clinical symptoms, signs, or imaging findings consistent with a thrombotic event occur, and a thrombotic event should be managed as clinically indicated. In some embodiments, the pharmaceutical composition is provided in a package that further comprises a label comprising one or more messages that: (a) treatment with fitusiran has been associated with an increased occurrence of acute and recurrent gallbladder disease; or (b) if gallbladder disease occurs, consider interrupting or discontinuing fitusiran treatment. In some embodiments, fitusiran is provided in a package that further comprises a label comprising one or more messages that: (a) hepatotoxicity has been reported; (b) baseline liver function tests should be obtained and liver function should be monitored; (c) use of fitusiran should be avoided in patients with known hepatic impairment; or (d) fitusiran should be permanently discontinued if alanine aminotransferase or aspartate transaminase elevations greater than five times the upper limit of normal reoccur or the patient experiences jaundice thought to be from hepatotoxicity with other causes of liver test elevation ruled out.

In some embodiments, the pharmaceutical composition comprising fitusiran sodium is provided in a package with a label comprising a printed statement which informs a reader that the mean Cₘₐₓ of fitusiran in patients with hemophilia A and B, with or without inhibitors was 34.4 ng/mL (CV 29%) for fitusiran at 20 mg and 84.1 ng/mL (CV 70%) at 50 mg. In some embodiments, the pharmaceutical composition is provided in a package with a label comprising a printed statement which informs a reader that the mean AUC of fitusiran in patients with hemophilia A and B, with or without inhibitors was 491 ng h/mL (CV 17%) for fitusiran at 20 mg and 1290 ng h/mL (CV 29%) for fitusiran at 50 mg. In some embodiments, the pharmaceutical composition is provided in a package with a label comprising a printed statement which informs a reader that fitusiran has an apparent clearance of 41.9 CL/F (CV 20%) at 20 mg and 50.8 CL/F (CV 140%) at 50 mg.

In some embodiments, the present methods comprise promoting the use of fitusiran, and conveying to a recipient at least one message selected from: (a) serious thrombotic events have been reported; (b) AT levels should be monitored to reduce the potential risk of thrombosis; (c) fitusiran prophylaxis should be interrupted if clinical symptoms, signs, or imaging findings consistent with a thrombotic event occur, and a thrombotic event should be managed as clinically indicated; (d) treatment with fitusiran has been associated with an increased occurrence of acute and recurrent gallbladder disease; (e) if gallbladder disease occurs, consider interrupting or discontinuing fitusiran treatment; (f) hepatotoxicity has been reported; (g) baseline liver function tests should be obtained and liver function should be monitored; (h) use of fitusiran should be avoided in patients with known hepatic impairment; (i) fitusiran should be permanently discontinued if alanine aminotransferase or aspartate transaminase elevations greater than five times the upper limit of normal reoccur or the patient experiences jaundice thought to be from hepatotoxicity with other causes of liver test elevation ruled out.

In some embodiments, the present methods comprise providing fitusiran to a patient, along with information indicating that: (a) serious thrombotic events have been reported; (b) AT levels should be monitored to reduce the potential risk of thrombosis; (c) fitusiran prophylaxis should be interrupted if clinical symptoms, signs, or imaging findings consistent with a thrombotic event occur, and a thrombotic event should be managed as clinically indicated; (d) treatment with fitusiran has been associated with an increased occurrence of acute and recurrent gallbladder disease; (e) if gallbladder disease occurs, consider interrupting or discontinuing fitusiran treatment; (f) hepatotoxicity has been reported; (g) baseline liver function tests should be obtained and liver function should be monitored; (h) use of fitusiran should be avoided in patients with known hepatic impairment; or (i) fitusiran should be permanently discontinued if alanine aminotransferase or aspartate transaminase elevations greater than five times the upper limit of normal reoccur or the patient experiences jaundice thought to be from hepatotoxicity with other causes of liver test elevation ruled out.

### Exemplary Embodiments

The following embodiments are exemplary and are not intended to limit the scope of the invention or inventions described herein.

Embodiment 1. A method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising:
(a) obtaining a measurement of an antithrombin (AT) level in the human patient, wherein the human patient has previously received fitusiran at a starting dose of 50 mg about once every 2 months;
(b) performing one of the following steps:
   (i) if the AT level is 15-35%, subcutaneously administering to the human patient fitusiran at a dose of 50 mg about once every 2 months,
   (ii) if the AT level is >35% after 6 months, subcutaneously administering to the human patient fitusiran at a dose of 50 mg about once every month, or
   (iii) if the AT level is <15%, subcutaneously administering to the human patient fitusiran at a dose of 20 mg about once every 2 months;
(c) after administering to the human patient fitusiran at a dose of 20 mg about once every 2 months, obtaining a measurement of an antithrombin (AT) level in the human patient; (d) performing one of the following steps:
   (i) if the AT level is 15-35%, subcutaneously administering to the human patient fitusiran at a dose of 20 mg about once every 2 months,
   (ii) if the AT level is >35% after 6 months, subcutaneously administering to the human patient fitusiran at a dose of 20 mg about once every month, or
   (iii) if the AT level is <15%, subcutaneously administering to the human patient fitusiran at a dose of 10 mg about once every 2 months;
(e) after administering to the human patient fitusiran at a dose of 10 mg about once every 2 months, obtaining a measurement of an antithrombin (AT) level in the human patient; and
(f) performing one of the following steps:
   (i) if the AT level is 15-35%, subcutaneously administering to the human patient fitusiran at a dose of 10 mg about once every 2 months,
   (ii) if the AT level is >35% after 6 months, subcutaneously administering to the human patient fitusiran at a dose of 10 mg about once every month, or
   (iii) if the AT level is <15%, discontinuing or pausing fitusiran treatment.

Embodiment 2. A method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 20 mg about once every 2 months;
(b) obtaining a measurement of an antithrombin (AT) level in the human patient; and
(c) performing one of the following steps:
   (i) if the AT level is 15-35%, repeating step (a),
   (ii) if the AT level is >35% after 6 months, subcutaneously administering to the human patient fitusiran at a dose of 20 mg about once every month, or
   (iii) if the AT level is <15%, subcutaneously administering to the human patient fitusiran at a lower dose amount.

Embodiment 3. The method of embodiment 2, wherein step (c)(iii) comprises subcutaneously administering to the human patient fitusiran at a dose of 10 mg about once every 2 months.

Embodiment 4. A method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 10 mg about once every 2 months;
(b) obtaining a measurement of an antithrombin (AT) level in the human patient; and
(c) performing one of the following steps:
   (i) if the AT level is 15-35%, repeating step (a),
   (ii) if the AT level is >35% after 6 months, subcutaneously administering to the human patient fitusiran at a dose of 10 mg about once every month, or
   (iii) if the AT level is <15%, discontinuing or pausing fitusiran treatment.

Embodiment 5. A method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising subcutaneously administering to the human patient in need thereof fitusiran at a dose of: about 50 mg about once a month or about once every four weeks, about 20 mg about once a month or about once every four weeks, about 10 mg about once a month or about once every four weeks, about 50 mg about once every other month or about once every eight weeks, about 20 mg about once every other month or about once every eight weeks, or about 10 mg about once every other month or about once every eight weeks.

Embodiment 6. A method of reducing a risk of hepatotoxicity in a human patient with hemophilia A or B with or without factor VIII or factor IX inhibitors who receives fitusiran at a current dose for routine prophylaxis to prevent or reduce the frequency of bleeding episodes, the method comprising administering fitusiran at the current dose or a modified dose based on an antithrombin (AT) activity level in the human patient, wherein:
(a) if the current dose is 50 mg every 2 months and the AT level is <15%, fitusiran is administered at a modified dose of 20 mg every 2 months;
(b) if the current dose is 50 mg every 2 months and the AT level is 15-35%, fitusiran is administered at the current dose;
(c) if the current dose is 50 mg every 2 months and the AT level is >35% after 6 months, fitusiran is administered at a modified dose of 50 mg every month;
(d) if the current dose is 20 mg every 2 months and the AT level is <15%, fitusiran is administered at a modified dose of 10 mg every 2 months;
(e) if the current dose is 20 mg every 2 months and the AT level is 15-35%, fitusiran is administered at the current dose;
(f) if the current dose is 20 mg every 2 months and the AT level is >35% after 6 months, fitusiran is administered at a modified dose of 20 mg every month;
(g) if the current dose is 10 mg every 2 months and the AT level is <15%, fitusiran administration is discontinued;
(h) if the current dose is 10 mg every 2 months and the AT level is 15-35%, fitusiran is administered at the current dose; or
(i) if the current dose is 10 mg every 2 months and the AT level is >35% after 6 months, fitusiran is administered at a modified dose of 10 mg every month.

Embodiment 7. The method of any one of embodiments 1-6, wherein the risk of hepatotoxicity comprises a risk of alanine transaminase (ALT) elevation more than three times the upper limit of normal (ULN), aspartate aminotransferase (AST) elevation more than three times the ULN, severe liver toxicity, liver failure, and/or jaundice.

Embodiment 8. The method of embodiment 7, wherein reducing the risk of hepatotoxicity comprises reducing the occurrence in the human patient of alanine transaminase (ALT) elevation more than three times the upper limit of normal (ULN), aspartate aminotransferase (AST) elevation more than three times the ULN, severe liver toxicity, liver failure, and/or jaundice.

Embodiment 9. A method of reducing a risk of gallbladder disease in a human patient with hemophilia A or B with or without factor VIII or factor IX inhibitors who receives fitusiran for routine prophylaxis to prevent or reduce the frequency of bleeding episodes, comprising:
(a) obtaining a measurement of an antithrombin (AT) level in the human patient, wherein the human patient has previously received fitusiran at a starting dose of 50 mg about once every 2 months;
(b) performing one of the following steps:
   (i) if the AT level is 15-35%, subcutaneously administering to the human patient fitusiran at a dose of 50 mg about once every 2 months,
   (ii) if the AT level is >35% after 6 months, subcutaneously administering to the human patient fitusiran at a dose of 50 mg about once every month, or
   (iii) if the AT level is <15%, subcutaneously administering to the human patient fitusiran at a dose of 20 mg about once every 2 months;
(c) after administering to the human patient fitusiran at a dose of 20 mg about once every 2 months, obtaining a measurement of an antithrombin (AT) level in the human patient;
(d) performing one of the following steps:
   (i) if the AT level is 15-35%, subcutaneously administering to the human patient fitusiran at a dose of 20 mg about once every 2 months,
   (ii) if the AT level is >35% after 6 months, subcutaneously administering to the human patient fitusiran at a dose of 20 mg about once every month, or
   (iii) if the AT level is <15%, subcutaneously administering to the human patient fitusiran at a dose of 10 mg about once every 2 months;
(e) after administering to the human patient fitusiran at a dose of 10 mg about once every 2 months, obtaining a measurement of an antithrombin (AT) level in the human patient; and
(f) performing one of the following steps:
   (i) if the AT level is 15-35%, subcutaneously administering to the human patient fitusiran at a dose of 10 mg about once every 2 months,
   (ii) if the AT level is >35% after 6 months, subcutaneously administering to the human patient fitusiran at a dose of 10 mg about once every month, or
   (iii) if the AT level is <15%, discontinuing or pausing fitusiran treatment.

Embodiment 10. A method of reducing a risk of gallbladder disease in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 20 mg about once every 2 months;
(b) obtaining a measurement of an antithrombin (AT) level in the human patient; and
(c) performing one of the following steps:
   (i) if the AT level is 15-35%, repeating step (a),
   (ii) if the AT level is >35% after 6 months, subcutaneously administering to the human patient fitusiran at a dose of 20 mg about once every month, or
   (iii) if the AT level is <15%, subcutaneously administering to the human patient fitusiran at a lower dose amount.

Embodiment 11. The method of embodiment 10, wherein step (c)(iii) comprises subcutaneously administering to the human patient fitusiran at a dose of 10 mg about once every 2 months.

Embodiment 12. A method of reducing a risk of gallbladder disease in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 10 mg about once every 2 months;
(b) obtaining a measurement of an antithrombin (AT) level in the human patient; and
(c) performing one of the following steps:
   (i) if the AT level is 15-35%, repeating step (a),
   (ii) if the AT level is >35% after 6 months, subcutaneously administering to the human patient fitusiran at a dose of 10 mg about once every month, or
   (iii) if the AT level is <15%, discontinuing or pausing fitusiran treatment.

Embodiment 13. A method of reducing a risk of gallbladder disease in a human patient with hemophilia A or B with or without factor VIII or factor IX inhibitors who receives fitusiran for routine prophylaxis to prevent or reduce the frequency of bleeding episodes, comprising subcutaneously administering to the human patient in need thereof fitusiran at a dose of:
about 50 mg about once a month or about once every four weeks,
about 20 mg about once a month or about once every four weeks,
about 10 mg about once a month or about once every four weeks,
about 50 mg about once every other month or about once every eight weeks,
about 20 mg about once every other month or about once every eight weeks, or
about 10 mg about once every other month or about once every eight weeks.

Embodiment 14. A method of reducing a risk of gallbladder disease in a human patient with hemophilia A or B with or without factor VIII or factor IX inhibitors who receives fitusiran at a current dose for routine prophylaxis to prevent or reduce the frequency of bleeding episodes, the method comprising administering fitusiran at the current dose or a modified dose based on an antithrombin (AT) activity level in the human patient, wherein:
(a) if the current dose is 50 mg every 2 months and the AT level is <15%, fitusiran is administered at a modified dose of 20 mg every 2 months;
(b) if the current dose is 50 mg every 2 months and the AT level is 15-35%, fitusiran is administered at the current dose;
(c) if the current dose is 50 mg every 2 months and the AT level is >35% after 6 months, fitusiran is administered at a modified dose of 50 mg every month;
(d) if the current dose is 20 mg every 2 months and the AT level is <15%, fitusiran is administered at a modified dose of 10 mg every 2 months;
(e) if the current dose is 20 mg every 2 months and the AT level is 15-35%, fitusiran is administered at the current dose;
(f) if the current dose is 20 mg every 2 months and the AT level is >35% after 6 months, fitusiran is administered at a modified dose of 20 mg every month;
(g) if the current dose is 10 mg every 2 months and the AT level is <15%, fitusiran administration is discontinued;
(h) if the current dose is 10 mg every 2 months and the AT level is 15-35%, fitusiran is administered at the current dose; or
(i) if the current dose is 10 mg every 2 months and the AT level is >35% after 6 months, fitusiran is administered at a modified dose of 10 mg every month.

Embodiment 15. The method of any one of embodiments 9-14, wherein the risk of gallbladder disease comprises a risk of cholecystitis, cholelithiasis, or a need for cholecystectomy.

Embodiment 16. The method of embodiment 15, wherein reducing the risk of gallbladder disease comprises reducing the occurrence in the human patient of cholecystitis, cholelithiasis, or a need for cholecystectomy.

Embodiment 17. The method of any one of embodiments 9-16, wherein the gallbladder disease is acute and recurrent gallbladder disease.

Embodiment 18. A method of reducing thrombotic risk in a human patient with hemophilia A or B with or without factor VIII or factor IX inhibitors who receives fitusiran for routine prophylaxis to prevent or reduce the frequency of bleeding episodes, comprising:
(a) obtaining a measurement of an antithrombin (AT) level in the human patient, wherein the human patient has previously received fitusiran at a starting dose of 50 mg about once every 2 months;
(b) performing one of the following steps:
   (i) if the AT level is 15-35%, subcutaneously administering to the human patient fitusiran at a dose of 50 mg about once every 2 months,
   (ii) if the AT level is >35% after 6 months, subcutaneously administering to the human patient fitusiran at a dose of 50 mg about once every month, or
   (iii) if the AT level is <15%, subcutaneously administering to the human patient fitusiran at a dose of 20 mg about once every 2 months;
(c) after administering to the human patient fitusiran at a dose of 20 mg about once every 2 months, obtaining a measurement of an antithrombin (AT) level in the human patient; (d) performing one of the following steps:
   (i) if the AT level is 15-35%, subcutaneously administering to the human patient fitusiran at a dose of 20 mg about once every 2 months,
   (ii) if the AT level is >35% after 6 months, subcutaneously administering to the human patient fitusiran at a dose of 20 mg about once every month, or
   (iii) if the AT level is <15%, subcutaneously administering to the human patient fitusiran at a dose of 10 mg about once every 2 months;
(e) after administering to the human patient fitusiran at a dose of 10 mg about once every 2 months, obtaining a measurement of an antithrombin (AT) level in the human patient; and
(f) performing one of the following steps:
   (i) if the AT level is 15-35%, subcutaneously administering to the human patient fitusiran at a dose of 10 mg about once every 2 months,
   (ii) if the AT level is >35% after 6 months, subcutaneously administering to the human patient fitusiran at a dose of 10 mg about once every month, or
   (iii) if the AT level is <15%, discontinuing or pausing fitusiran treatment.

Embodiment 19. A method of reducing thrombotic risk in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 20 mg about once every 2 months;
(b) obtaining a measurement of an antithrombin (AT) level in the human patient; and
(c) performing one of the following steps:
   (i) if the AT level is 15-35%, repeating step (a),
   (ii) if the AT level is >35% after 6 months, subcutaneously administering to the human patient fitusiran at a dose of 20 mg about once every month, or
(iii) if the AT level is <15%, subcutaneously administering to the human patient fitusiran at a lower dose amount.

Embodiment 20. The method of embodiment 19, wherein step (c)(iii) comprises subcutaneously administering to the human patient fitusiran at a dose of 10 mg about once every 2 months.

Embodiment 21. A method of reducing thrombotic risk in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 10 mg about once every 2 months;
(b) obtaining a measurement of an antithrombin (AT) level in the human patient; and
(c) performing one of the following steps:
   (i) if the AT level is 15-35%, repeating step (a),
   (ii) if the AT level is >35% after 6 months, subcutaneously administering to the human patient fitusiran at a dose of 10 mg about once every month, or
   (iii) if the AT level is <15%, discontinuing or pausing fitusiran treatment.

Embodiment 22. A method of reducing thrombotic risk in a human patient with hemophilia A or B with or without factor VIII or factor IX inhibitors who receives fitusiran for routine prophylaxis to prevent or reduce the frequency of bleeding episodes, comprising subcutaneously administering to the human patient in need thereof fitusiran at a dose of:
about 50 mg about once a month or about once every four weeks,
about 20 mg about once a month or about once every four weeks,
about 10 mg about once a month or about once every four weeks,
about 50 mg about once every other month or about once every eight weeks,
about 20 mg about once every other month or about once every eight weeks, or
about 10 mg about once every other month or about once every eight weeks.

Embodiment 23. A method of reducing thrombotic risk in a human patient with hemophilia A or B with or without factor VIII or factor IX inhibitors who receives fitusiran at a current dose for routine prophylaxis to prevent or reduce the frequency of bleeding episodes, the method comprising administering fitusiran at the current dose or a modified dose based on an antithrombin (AT) activity level in the human patient, wherein:
(a) if the current dose is 50 mg every 2 months and the AT level is <15%, fitusiran is administered at a modified dose of 20 mg every 2 months;
(b) if the current dose is 50 mg every 2 months and the AT level is 15-35%, fitusiran is administered at the current dose;
(c) if the current dose is 50 mg every 2 months and the AT level is >35% after 6 months, fitusiran is administered at a modified dose of 50 mg every month;
(d) if the current dose is 20 mg every 2 months and the AT level is <15%, fitusiran is administered at a modified dose of 10 mg every 2 months;
(e) if the current dose is 20 mg every 2 months and the AT level is 15-35%, fitusiran is administered at the current dose;
(f) if the current dose is 20 mg every 2 months and the AT level is >35% after 6 months, fitusiran is administered at a modified dose of 20 mg every month;
(g) if the current dose is 10 mg every 2 months and the AT level is <15%, fitusiran administration is discontinued;
(h) if the current dose is 10 mg every 2 months and the AT level is 15-35%, fitusiran is administered at the current dose; or
(i) if the current dose is 10 mg every 2 months and the AT level is >35% after 6 months, fitusiran is administered at a modified dose of 10 mg every month.

Embodiment 24. The method of any one of embodiments 18-23, wherein the thrombotic risk comprises a risk of a serious thrombotic event.

Embodiment 25. The method of embodiment 24, wherein the serious thrombotic event comprises cerebrovascular accident, cerebral artery embolism, spinal vascular disorder, atrial thrombosis, cerebral venous sinus thrombosis, thrombosis, thrombosis on papillae of an eye, deep venous thrombosis of an arm, subclavian vein thrombosis, superficial venous thrombosis of an arm, cerebral infarction, embolic stroke, or postoperative venous thrombosis.

Embodiment 26. The method of embodiment 24 or embodiment 25, wherein reducing thrombotic risk comprises reducing the occurrence in the human patient of cerebrovascular accident, cerebral artery embolism, spinal vascular disorder, atrial thrombosis, cerebral venous sinus thrombosis, thrombosis, thrombosis on papillae of an eye, deep venous thrombosis of an arm, subclavian vein thrombosis, superficial venous thrombosis of an arm, cerebral infarction, embolic stroke, or postoperative venous thrombosis.

Embodiment 27. The method of any one of embodiments 18-26, wherein the human patient has one or more risk factors for thromboembolism.

Embodiment 28. The method of embodiment 27, wherein the risk factor for thromboembolism is persistent AT activity less than 15%, use of a fixed dose of fitusiran 80 mg monthly, presence of indwelling venous catheters, and post-operative state where bleed management guidelines are not followed.

Embodiment 29. A method of routine prophylaxis to prevent or reduce the frequency of bleeding episodes in a human patient having hemophilia A or B with or without factor VIII or factor IX inhibitors, comprising:
(a) subcutaneously administering to the human patient in need thereof fitusiran at a starting dose of 50 mg every 2 months;
(b) obtaining a measurement of an antithrombin (AT) level in the human patient;
(c) performing one of the following steps:
   (i) if the AT level is 15-35%, repeating step (a),
   (ii) if the AT level is >35% after 6 months, subcutaneously administering to the human patient fitusiran at a dose of 50 mg about once every month, or
   (iii) if the AT level is <15%, subcutaneously administering to the human patient fitusiran at a dose of 20 mg about once every 2 months;
(d) after administering to the human patient fitusiran at a dose of 20 mg every 2 months, obtaining a measurement of an antithrombin (AT) level in the human patient;
(e) performing one of the following steps:
   (i) if the AT level is 15-35%, subcutaneously administering to the human patient fitusiran at a dose of 20 mg about once every 2 months,
   (ii) if the AT level is >35% after 6 months, subcutaneously administering to the human patient fitusiran at a dose of 20 mg about once every month, or
   (iii) if the AT level is <15%, subcutaneously administering to the human patient fitusiran at a dose of 10 mg about once every 2 months;
(f) after administering to the human patient fitusiran at a dose of 10 mg about once every 2 months, obtaining a measurement of an antithrombin (AT) level in the human patient; and
(g) performing one of the following steps:
   (i) if the AT level is 15-35%, subcutaneously administering to the human patient fitusiran at a dose of 10 mg about once every 2 months,
   (ii) if the AT level is >35% after 6 months, subcutaneously administering to the human patient fitusiran at a dose of 10 mg about once every month, or
   (iii) if the AT level is <15%, discontinuing or pausing fitusiran treatment.

Embodiment 30. A method of routine prophylaxis to prevent or reduce the frequency of bleeding episodes in a human patient having hemophilia A or B with or without factor VIII or factor IX inhibitors, comprising:
(a) subcutaneously administering to the human patient in need thereof fitusiran at a dose of 20 mg about once every 2 months;
(b) obtaining a measurement of an antithrombin (AT) level in the human patient; and
(c) performing one of the following steps:
   (i) if the AT level is 15-35%, repeating step (a),
   (ii) if the AT level is >35% after 6 months, subcutaneously administering to the human patient fitusiran at a dose of 20 mg about once every month, or
   (iii) if the AT level is <15%, subcutaneously administering to the human patient fitusiran at a lower dose amount.

Embodiment 31. The method of embodiment 30, wherein step (c)(iii) comprises subcutaneously administering to the human patient fitusiran at a dose of 10 mg about once every 2 months.

Embodiment 32. A method of routine prophylaxis to prevent or reduce the frequency of bleeding episodes in a human patient having hemophilia A or B with or without factor VIII or factor IX inhibitors, comprising:
(a) subcutaneously administering to the human patient in need thereof fitusiran at a dose of 10 mg about once every 2 months;
(b) obtaining a measurement of an antithrombin (AT) level in the human patient; and
(c) performing one of the following steps:
   (i) if the AT level is 15-35%, repeating step (a),
   (ii) if the AT level is >35% after 6 months, subcutaneously administering to the human patient fitusiran at a dose of 10 mg about once every month, or
   (iii) if the AT level is <15%, discontinuing or pausing fitusiran treatment.

Embodiment 33. A method of routine prophylaxis to prevent or reduce the frequency of bleeding episodes in a human patient having hemophilia A or B with or without factor VIII or factor IX inhibitors, comprising subcutaneously administering to the human patient in need thereof fitusiran at a dose of:
about 50 mg about once a month or about once every four weeks,
about 20 mg about once a month or about once every four weeks,
about 10 mg about once a month or about once every four weeks,
about 50 mg about once every other month or about once every eight weeks,
about 20 mg about once every other month or about once every eight weeks, or
about 10 mg about once every other month or about once every eight weeks.

Embodiment 34. A method of routine prophylaxis to prevent or reduce the frequency of bleeding episodes in a human patient having hemophilia A or B with or without factor VIII or factor IX inhibitors, the method comprising administering fitusiran at a first dose or a modified dose based on an antithrombin (AT) activity level in the human patient, wherein: (a) if the first dose is 50 mg every 2 months and the AT level is <15%, fitusiran is administered at a modified dose of 20 mg every 2 months;
(b) if the first dose is 50 mg every 2 months and the AT level is 15-35%, fitusiran is administered again at the first dose;
(c) if the first dose is 50 mg every 2 months and the AT level is >35% after 6 months, fitusiran is administered at a modified dose of 50 mg every month;
(d) if the first dose is 20 mg every 2 months and the AT level is <15%, fitusiran is administered at a modified dose of 10 mg every 2 months;
(e) if the first dose is 20 mg every 2 months and the AT level is 15-35%, fitusiran is administered again at the first dose;
(f) if the first dose is 20 mg every 2 months and the AT level is >35% after 6 months, fitusiran is administered at a modified dose of 20 mg every month;
(g) if the first dose is 10 mg every 2 months and the AT level is <15%, fitusiran administration is discontinued;
(h) if the first dose is 10 mg every 2 months and the AT level is 15-35%, fitusiran is administered again at the first dose; or
(i) if the first dose is 10 mg every 2 months and the AT level is >35% after 6 months, fitusiran is administered at a modified dose of 10 mg every month.

Embodiment 35. A method of monitoring an antithrombin level in a human patient with hemophilia A or B with or without factor VIII or factor IX inhibitors who receives fitusiran for routine prophylaxis to prevent or reduce the frequency of bleeding episodes, comprising:
(a) obtaining a measurement of an antithrombin (AT) level in the human patient, wherein the human patient has previously received fitusiran at a starting dose of 50 mg about once every 2 months;
(b) performing one of the following steps:
   (i) if the AT level is 15-35%, subcutaneously administering to the human patient fitusiran at a dose of 50 mg about once every 2 months,
   (ii) if the AT level is >35% after 6 months, subcutaneously administering to the human patient fitusiran at a dose of 50 mg about once every month, or
   (iii) if the AT level is <15%, subcutaneously administering to the human patient fitusiran at a dose of 20 mg about once every 2 months;
(c) after administering to the human patient fitusiran at a dose of 20 mg about once every 2 months, obtaining a measurement of an antithrombin (AT) level in the human patient;
(d) performing one of the following steps:
   (i) if the AT level is 15-35%, subcutaneously administering to the human patient fitusiran at a dose of 20 mg about once every 2 months,
   (ii) if the AT level is >35% after 6 months, subcutaneously administering to the human patient fitusiran at a dose of 20 mg about once every month, or
   (iii) if the AT level is <15%, subcutaneously administering to the human patient fitusiran at a dose of 10 mg about once every 2 months;
(e) after administering to the human patient fitusiran at a dose of 10 mg about once every 2 months, obtaining a measurement of an antithrombin (AT) level in the human patient; and
(f) performing one of the following steps:
   (i) if the AT level is 15-35%, subcutaneously administering to the human patient fitusiran at a dose of 10 mg about once every 2 months,
   (ii) if the AT level is >35% after 6 months, subcutaneously administering to the human patient fitusiran at a dose of 10 mg about once every month, or
   (iii) if the AT level is <15%, discontinuing or pausing fitusiran treatment.

Embodiment 36. A method of monitoring an antithrombin level in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 20 mg about once every 2 months;
(b) obtaining a measurement of an antithrombin (AT) level in the human patient; and
(c) performing one of the following steps:
   (i) if the AT level is 15-35%, repeating step (a),
   (ii) if the AT level is >35% after 6 months, subcutaneously administering to the human patient fitusiran at a dose of 20 mg about once every month, or
   (iii) if the AT level is <15%, subcutaneously administering to the human patient fitusiran at a lower dose amount.

Embodiment 37. The method of embodiment 36, wherein step (c)(iii) comprises subcutaneously administering to the human patient fitusiran at a dose of 10 mg about once every 2 months.

Embodiment 38. A method of monitoring an antithrombin level in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 10 mg about once every 2 months;
(b) obtaining a measurement of an antithrombin (AT) level in the human patient; and
(c) performing one of the following steps:
   (i) if the AT level is 15-35%, repeating step (a),
   (ii) if the AT level is >35% after 6 months, subcutaneously administering to the human patient fitusiran at a dose of 10 mg about once every month, or
   (iii) if the AT level is <15%, discontinuing or pausing fitusiran treatment.

Embodiment 39. A method of monitoring an antithrombin level in a human patient with hemophilia A or B with or without factor VIII or factor IX inhibitors who receives fitusiran for routine prophylaxis to prevent or reduce the frequency of bleeding episodes, comprising subcutaneously administering to the human patient in need thereof fitusiran at a dose of:
about 50 mg about once a month or about once every four weeks,
about 20 mg about once a month or about once every four weeks,
about 10 mg about once a month or about once every four weeks,
about 50 mg about once every other month or about once every eight weeks,
about 20 mg about once every other month or about once every eight weeks, or
about 10 mg about once every other month or about once every eight weeks.

Embodiment 40. A method of monitoring an antithrombin level in a human patient with hemophilia A or B with or without factor VIII or factor IX inhibitors who receives fitusiran at a current dose for routine prophylaxis to prevent or reduce the frequency of bleeding episodes, the method comprising administering fitusiran at the current dose or a modified dose based on an antithrombin (AT) activity level in the human patient, wherein:
(a) if the current dose is 50 mg every 2 months and the AT level is <15%, fitusiran is administered at a modified dose of 20 mg every 2 months;
(b) if the current dose is 50 mg every 2 months and the AT level is 15-35%, fitusiran is administered at the current dose;
(c) if the current dose is 50 mg every 2 months and the AT level is >35% after 6 months, fitusiran is administered at a modified dose of 50 mg every month;
(d) if the current dose is 20 mg every 2 months and the AT level is <15%, fitusiran is administered at a modified dose of 10 mg every 2 months;
(e) if the current dose is 20 mg every 2 months and the AT level is 15-35%, fitusiran is administered at the current dose;
(f) if the current dose is 20 mg every 2 months and the AT level is >35% after 6 months, fitusiran is administered at a modified dose of 20 mg every month;
(g) if the current dose is 10 mg every 2 months and the AT level is <15%, fitusiran administration is discontinued;
(h) if the current dose is 10 mg every 2 months and the AT level is 15-35%, fitusiran is administered at the current dose; or
(i) if the current dose is 10 mg every 2 months and the AT level is >35% after 6 months, fitusiran is administered at a modified dose of 10 mg every month.

Embodiment 41. The method of any one of embodiments 35-40, wherein the antithrombin level is monitored using a kinetic or chromogenic assay.

Embodiment 42. The method of embodiment 41, wherein the antithrombin level is monitored using a chromogenic assay that quantifies functionally active AT in human citrated plasma based on the inhibition of an excess of factor Xa by AT.

Embodiment 43. The method of embodiment 42, wherein the chromogenic assay is an INNOVANCE™ Antithrombin assay.

Embodiment 44. A method of reducing annualized bleeding rate in a human patient with hemophilia A or B with or without factor VIII or factor IX inhibitors who receives fitusiran for routine prophylaxis to prevent or reduce the frequency of bleeding episodes, comprising:
(a) obtaining a measurement of an antithrombin (AT) level in the human patient, wherein the human patient has previously received fitusiran at a starting dose of 50 mg about once every 2 months;
(b) performing one of the following steps:
   (i) if the AT level is 15-35%, subcutaneously administering to the human patient fitusiran at a dose of 50 mg about once every 2 months,
   (ii) if the AT level is >35% after 6 months, subcutaneously administering to the human patient fitusiran at a dose of 50 mg about once every month, or
   (iii) if the AT level is <15%, subcutaneously administering to the human patient fitusiran at a dose of 20 mg about once every 2 months;
(c) after administering to the human patient fitusiran at a dose of 20 mg about once every 2 months, obtaining a measurement of an antithrombin (AT) level in the human patient;
(d) performing one of the following steps:
   (i) if the AT level is 15-35%, subcutaneously administering to the human patient fitusiran at a dose of 20 mg about once every 2 months,
   (ii) if the AT level is >35% after 6 months, subcutaneously administering to the human patient fitusiran at a dose of 20 mg about once every month, or
   (iii) if the AT level is <15%, subcutaneously administering to the human patient fitusiran at a dose of 10 mg about once every 2 months;
(e) after administering to the human patient fitusiran at a dose of 10 mg about once every 2 months, obtaining a measurement of an antithrombin (AT) level in the human patient; and
(f) performing one of the following steps:
   (i) if the AT level is 15-35%, subcutaneously administering to the human patient fitusiran at a dose of 10 mg about once every 2 months,
   (ii) if the AT level is >35% after 6 months, subcutaneously administering to the human patient fitusiran at a dose of 10 mg about once every month, or
   (iii) if the AT level is <15%, discontinuing or pausing fitusiran treatment.

Embodiment 45. A method of reducing annualized bleeding rate in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 20 mg about once every 2 months;
(b) obtaining a measurement of an antithrombin (AT) level in the human patient; and
(c) performing one of the following steps:
   (i) if the AT level is 15-35%, repeating step (a),
   (ii) if the AT level is >35% after 6 months, subcutaneously administering to the human patient fitusiran at a dose of 20 mg about once every month, or
   (iii) if the AT level is <15%, subcutaneously administering to the human patient fitusiran at a lower dose amount.

Embodiment 46. The method of embodiment 45, wherein step (c)(iii) comprises subcutaneously administering to the human patient fitusiran at a dose of 10 mg about once every 2 months.

Embodiment 47. A method of reducing annualized bleeding rate in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 10 mg about once every 2 months;
(b) obtaining a measurement of an antithrombin (AT) level in the human patient; and
(c) performing one of the following steps:
   (i) if the AT level is 15-35%, repeating step (a),
   (ii) if the AT level is >35% after 6 months, subcutaneously administering to the human patient fitusiran at a dose of 10 mg about once every month, or
   (iii) if the AT level is <15%, discontinuing or pausing fitusiran treatment.

Embodiment 48. A method of reducing annualized bleeding rate in a human patient with hemophilia A or B with or without factor VIII or factor IX inhibitors who receives fitusiran for routine prophylaxis to prevent or reduce the frequency of bleeding episodes, comprising subcutaneously administering to the human patient in need thereof fitusiran at a dose of:
about 50 mg about once a month or about once every four weeks,
about 20 mg about once a month or about once every four weeks,
about 10 mg about once a month or about once every four weeks,
about 50 mg about once every other month or about once every eight weeks,
about 20 mg about once every other month or about once every eight weeks, or
about 10 mg about once every other month or about once every eight weeks.

Embodiment 49. A method of reducing annualized bleeding rate in a human patient with hemophilia A or B with or without factor VIII or factor IX inhibitors who receives fitusiran at a current dose for routine prophylaxis to prevent or reduce the frequency of bleeding episodes, the method comprising administering fitusiran at the current dose or a modified dose based on an antithrombin (AT) activity level in the human patient, wherein:
(a) if the current dose is 50 mg every 2 months and the AT level is <15%, fitusiran is administered at a modified dose of 20 mg every 2 months;
(b) if the current dose is 50 mg every 2 months and the AT level is 15-35%, fitusiran is administered at the current dose;
(c) if the current dose is 50 mg every 2 months and the AT level is >35% after 6 months, fitusiran is administered at a modified dose of 50 mg every month;
(d) if the current dose is 20 mg every 2 months and the AT level is <15%, fitusiran is administered at a modified dose of 10 mg every 2 months;
(e) if the current dose is 20 mg every 2 months and the AT level is 15-35%, fitusiran is administered at the current dose;
(f) if the current dose is 20 mg every 2 months and the AT level is >35% after 6 months, fitusiran is administered at a modified dose of 20 mg every month;
(g) if the current dose is 10 mg every 2 months and the AT level is <15%, fitusiran administration is discontinued;
(h) if the current dose is 10 mg every 2 months and the AT level is 15-35%, fitusiran is administered at the current dose; or
(i) if the current dose is 10 mg every 2 months and the AT level is >35% after 6 months, fitusiran is administered at a modified dose of 10 mg every month.

Embodiment 50. The method of any one of the above embodiments, further comprising administering an effective amount of a replacement factor or bypassing agent (BPA) to treat a bleeding episode that occurs eight or more days after the first fitusiran dose, wherein the effective amount of the replacement factor or BPA is reduced as compared to the recommended effective amount of the replacement factor or BPA for human patients who are not on fitusiran therapy.

Embodiment 51. The method of embodiment 50, wherein the effective amount of the replacement factor or BPA is reduced to one half of a weight-based dose of the recommended effective amount of the replacement factor or BPA for human patients who are not on fitusiran therapy, administered at double the dosing frequency recommended for human patients who are not on fitusiran therapy.

Embodiment 52. The method of any one of embodiments 1-51, wherein the human patient is a hemophilia A patient without factor VIII inhibitors.

Embodiment 53. The method of embodiment 52, wherein the replacement factor is factor VIII (FVIII) and a single dose of FVIII is no more than 20 IU/kg.

Embodiment 54. The method of embodiment 53, wherein the single dose of FVIII is 10 IU/kg.

Embodiment 55. The method of embodiment 53 or embodiment 54, wherein the FVIII administration is repeated, if needed, in no less than 24 hours.

Embodiment 56. The method of any one of embodiments 1-51, wherein the human patient is a hemophilia B patient without factor IX inhibitors.

Embodiment 57. The method of embodiment 56, wherein the replacement factor is factor IX (FIX) and a single dose of FIX is no more than 30 IU/kg.

Embodiment 58. The method of embodiment 57, wherein the single dose of FIX is 20 IU/kg.

Embodiment 59. The method of embodiment 57 or embodiment 58, wherein the Factor IX administration is repeated, if needed, in no less than 24 hours for standard half-life FIX or in no less than 5-7 days for extended half-life FIX.

Embodiment 60. The method of any one of embodiments 1-51, wherein the human patient is a hemophilia A patient with factor VIII inhibitors.

Embodiment 61. The method of any one of embodiments 1-51, wherein the human patient is a hemophilia B patient with factor IX inhibitors.

Embodiment 62. The method of embodiment 60 or embodiment 61, wherein the BPA is activated prothrombin complex concentrate (aPCC) and a single dose of aPCC is no more than 50 U/kg.

Embodiment 63. The method of embodiment 62, wherein the single dose of aPCC is 30 U/kg.

Embodiment 64. The method of embodiment 62 or embodiment 63, wherein the aPCC administration is repeated, if needed, in no less than 24 hours.

Embodiment 65. The method of embodiment 60 or embodiment 61, wherein the BPA is recombinant factor VIIa (rFVIIa) and a single dose of rFVIIa is no more than 45 g/kg.

Embodiment 66. The method of embodiment 65, wherein the rFVIIa administration is repeated, if needed, in no less than two hours.

Embodiment 67. The method of any one of embodiments 1-49, further comprising administering an effective amount of a replacement factor or bypassing agent (BPA) to treat a bleeding episode that occurs seven or fewer days after the first fitusiran dose, wherein the effective amount of the replacement factor or BPA is administered according to the human patient's prior dosing regimen of replacement factor or BPA.

Embodiment 68. The method of embodiment 67, wherein the human patient is a hemophilia A patient without factor VIII inhibitors.

Embodiment 69. The method of embodiment 67, wherein the human patient is a hemophilia B patient without factor IX inhibitors.

Embodiment 70. The method of embodiments 67, wherein the human patient is a hemophilia A patient with factor VIII inhibitors.

Embodiment 71. The method of embodiment 67, wherein the human patient is a hemophilia B patient with factor IX inhibitors.

Embodiment 72. The method of any one of embodiments 1-71, wherein the bleeding episode is a major surgery.

Embodiment 73. The method of embodiment 72, wherein the human patient is a patient not being treated with AT replacement therapy before, during, or after surgery.

Embodiment 74. The method of embodiment 72 or embodiment 73, wherein the major surgery is an opening into a major body cavity, operation on a joint, removal of an organ, operative alteration of normal anatomy, crossing of a mesenchymal barrier, dental extraction of molar teeth or >3 nonmolar teeth, or tooth implantation.

Embodiment 75. The method of any one of embodiments 1-74, wherein the human patient has been on prophylactic treatment with a replacement factor or a bypassing agent (BPA), and wherein the method comprises terminating the prophylactic replacement factor or BPA treatment in the human patient within about two weeks or about 14 days or about one week or about seven days of the first dose of fitusiran.

Embodiment 76. The method of any one of embodiments 1-75, comprising obtaining a measurement of AT level in the human patient about every four weeks, about every eight weeks, about every month, about every two months, about every four months, about every six months, or about every 12 months.

Embodiment 77. The method of any one of embodiments 1-75, comprising obtaining a measurement of AT level in the human patient at four weeks or one month, twelve weeks or three months, 20 weeks or five months, and 24 weeks or six months following administration of the starting dose or following administration of a modified dose as compared to the prior dose.

Embodiment 78. The method of any one of embodiments 1-77, further comprising obtaining a second measurement of AT level if the AT level is <15%.

Embodiment 79. The method of embodiment 78, further comprising subcutaneously administering to the human patient fitusiran at a lower dose amount 3 months after the prior dose if the second measurement of AT level is <15%.

Embodiment 80. The method of embodiment 78, further comprising discontinuing or pausing fitusiran treatment if the second measurement of AT level is <15%.

Embodiment 81. The method of embodiment 79 or embodiment 80, further comprising obtaining a measurement of AT level at four weeks or one month, twelve weeks or three months, 20 weeks or five months, and 24 weeks or six months after a dose reduction in fitusiran.

Embodiment 82. The method of any one of embodiments 1-77, further comprising administering to the human patient fitusiran at a higher dose amount if the AT level is >35% after six months.

Embodiment 83. The method of embodiment 77, further comprising administering to the human patient fitusiran at a higher dose amount if the human patient has not achieved satisfactory bleed control.

Embodiment 84. The method of embodiment 82 or embodiment 83, further comprising obtaining a measurement of AT level at four weeks or one month, twelve weeks or three months, 20 weeks or five months, and 24 weeks or six months after a dose escalation in fitusiran.

Embodiment 85. The method of any one of embodiments 1-84, further comprising obtaining a subsequent measurement of AT level about every 12 months if the AT level is 15-35%.

Embodiment 86. The method of any one of embodiments 1-85, wherein the obtaining a measurement of the AT level in the human patient comprises use of a kinetic or chromogenic assay.

Embodiment 87. The method of embodiment 86, wherein obtaining a measurement of the AT level in the human patient comprises use of a chromogenic assay that quantifies functionally active AT in human citrated plasma based on the inhibition of an excess of factor Xa by AT.

Embodiment 88. The method of embodiment 87, wherein obtaining a measurement of the AT level in the human patient comprises use of an INNOVANCE™ Antithrombin assay.

Embodiment 89. The method of any one of embodiments 1-88, further comprising subcutaneously administering fitusiran to the human patient at a dose amount and a dosing frequency sufficient to maintain the AT level in the human patient at 15-35%.

Embodiment 90. The method of embodiment 89, further comprising subcutaneously administering fitusiran at:
10 mg QM or Q4W,
10 mg Q2M or Q8W,
20 mg QM or Q4W,
20 mg Q2M or Q8W,
50 mg QM or Q4W, or
50 mg Q2M or Q8W.

Embodiment 91. The method of any one of embodiments 1-90, further comprising obtaining a liver function test in the human patient (i) prior to administering the starting dose of fitusiran to determine a baseline level of liver function; and (ii) about every month following initiation of fitusiran treatment.

Embodiment 92. The method of embodiment 91, wherein the liver function test is an aspartate transaminase test, an alanine aminotransferase test, and/or a measurement of total bilirubin.

Embodiment 93. The method of embodiment 92, wherein the liver function test is obtained about every month for at least the first six months following initiation of fitusiran treatment.

Embodiment 94. The method of embodiment 93, further comprising discontinuing or pausing fitusiran treatment if the aspartate transaminase or alanine aminotransferase test shows aspartate transaminase or alanine aminotransferase levels that are progressing or are persistent and five times the upper limit of normal or if the total bilirubin is >2.5 mg/dL.

Embodiment 95. The method of embodiment 94, wherein fitusiran treatment is resumed if the aspartate transaminase or alanine aminotransferase test shows aspartate transaminase or alanine aminotransferase levels have returned to baseline level and if the total bilirubin level has returned to baseline.

Embodiment 96. The method of any one of embodiments 1-95, further comprising discontinuing or pausing fitusiran treatment if the human patient is diagnosed with acute and recurrent gallbladder disease.

Embodiment 97. The method of embodiment 96, wherein the acute and recurrent gallbladder disease comprises cholecystitis, cholelithiasis, or a need for cholecystectomy.

Embodiment 98. The method of any one of embodiments 1-97, wherein the human patient does not have
(i) clinically significant liver disease,
(ii) ALT >1.5× upper limit of normal reference range (ULN),
(iii) AST >1.5× upper limit of normal reference range (ULN),
(iv) hepatitis C,
(v) hepatitis A,
(vi) hepatitis E,
(vii) hepatitis B,
(viii) acute and recurrent gallbladder disease,
(ix) symptomatic gallbladder disease,
(x) established thrombophilic conditions,
(xi) a prior history of thrombosis,
(xii) an indwelling venous catheter,
(xiii) persistent AT activity <15%, or
(xiv) known hepatic impairment.

Embodiment 99. The method of embodiment 98, wherein the known hepatic impairment is Child-Pugh Class A, B, or C.

Embodiment 100. The method of any one of embodiments 1-99, wherein the human patient is an adult or adolescent patient twelve years or older.

Embodiment 101. The method of any one of embodiments 1-100, wherein fitusiran is provided in a phosphate-buffered saline (PBS) at about 40-200 mg/mL.

Embodiment 102. The method of embodiment 101, wherein fitusiran is provided in a PBS at about 40 mg/mL or about 100 mg/mL.

Embodiment 103. The method of embodiment 101 or embodiment 102, wherein the PBS has a pH of 7.

Embodiment 104. Fitusiran for use in a method of any one of embodiments 1-103.

Embodiment 105. Use of fitusiran in the manufacture of a medicament for use in a method of any one of embodiments 1-103.

Embodiment 106. A pharmaceutical composition comprising fitusiran for use in a method of any one of embodiments 1-103.

Embodiment 107. An article of manufacture for use in a method of any one of embodiments 1-103.

Embodiment 108. The article of manufacture for use of embodiment 107, wherein the article of manufacture is a kit.

Embodiment 109. The article of manufacture for use of embodiment 107 or embodiment 108, wherein the article of manufacture is a container containing one or more doses of fitusiran, each dose being 50 mg, 20 mg, or 10 mg.

Embodiment 110. The article of manufacture for use of embodiment 109, wherein
the 50 mg of fitusiran is in 0.5 mL of a PBS,
the 20 mg of fitusiran is in 0.2 mL of a PBS,
the 20 mg of fitusiran is in 0.5 mL of a PBS,
the 10 mg of fitusiran is in 0.25 mL of a PBS, or
the 10 mg of fitusiran is in 0.1 mL of a PBS.

Embodiment 111. The article of manufacture for use of embodiment 110, wherein the PBS has a pH of 7.

Embodiment 112. The article of manufacture for use of embodiment 111, wherein the container is a single-use, single-dose prefilled syringe.

Embodiment 113. The article of manufacture for use of embodiment 111, wherein the container is a single-use glass vial.

Embodiment 114. An article of manufacture comprising:
(a) a packaging material;
(b) fitusiran; and
(c) a label or package insert contained within the packaging material indicating that serious thrombotic events can occur.

Embodiment 115. An article of manufacture comprising:
(a) a packaging material;
(b) fitusiran; and
(c) a label or package insert contained within the packaging material indicating that acute and recurrent gallbladder disease can occur.

Embodiment 116. An article of manufacture comprising:
(a) a packaging material;
(b) fitusiran; and
(c) a label or package insert contained within the packaging material indicating that hepatotoxicity can occur.

Embodiment 117. A package comprising fitusiran and a label, said label comprising one or more messages that:
(a) serious thrombotic events have been reported;
(b) AT levels should be monitored to reduce the potential risk of thrombosis; or
(c) fitusiran prophylaxis should be interrupted if clinical symptoms, signs, or imaging findings consistent with a thrombotic event occur, and a thrombotic event should be managed as clinically indicated.

Embodiment 118. A package comprising fitusiran and a label, said label comprising one or more messages that:
(a) treatment with fitusiran has been associated with an increased occurrence of acute and recurrent gallbladder disease; or
(b) if gallbladder disease occurs, consider interrupting or discontinuing fitusiran treatment.

Embodiment 119. A package comprising fitusiran and a label, said label comprising one or more messages that:
(a) hepatotoxicity has been reported;
(b) baseline liver function tests should be obtained and liver function should be monitored;
(c) use of fitusiran should be avoided in patients with known hepatic impairment; or
(d) fitusiran should be permanently discontinued if alanine aminotransferase or aspartate transaminase elevations greater than five times the upper limit of normal reoccur or the patient experiences jaundice thought to be from hepatotoxicity with other causes of liver test elevation ruled out.

Embodiment 120. A package comprising fitusiran and a label, said label comprising a printed statement which informs a reader that the mean Cₘₐₓ of fitusiran in patients with hemophilia A and B, with or without inhibitors was 34.4 ng/mL (CV 29%) for fitusiran at 20 mg and 84.1 ng/mL (CV 70%) at 50 mg.

Embodiment 121. A package comprising fitusiran and a label, said label comprising a printed statement which informs a reader that the mean AUC of fitusiran in patients with hemophilia A and B, with or without inhibitors was 491 ng h/mL (CV 17%) for fitusiran at 20 mg and 1290 ng h/mL (CV 29%) for fitusiran at 50 mg.

Embodiment 122. A package comprising fitusiran and a label, said label comprising a printed statement which informs a reader that fitusiran has an apparent clearance of 41.9 CL/F (CV 20%) at 20 mg and 50.8 CL/F (CV 140%) at 50 mg.

Embodiment 123. A method of promoting the use of fitusiran, the method comprising the step of conveying to a recipient at least one message selected from:
(a) serious thrombotic events have been reported;
(b) AT levels should be monitored to reduce the potential risk of thrombosis;
(c) fitusiran prophylaxis should be interrupted if clinical symptoms, signs, or imaging findings consistent with a thrombotic event occur, and a thrombotic event should be managed as clinically indicated;
(d) treatment with fitusiran has been associated with an increased occurrence of acute and recurrent gallbladder disease;
(e) if gallbladder disease occurs, consider interrupting or discontinuing fitusiran treatment;
(f) hepatotoxicity has been reported;
(g) baseline liver function tests should be obtained and liver function should be monitored;
(h) use of fitusiran should be avoided in patients with known hepatic impairment;
(i) fitusiran should be permanently discontinued if alanine aminotransferase or aspartate transaminase elevations greater than five times the upper limit of normal reoccur or the patient experiences jaundice thought to be from hepatotoxicity with other causes of liver test elevation ruled out.

Embodiment 124. A method of providing fitusiran, wherein said fitusiran is provided along with information indicating that:
(a) serious thrombotic events have been reported;
(b) AT levels should be monitored to reduce the potential risk of thrombosis;
(c) fitusiran prophylaxis should be interrupted if clinical symptoms, signs, or imaging findings consistent with a thrombotic event occur, and a thrombotic event should be managed as clinically indicated;
(d) treatment with fitusiran has been associated with an increased occurrence of acute and recurrent gallbladder disease;
(e) if gallbladder disease occurs, consider interrupting or discontinuing fitusiran treatment;
(f) hepatotoxicity has been reported;
(g) baseline liver function tests should be obtained and liver function should be monitored;
(h) use of fitusiran should be avoided in patients with known hepatic impairment; or
(i) fitusiran should be permanently discontinued if alanine aminotransferase or aspartate transaminase elevations greater than five times the upper limit of normal reoccur or the patient experiences jaundice thought to be from hepatotoxicity with other causes of liver test elevation ruled out.

Embodiment 125. A pharmaceutical composition, comprising fitusiran at a concentration of about 35 mg/ml to about 45 mg/mL and phosphate buffered saline (PBS) at a concentration of about 1 mM to about 10 mM, wherein the pH and the osmolality of the pharmaceutical composition are suitable for subcutaneous administration to a subject.

Embodiment 126. The pharmaceutical composition of embodiment 125, wherein the concentration of PBS is about 3 to about 6 mM.

Embodiment 127. The pharmaceutical composition of embodiment 126, wherein the concentration of PBS is about 5 mM.

Embodiment 128. The pharmaceutical composition of any one of embodiments 125-127, wherein the concentration of the fitusiran in the pharmaceutical composition is about 40 mg/mL.

Embodiment 129. The pharmaceutical composition of any one of embodiments 125-128, wherein the composition comprises sodium chloride, dibasic sodium phosphate (heptahydrate), and monobasic sodium phosphate.

Embodiment 130. The pharmaceutical composition of embodiment 129, wherein the sodium chloride is about 7.68 to 7.69 mg/mL.

Embodiment 131. The pharmaceutical composition of embodiment 129 or embodiment 130, wherein the dibasic sodium phosphate (heptahydrate) is about 0.932 to 0.934 mg/mL.

Embodiment 132. The pharmaceutical composition of any one of embodiments 129-131, wherein the monobasic sodium phosphate (monohydrate) is about 0.208 to 0.211 mg/mL.

Embodiment 133. The pharmaceutical composition of any one of embodiments 125, and 129-132, wherein 1 ml of the pharmaceutical composition comprises about 40 mg of the fitusiran, 7.672 mg of the sodium chloride, 0.933 mg of the dibasic sodium phosphate (heptahydrate), and 0.2095 mg of the monobasic sodium phosphate (monohydrate).

Embodiment 134. The pharmaceutical composition of embodiment 125, wherein 0.5 mL of the pharmaceutical composition comprises about 20 mg of fitusiran, 3.836 mg of sodium chloride, 0.467 mg of dibasic sodium phosphate, and 0.105 mg of monobasic sodium phosphate.

Embodiment 135. The pharmaceutical composition of any one of embodiments 125-134, wherein the pH of the composition is between about 5.0 to about 8.0.

Embodiment 136. The pharmaceutical composition of embodiment 135, wherein the pH of the composition is between about 6.0 to about 8.0.

Embodiment 137. The pharmaceutical composition of embodiment 136, wherein the pH of the composition is between about 6.5 to about 7.5.

Embodiment 138. The pharmaceutical composition of embodiment 137, wherein the pH of the composition is between about 6.8 to about 7.2.

Embodiment 139. The pharmaceutical composition of embodiment 138, wherein the pH of the composition is about 7.0.

Embodiment 140. The pharmaceutical composition of any one of embodiments 125-139, wherein the osmolality of the composition is between about 50 and about 400 mOsm/kg.

Embodiment 141. The pharmaceutical composition of embodiment 140, wherein the osmolality of the composition is between about 100 and about 400 mOsm/kg.

Embodiment 142. The pharmaceutical composition of embodiment 141, wherein the osmolality of the composition is between about 240 and about 390 mOsm/kg.

Embodiment 143. The pharmaceutical composition of embodiment 142, wherein the osmolality of the composition is about 300 mOsm/kg.

Embodiment 144. The pharmaceutical composition of any one of embodiments 125-143, wherein the composition is stable for up to about 36 months when stored at about 2° C. to about 8° C.

Embodiment 145. The pharmaceutical composition of any one of embodiments 125-144, wherein the composition is stable for up to about 36 months when stored at about 25° C. and 60% relative humidity (RH).

Embodiment 146. The pharmaceutical composition of any one of embodiments 125-145, wherein the composition is stable for up to about 6 months when stored at about 40° C. and 75% relative humidity (RH).

Embodiment 147. The pharmaceutical composition of any one of embodiments 125-146, wherein the composition comprises not less than (NLT) about 90.5 area % duplex and not more than (NMT) about 5 area % single strands as determined by purity non-denaturing IPRP-HPLC.

Embodiment 148. The pharmaceutical composition of any one of embodiments 125-147, wherein the composition comprises not less than (NLT) about 85.0 area % total single strands as determined by purity denaturing AX-HPLC.

Embodiment 149. The pharmaceutical composition of any one of embodiments 125-148, wherein the composition comprises not less than (NLT) about 80.0 area % total single strands as determined by purity denaturing IPRP-HPLC.

Embodiment 150. The pharmaceutical composition of any one of embodiments 125-149, wherein the composition comprises a density of about 1 mg/mL.

Embodiment 151. The pharmaceutical composition of any one of embodiments 125-150, wherein the composition comprises a density of about 1.02364 mg/mL.

Embodiment 152. The pharmaceutical composition of any one of embodiments 125-151, wherein the composition comprises a viscosity of about 1.4 pascal-seconds at about 20° C.

Embodiment 153. A pharmaceutical composition, comprising fitusiran at a concentration of about 40 mg/mL and phosphate buffered saline (PBS) at a concentration of about 5 mM, wherein the pH of the pharmaceutical composition is about 6.8 to about 7.2, and wherein the osmolality of the pharmaceutical composition is about 300 mOsm/kg.

Embodiment 154. A pharmaceutical composition comprising fitusiran and phosphate-buffered saline (PBS), wherein 1 ml of the pharmaceutical composition comprises about 40 mg of fitusiran, 7.672 mg of sodium chloride, 0.933 mg of dibasic sodium phosphate (heptahydrate), 0.2095 mg of monobasic sodium phosphate (monohydrate), and water for injection, wherein the pH of the pharmaceutical composition is about 6.8 to about 7.2.

Embodiment 155. A pharmaceutical composition comprising fitusiran and phosphate-buffered saline (PBS), wherein 0.5 mL of the pharmaceutical composition comprises about 20 mg of fitusiran, 3.836 mg of sodium chloride, 0.467 mg of dibasic sodium phosphate, 0.105 mg of monobasic sodium phosphate, and water for injection, wherein the pH of the pharmaceutical composition is about 6.8 to about 7.2.

Embodiment 156. The pharmaceutical composition of claim 154 or 155, wherein the pH of the pharmaceutical composition is about 7.0.

Embodiment 157. A method of treating hemophilia A or hemophilia B prophylactically in a patient in need thereof, comprising administering to the patient the pharmaceutical composition of any one of embodiments 125-156.

Embodiment 158. A use of the pharmaceutical composition of any one of embodiments 125-156 in the manufacture of a medicament for prophylactic treatment of hemophilia A or hemophilia B in a patient in need thereof.

Embodiment 159. The pharmaceutical composition of any one of embodiments 125-156 for use in the prophylactic treatment of hemophilia A or hemophilia B in a patient in need thereof.

Embodiment 160. The method of embodiment 157, the use of embodiment 158, or the pharmaceutical composition for use of embodiment 159, wherein the patient is a hemophilia A patient with inhibitors.

Embodiment 161. The method of embodiment 157, the use of embodiment 158, or the pharmaceutical composition for use of embodiment 159, wherein the patient is a hemophilia A patient without inhibitors.

Embodiment 162. The method of embodiment 157, the use of embodiment 158, or the pharmaceutical composition for use of embodiment 159, wherein the patient is a hemophilia B patient with inhibitors.

Embodiment 163. The method of embodiment 157, the use of embodiment 158, or the pharmaceutical composition for use of embodiment 159, wherein the patient is a hemophilia B patient without inhibitors.

Embodiment 164. A method of administering the pharmaceutical composition of any one of embodiments 125-156, comprising subcutaneous injection of the pharmaceutical composition with a prefilled syringe containing the pharmaceutical composition.

Embodiment 165. A method of administering the pharmaceutical composition of any one of embodiments 125-156, comprising subcutaneous injection of the pharmaceutical composition from a prefilled vial containing the pharmaceutical composition.

Embodiment 166. An article of manufacture comprising the pharmaceutical composition of any one of embodiments 125-156.

Embodiment 167. The article of manufacture of embodiment 166, wherein the article of manufacture is a syringe or a vial.

Embodiment 168. A vial comprising the pharmaceutical composition of any one of embodiments 125-156.

Embodiment 169. The vial of embodiment 168, wherein the vial comprises about 0.5 mL to about 2.0 ml of the pharmaceutical composition.

Embodiment 170. A syringe comprising the pharmaceutical composition of any one of embodiments 125-156.

Embodiment 171. The syringe of embodiment 170, wherein the syringe is a 1 ml syringe.

Embodiment 172. The syringe of embodiment 171, wherein the syringe comprises about 0.5 ml of the pharmaceutical composition.

Embodiment 173. The syringe of embodiment 172, wherein the syringe comprises 0.535 mL of the pharmaceutical composition.

Embodiment 174. The syringe of any one of embodiments 170-173, wherein the syringe is for injection of a 20 mg dose of fitusiran.

Embodiment 175. The syringe of embodiment 171, wherein the syringe comprises about 0.25 mL of the pharmaceutical composition.

Embodiment 176. The syringe of any one of embodiments 170-171 and 175, wherein the syringe is for injection of a 10 mg dose of fitusiran.

Embodiment 177. The syringe of any one of embodiments 170-176, wherein the syringe comprises a 29 G needle.

Embodiment 178. The syringe of any one of embodiments 170-176, wherein the syringe comprises a 30 G needle.

Embodiment 179. The syringe of any one of embodiments 170-178, wherein the syringe is a prefilled syringe.

Embodiment 180. The syringe of embodiment 179, wherein the prefilled syringe is a prefilled pen.

Embodiment 181. A kit comprising the article of manufacture of embodiment 166 or embodiment 167, the syringe of any one of embodiments 170-180, or the vial of embodiment 168 or embodiment 169, and a label or a package insert.

Embodiment 182. A method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising:
(1)
   (a) administering to the human patient fitusiran at a starting dose of 50 mg about once every 2 months; and
   (b) performing one of the following steps:
      (i) if an antithrombin (AT) level in the human patient is 15-35%, subcutaneously administering to the human patient fitusiran at a dose of 50 mg about once every 2 months,
      (ii) if an AT level in the human patient is >35% after 6 months, subcutaneously administering to the human patient fitusiran at a dose of 50 mg about once every month, or
      (iii) if an AT level in the human patient is <15%, subcutaneously administering to the human patient fitusiran at a dose of 20 mg about once every 2 months,
   wherein one or more measurements of an AT level in the human patient are or have been obtained before step (1)(a), after step (1)(a), and after any fitusiran dose modification;
(2)
   (a) administering to the human patient fitusiran at a dose of 20 mg about once every 2 months; and
   (b) performing one of the following steps:
      (i) if an AT level in the human patient is 15-35%, subcutaneously administering to the human patient fitusiran at a dose of 20 mg about once every 2 months,
      (ii) if an AT level in the human patient is >35% after 6 months, subcutaneously administering to the human patient fitusiran at a dose of 20 mg about once every month, or
      (iii) if an AT level in the human patient is <15%, subcutaneously administering to the human patient fitusiran at a dose of 10 mg about once every 2 months,
   wherein one or more measurements of an AT level in the human patient are or have been obtained before step (2)(a), after step (2)(a), and after any fitusiran dose modification; or
(3)
   (a) after administering to the human patient fitusiran at a dose of 10 mg about once every 2 months; and
   (b) performing one of the following steps:
      (i) if an AT level in the human patient is 15-35%, subcutaneously administering to the human patient fitusiran at a dose of 10 mg about once every 2 months,
      (ii) if an AT level in the human patient is >35% after 6 months, subcutaneously administering to the human patient fitusiran at a dose of 10 mg about once every month, or
      (iii) if an AT level in the human patient is <15%, discontinuing or pausing fitusiran treatment,
   wherein one or more measurements of an AT level in the human patient are or have been obtained before step (3)(a), after step (3)(a), and after any fitusiran dose modification.

Embodiment 183. A method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 50 mg about once every 2 months; and
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 50 mg about once every month, or
   (iii) if an AT level in the human patient is <15%, subcutaneously administering to the human patient fitusiran at a dose of 20 mg about once every 2 months,
wherein one or more measurements of an AT level in the human patient are or have been obtained before step (a), after step (a), and after any fitusiran dose modification.

Embodiment 184. A method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 20 mg about once every 2 months; and
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 20 mg about once every month, or
   (iii) if an AT level in the human patient is <15%, subcutaneously administering to the human patient fitusiran at a dose of 10 mg about once every 2 months, wherein one or more measurements of an AT level in the human patient are or have been obtained before step (a), after step (a), and after any fitusiran dose modification.

Embodiment 185. A method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 10 mg about once every 2 months;
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 10 mg about once every month, or
   (iii) if an AT level in the human patient is <15%, discontinuing or pausing fitusiran treatment,
wherein one or more measurements of an AT level in the human patient are or have been obtained before step (a), after step (a), and after any fitusiran dose modification.

Embodiment 186. A method of reducing a risk of gallbladder disease in a human patient with hemophilia A or B with or without factor VIII or factor IX inhibitors who receives fitusiran for routine prophylaxis to prevent or reduce the frequency of bleeding episodes, comprising:
(1)
   (a) administering to the human patient fitusiran at a starting dose of 50 mg about once every 2 months; and
   (b) performing one of the following steps:
      (i) if an antithrombin (AT) level in the human patient is 15-35%, subcutaneously administering to the human patient fitusiran at a dose of 50 mg about once every 2 months,
      (ii) if an AT level in the human patient is >35% after 6 months, subcutaneously administering to the human patient fitusiran at a dose of 50 mg about once every month, or
      (iii) if an AT level in the human patient is <15%, subcutaneously administering to the human patient fitusiran at a dose of 20 mg about once every 2 months,
   wherein one or more measurements of an AT level in the human patient are or have been obtained before step (1)(a), after step (1)(a), and after any fitusiran dose modification;
(2)
   (a) administering to the human patient fitusiran at a dose of 20 mg about once every 2 months; and
   (b) performing one of the following steps:
      (i) if an AT level in the human patient is 15-35%, subcutaneously administering to the human patient fitusiran at a dose of 20 mg about once every 2 months,
      (ii) if an AT level in the human patient is >35% after 6 months, subcutaneously administering to the human patient fitusiran at a dose of 20 mg about once every month, or
      (iii) if an AT level in the human patient is <15%, subcutaneously administering to the human patient fitusiran at a dose of 10 mg about once every 2 months, wherein one or more measurements of an AT level in the human patient are or have been obtained before step (2)(a), after step (2)(a), and after any fitusiran dose modification; or
(3)
   (a) after administering to the human patient fitusiran at a dose of 10 mg about once every 2 months; and
   (b) performing one of the following steps:
      (i) if an AT level in the human patient is 15-35%, subcutaneously administering to the human patient fitusiran at a dose of 10 mg about once every 2 months,
      (ii) if an AT level in the human patient is >35% after 6 months, subcutaneously administering to the human patient fitusiran at a dose of 10 mg about once every month, or
      (iii) if an AT level in the human patient is <15%, discontinuing or pausing fitusiran treatment,
   wherein one or more measurements of an AT level in the human patient are or have been obtained before step (3)(a), after step (3)(a), and after any fitusiran dose modification.

Embodiment 187. A method of reducing a risk of gallbladder disease in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 50 mg about once every 2 months; and
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 50 mg about once every month, or
   (iii) if an AT level in the human patient is <15%, subcutaneously administering to the human patient fitusiran at a dose of 20 mg about once every 2 months,
wherein one or more measurements of an AT level in the human patient are or have been obtained before step (a), after step (a), and after any fitusiran dose modification.

Embodiment 188. A method of reducing a risk of gallbladder disease in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 20 mg about once every 2 months; and
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 20 mg about once every month, or
   (iii) if an AT level in the human patient is <15%, subcutaneously administering to the human patient fitusiran at a dose of 10 mg about once every 2 months,
wherein one or more measurements of an AT level in the human patient are or have been obtained before step (a), after step (a), and after any fitusiran dose modification.

Embodiment 189. A method of reducing a risk of gallbladder disease in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 10 mg about once every 2 months;
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 10 mg about once every month, or
   (iii) if an AT level in the human patient is <15%, discontinuing or pausing fitusiran treatment,
wherein one or more measurements of an AT level in the human patient are or have been obtained before step (a), after step (a), and after any fitusiran dose modification.

Embodiment 190. A method of reducing thrombotic risk in a human patient with hemophilia A or B with or without factor VIII or factor IX inhibitors who receives fitusiran for routine prophylaxis to prevent or reduce the frequency of bleeding episodes, comprising:
(1)
   (a) administering to the human patient fitusiran at a starting dose of 50 mg about once every 2 months; and
   (b) performing one of the following steps:
      (i) if an antithrombin (AT) level in the human patient is 15-35%, subcutaneously administering to the human patient fitusiran at a dose of 50 mg about once every 2 months,
      (ii) if an AT level in the human patient is >35% after 6 months, subcutaneously administering to the human patient fitusiran at a dose of 50 mg about once every month, or
      (iii) if an AT level in the human patient is <15%, subcutaneously administering to the human patient fitusiran at a dose of 20 mg about once every 2 months,
   wherein one or more measurements of an AT level in the human patient are or have been obtained before step (1)(a), after step (1)(a), and after any fitusiran dose modification;
(2)
   (a) administering to the human patient fitusiran at a dose of 20 mg about once every 2 months; and
   (b) performing one of the following steps:
      (i) if an AT level in the human patient is 15-35%, subcutaneously administering to the human patient fitusiran at a dose of 20 mg about once every 2 months,
      (ii) if an AT level in the human patient is >35% after 6 months, subcutaneously administering to the human patient fitusiran at a dose of 20 mg about once every month, or
      (iii) if an AT level in the human patient is <15%, subcutaneously administering to the human patient fitusiran at a dose of 10 mg about once every 2 months,
   wherein one or more measurements of an AT level in the human patient are or have been obtained before step (2)(a), after step (2)(a), and after any fitusiran dose modification; or
(3)
   (a) after administering to the human patient fitusiran at a dose of 10 mg about once every 2 months; and
   (b) performing one of the following steps:
      (i) if an AT level in the human patient is 15-35%, subcutaneously administering to the human patient fitusiran at a dose of 10 mg about once every 2 months,
      (ii) if an AT level in the human patient is >35% after 6 months, subcutaneously administering to the human patient fitusiran at a dose of 10 mg about once every month, or
      (iii) if an AT level in the human patient is <15%, discontinuing or pausing fitusiran treatment,
   wherein one or more measurements of an AT level in the human patient are or have been obtained before step (3)(a), after step (3)(a), and after any fitusiran dose modification.

Embodiment 191. A method of reducing thrombotic risk in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 50 mg about once every 2 months; and
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 50 mg about once every month, or
   (iii) if an AT level in the human patient is <15%, subcutaneously administering to the human patient fitusiran at a dose of 20 mg about once every 2 months,
wherein one or more measurements of an AT level in the human patient are or have been obtained before step (a), after step (a), and after any fitusiran dose modification.

Embodiment 192. A method of reducing thrombotic risk in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 20 mg about once every 2 months; and
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 20 mg about once every month, or
   (iii) if an AT level in the human patient is <15%, subcutaneously administering to the human patient fitusiran at a dose of 10 mg about once every 2 months, wherein one or more measurements of an AT level in the human patient are or have been obtained before step (a), after step (a), and after any fitusiran dose modification.

Embodiment 193. A method of reducing thrombotic risk in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 10 mg about once every 2 months;
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 10 mg about once every month, or
   (iii) if an AT level in the human patient is <15%, discontinuing or pausing fitusiran treatment,
wherein one or more measurements of an AT level in the human patient are or have been obtained before step (a), after step (a), and after any fitusiran dose modification.

Embodiment 194. A method of routine prophylaxis to prevent or reduce the frequency of bleeding episodes in a human patient with hemophilia A or B with or without factor VIII or factor IX inhibitors, comprising:
(1)
   (a) administering to the human patient fitusiran at a starting dose of 50 mg about once every 2 months; and
   (b) performing one of the following steps:
      (i) if an antithrombin (AT) level in the human patient is 15-35%, subcutaneously administering to the human patient fitusiran at a dose of 50 mg about once every 2 months,
      (ii) if an AT level in the human patient is >35% after 6 months, subcutaneously administering to the human patient fitusiran at a dose of 50 mg about once every month, or
      (iii) if an AT level in the human patient is <15%, subcutaneously administering to the human patient fitusiran at a dose of 20 mg about once every 2 months,
   wherein one or more measurements of an AT level in the human patient are or have been obtained before step (1)(a), after step (1)(a), and after any fitusiran dose modification;
(2)
   (a) administering to the human patient fitusiran at a dose of 20 mg about once every 2 months; and
   (b) performing one of the following steps:
      (i) if an AT level in the human patient is 15-35%, subcutaneously administering to the human patient fitusiran at a dose of 20 mg about once every 2 months,
      (ii) if an AT level in the human patient is >35% after 6 months, subcutaneously administering to the human patient fitusiran at a dose of 20 mg about once every month, or
      (iii) if an AT level in the human patient is <15%, subcutaneously administering to the human patient fitusiran at a dose of 10 mg about once every 2 months,
   wherein one or more measurements of an AT level in the human patient are or have been obtained before step (2)(a), after step (2)(a), and after any fitusiran dose modification; or
(3)
   (a) after administering to the human patient fitusiran at a dose of 10 mg about once every 2 months; and
   (b) performing one of the following steps:
      (i) if an AT level in the human patient is 15-35%, subcutaneously administering to the human patient fitusiran at a dose of 10 mg about once every 2 months,
      (ii) if an AT level in the human patient is >35% after 6 months, subcutaneously administering to the human patient fitusiran at a dose of 10 mg about once every month, or
      (iii) if an AT level in the human patient is <15%, discontinuing or pausing fitusiran treatment,
   wherein one or more measurements of an AT level in the human patient are or have been obtained before step (3)(a), after step (3)(a), and after any fitusiran dose modification.

Embodiment 195. A method of routine prophylaxis to prevent or reduce the frequency of bleeding episodes in a human patient with hemophilia A or B with or without factor VIII or factor IX inhibitors, comprising:
(a) subcutaneously administering to the human patient in need thereof fitusiran at a dose of 50 mg about once every 2 months; and
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 50 mg about once every month, or
   (iii) if an AT level in the human patient is <15%, subcutaneously administering to the human patient fitusiran at a dose of 20 mg about once every 2 months,
wherein one or more measurements of an AT level in the human patient are or have been obtained before step (a), after step (a), and after any fitusiran dose modification.

Embodiment 196. A method of routine prophylaxis to prevent or reduce the frequency of bleeding episodes in a human patient with hemophilia A or B with or without factor VIII or factor IX inhibitors, comprising:
(a) subcutaneously administering to the human patient in need thereof fitusiran at a dose of 20 mg about once every 2 months; and
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 20 mg about once every month, or
   (iii) if an AT level in the human patient is <15%, subcutaneously administering to the human patient fitusiran at a dose of 10 mg about once every 2 months,
wherein one or more measurements of an AT level in the human patient are or have been obtained before step (a), after step (a), and after any fitusiran dose modification.

Embodiment 197. A method of routine prophylaxis to prevent or reduce the frequency of bleeding episodes in a human patient with hemophilia A or B with or without factor VIII or factor IX inhibitors, comprising:
(a) subcutaneously administering to the human patient in need thereof fitusiran at a dose of 10 mg about once every 2 months;
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 10 mg about once every month, or
   (iii) if an AT level in the human patient is <15%, discontinuing or pausing fitusiran treatment,
wherein one or more measurements of an AT level in the human patient are or have been obtained before step (a), after step (a), and after any fitusiran dose modification.

Embodiment 198. A method of reducing annualized bleeding rate in a human patient with hemophilia A or B with or without factor VIII or factor IX inhibitors who receives fitusiran for routine prophylaxis to prevent or reduce the frequency of bleeding episodes, comprising:
(1)
   (a) administering to the human patient fitusiran at a starting dose of 50 mg about once every 2 months; and
   (b) performing one of the following steps:
      (i) if an antithrombin (AT) level in the human patient is 15-35%, subcutaneously administering to the human patient fitusiran at a dose of 50 mg about once every 2 months,
      (ii) if an AT level in the human patient is >35% after 6 months, subcutaneously administering to the human patient fitusiran at a dose of 50 mg about once every month, or
      (iii) if an AT level in the human patient is <15%, subcutaneously administering to the human patient fitusiran at a dose of 20 mg about once every 2 months,
   wherein one or more measurements of an AT level in the human patient are or have been obtained before step (1)(a), after step (1)(a), and after any fitusiran dose modification;
(2)
   (a) administering to the human patient fitusiran at a dose of 20 mg about once every 2 months; and
   (b) performing one of the following steps:
      (i) if an AT level in the human patient is 15-35%, subcutaneously administering to the human patient fitusiran at a dose of 20 mg about once every 2 months,
      (ii) if an AT level in the human patient is >35% after 6 months, subcutaneously administering to the human patient fitusiran at a dose of 20 mg about once every month, or
      (iii) if an AT level in the human patient is <15%, subcutaneously administering to the human patient fitusiran at a dose of 10 mg about once every 2 months,
   wherein one or more measurements of an AT level in the human patient are or have been obtained before step (2)(a), after step (2)(a), and after any fitusiran dose modification; or
(3)
   (a) after administering to the human patient fitusiran at a dose of 10 mg about once every 2 months; and
   (b) performing one of the following steps:
      (i) if an AT level in the human patient is 15-35%, subcutaneously administering to the human patient fitusiran at a dose of 10 mg about once every 2 months,
      (ii) if an AT level in the human patient is >35% after 6 months, subcutaneously administering to the human patient fitusiran at a dose of 10 mg about once every month, or
      (iii) if an AT level in the human patient is <15%, discontinuing or pausing fitusiran treatment,
   wherein one or more measurements of an AT level in the human patient are or have been obtained before step (3)(a), after step (3)(a), and after any fitusiran dose modification.

Embodiment 199. A method of reducing annualized bleeding rate in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 50 mg about once every 2 months; and
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 50 mg about once every month, or
   (iii) if an AT level in the human patient is <15%, subcutaneously administering to the human patient fitusiran at a dose of 20 mg about once every 2 months,
wherein one or more measurements of an AT level in the human patient are or have been obtained before step (a), after step (a), and after any fitusiran dose modification.

Embodiment 200. A method of reducing annualized bleeding rate in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 20 mg about once every 2 months; and
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 20 mg about once every month, or
   (iii) if an AT level in the human patient is <15%, subcutaneously administering to the human patient fitusiran at a dose of 10 mg about once every 2 months,
wherein one or more measurements of an AT level in the human patient are or have been obtained before step (a), after step (a), and after any fitusiran dose modification.

Embodiment 201. A method of reducing annualized bleeding rate in a human patient with hemophilia A or B with or without factor VIII or factor IX inhibitors who receives fitusiran for routine prophylaxis to prevent or reduce the frequency of bleeding episodes, comprising:
(a) subcutaneously administering to the human patient in need thereof fitusiran at a dose of 10 mg about once every 2 months;
(b) performing one of the following steps:
   (i) if an antithrombin (AT) level in the human patient is 15-35%, repeating step (a),
   (ii) if an AT level in the human patient is >35% after 6 months, subcutaneously administering to the human patient fitusiran at a dose of 10 mg about once every month, or
   (iii) if an AT level in the human patient is <15%, discontinuing or pausing fitusiran treatment,
   wherein one or more measurements of an AT level in the human patient are or have been obtained before step (a), after step (a), and after any fitusiran dose modification.

Embodiment 202. The method of any one of embodiments 1-103, 123-124, 157, 160-163, 164-165, and 182-201, the fitusiran for use of embodiment 104, the use of embodiment 105 or 158, the pharmaceutical composition of any one of embodiments 106, 125-156, and 159, the article of manufacture of any one of embodiments 107-116 and 166-167, the package of any one of embodiments 117-122, the vial of embodiment 168 or 169, the syringe of any one of embodiments 170-180, or the kit of embodiment 181, wherein fitusiran is in a pharmaceutically acceptable salt form.

Embodiment 203. The method of any one of embodiments 1-103, 123-124, 157, 160-163, and 164-165, and 182-201, the fitusiran for use of embodiment 104, the use of embodiment 105 or 158, the pharmaceutical composition of any one of embodiments 106, 125-156, and 159, the article of manufacture of any one of embodiments 107-116 and 166-167, the package of any one of embodiments 117-122, the vial of embodiment 168 or 169, the syringe of any one of embodiments 170-180, or the kit of embodiment 181, wherein fitusiran is in a sodium salt form.

Further exemplary embodiments of the methods and compositions described herein include:

Embodiment 1A. A method of routine prophylaxis to prevent or reduce the frequency of bleeding episodes in a human patient having hemophilia A or B with or without factor VIII or factor IX inhibitors, comprising: (a) subcutaneously administering to the human patient in need thereof fitusiran at a starting dose of 50 mg about once every 2 months; (b) obtaining a measurement of an antithrombin (AT) level in the human patient; (c) performing one of the following steps: (i) if the AT level is 15-35%, repeating step (a), (ii) if the AT level is >35% after 6 months, subcutaneously administering to the human patient fitusiran at a dose of 50 mg about once every month, or (iii) if the AT level is <15%, subcutaneously administering to the human patient fitusiran at a dose of 20 mg about once every 2 months; (d) after administering to the human patient fitusiran at a dose of 20 mg about once every 2 months, obtaining a measurement of an antithrombin (AT) level in the human patient; (e) performing one of the following steps: (i) if the AT level is 15-35%, subcutaneously administering to the human patient fitusiran at a dose of 20 mg about once every 2 months, (ii) if the AT level is >35% after 6 months, subcutaneously administering to the human patient fitusiran at a dose of 20 mg about once every month, or (iii) if the AT level is <15%, subcutaneously administering to the human patient fitusiran at a dose of 10 mg about once every 2 months; (f) after administering to the human patient fitusiran at a dose of 10 mg about once every 2 months, obtaining a measurement of an antithrombin (AT) level in the human patient; and (g) performing one of the following steps: (i) if the AT level is 15-35%, subcutaneously administering to the human patient fitusiran at a dose of 10 mg about once every 2 months, (ii) if the AT level is >35% after 6 months, subcutaneously administering to the human patient fitusiran at a dose of 10 mg about once every month, or (iii) if the AT level is <15%, discontinuing or pausing fitusiran treatment.

Embodiment 2A. A method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising: (a) obtaining a measurement of an antithrombin (AT) level in the human patient, wherein the human patient has previously received fitusiran at a starting dose of 50 mg about once every 2 months; (b) performing one of the following steps: (i) if the AT level is 15-35%, subcutaneously administering to the human patient fitusiran at a dose of 50 mg about once every 2 months, (ii) if the AT level is >35% after 6 months, subcutaneously administering to the human patient fitusiran at a dose of 50 mg about once every month, or (iii) if the AT level is <15%, subcutaneously administering to the human patient fitusiran at a dose of 20 mg about once every 2 months; (c) after administering to the human patient fitusiran at a dose of 20 mg about once every 2 months, obtaining a measurement of an antithrombin (AT) level in the human patient; (d) performing one of the following steps: (i) if the AT level is 15-35%, subcutaneously administering to the human patient fitusiran at a dose of 20 mg about once every 2 months, (ii) if the AT level is >35% after 6 months, subcutaneously administering to the human patient fitusiran at a dose of 20 mg about once every month, or (iii) if the AT level is <15%, subcutaneously administering to the human patient fitusiran at a dose of 10 mg about once every 2 months; (e) after administering to the human patient fitusiran at a dose of 10 mg about once every 2 months, obtaining a measurement of an antithrombin (AT) level in the human patient; and (f) performing one of the following steps: (i) if the AT level is 15-35%, subcutaneously administering to the human patient fitusiran at a dose of 10 mg about once every 2 months, (ii) if the AT level is >35% after 6 months, subcutaneously administering to the human patient fitusiran at a dose of 10 mg about once every month, or (iii) if the AT level is <15%, discontinuing or pausing fitusiran treatment.

Embodiment 3A. The method of embodiment 2A, wherein reducing the risk of hepatotoxicity comprises reducing the occurrence in the human patient of alanine transaminase (ALT) elevation more than three times the upper limit of normal (ULN), aspartate aminotransferase (AST) elevation more than three times the ULN, severe liver toxicity, liver failure, and/or jaundice.

Embodiment 4A. A method of reducing a risk of gallbladder disease in a human patient with hemophilia A or B with or without factor VIII or factor IX inhibitors who receives fitusiran for routine prophylaxis to prevent or reduce the frequency of bleeding episodes, comprising: (a) obtaining a measurement of an antithrombin (AT) level in the human patient, wherein the human patient has previously received fitusiran at a starting dose of 50 mg about once every 2 months; (b) performing one of the following steps: (i) if the AT level is 15-35%, subcutaneously administering to the human patient fitusiran at a dose of 50 mg about once every 2 months, (ii) if the AT level is >35% after 6 months, subcutaneously administering to the human patient fitusiran at a dose of 50 mg about once every month, or (iii) if the AT level is <15%, subcutaneously administering to the human patient fitusiran at a dose of 20 mg about once every 2 months; (c) after administering to the human patient fitusiran at a dose of 20 mg about once every 2 months, obtaining a measurement of an antithrombin (AT) level in the human patient; (d) performing one of the following steps: (i) if the AT level is 15-35%, subcutaneously administering to the human patient fitusiran at a dose of 20 mg about once every 2 months, (ii) if the AT level is >35% after 6 months, subcutaneously administering to the human patient fitusiran at a dose of 20 mg about once every month, or (iii) if the AT level is <15%, subcutaneously administering to the human patient fitusiran at a dose of 10 mg about once every 2 months; (e) after administering to the human patient fitusiran at a dose of 10 mg about once every 2 months, obtaining a measurement of an antithrombin (AT) level in the human patient; and (f) performing one of the following steps: (i) if the AT level is 15-35%, subcutaneously administering to the human patient fitusiran at a dose of 10 mg about once every 2 months, (ii) if the AT level is >35% after 6 months, subcutaneously administering to the human patient fitusiran at a dose of 10 mg about once every month, or (iii) if the AT level is <15%, discontinuing or pausing fitusiran treatment.

Embodiment 5A. The method of embodiment 4A, wherein reducing the risk of gallbladder disease comprises reducing the occurrence in the human patient of cholecystitis, cholelithiasis, or a need for cholecystectomy.

Embodiment 6A. A method of reducing thrombotic risk in a human patient with hemophilia A or B with or without factor VIII or factor IX inhibitors who receives fitusiran for routine prophylaxis to prevent or reduce the frequency of bleeding episodes, comprising: (a) obtaining a measurement of an antithrombin (AT) level in the human patient, wherein the human patient has previously received fitusiran at a starting dose of 50 mg about once every 2 months; (b) performing one of the following steps: (i) if the AT level is 15-35%, subcutaneously administering to the human patient fitusiran at a dose of 50 mg about once every 2 months, (ii) if the AT level is >35% after 6 months, subcutaneously administering to the human patient fitusiran at a dose of 50 mg about once every month, or (iii) if the AT level is <15%, subcutaneously administering to the human patient fitusiran at a dose of 20 mg about once every 2 months; (c) after administering to the human patient fitusiran at a dose of 20 mg about once every 2 months, obtaining a measurement of an antithrombin (AT) level in the human patient; (d) performing one of the following steps: (i) if the AT level is 15-35%, subcutaneously administering to the human patient fitusiran at a dose of 20 mg about once every 2 months, (ii) if the AT level is >35% after 6 months, subcutaneously administering to the human patient fitusiran at a dose of 20 mg about once every month, or (iii) if the AT level is <15%, subcutaneously administering to the human patient fitusiran at a dose of 10 mg about once every 2 months; (e) after administering to the human patient fitusiran at a dose of 10 mg about once every 2 months, obtaining a measurement of an antithrombin (AT) level in the human patient; and (f) performing one of the following steps: (i) if the AT level is 15-35%, subcutaneously administering to the human patient fitusiran at a dose of 10 mg about once every 2 months, (ii) if the AT level is >35% after 6 months, subcutaneously administering to the human patient fitusiran at a dose of 10 mg about once every month, or (iii) if the AT level is <15%, discontinuing or pausing fitusiran treatment.

Embodiment 7A. The method of embodiment 6A, wherein reducing thrombotic risk comprises reducing the occurrence in the human patient of cerebrovascular accident, cerebral artery embolism, spinal vascular disorder, atrial thrombosis, cerebral venous sinus thrombosis, thrombosis, thrombosis on papillae of an eye, deep venous thrombosis of an arm, subclavian vein thrombosis, superficial venous thrombosis of an arm, cerebral infarction, embolic stroke, or postoperative venous thrombosis.

Embodiment 8A. A method of monitoring an antithrombin level in a human patient with hemophilia A or B with or without factor VIII or factor IX inhibitors who receives fitusiran for routine prophylaxis to prevent or reduce the frequency of bleeding episodes, comprising: (a) obtaining a measurement of an antithrombin (AT) level in the human patient, wherein the human patient has previously received fitusiran at a starting dose of 50 mg about once every 2 months; (b) performing one of the following steps: (i) if the AT level is 15-35%, subcutaneously administering to the human patient fitusiran at a dose of 50 mg about once every 2 months, (ii) if the AT level is >35% after 6 months, subcutaneously administering to the human patient fitusiran at a dose of 50 mg about once every month, or (iii) if the AT level is <15%, subcutaneously administering to the human patient fitusiran at a dose of 20 mg about once every 2 months; (c) after administering to the human patient fitusiran at a dose of 20 mg about once every 2 months, obtaining a measurement of an antithrombin (AT) level in the human patient; (d) performing one of the following steps: (i) if the AT level is 15-35%, subcutaneously administering to the human patient fitusiran at a dose of 20 mg about once every 2 months, (ii) if the AT level is >35% after 6 months, subcutaneously administering to the human patient fitusiran at a dose of 20 mg about once every month, or (iii) if the AT level is <15%, subcutaneously administering to the human patient fitusiran at a dose of 10 mg about once every 2 months; (e) after administering to the human patient fitusiran at a dose of 10 mg about once every 2 months, obtaining a measurement of an antithrombin (AT) level in the human patient; and (f) performing one of the following steps: (i) if the AT level is 15-35%, subcutaneously administering to the human patient fitusiran at a dose of 10 mg about once every 2 months, (ii) if the AT level is >35% after 6 months, subcutaneously administering to the human patient fitusiran at a dose of 10 mg about once every month, or (iii) if the AT level is <15%, discontinuing or pausing fitusiran treatment.

Embodiment 9A. The method of embodiment 8A, wherein the antithrombin level is monitored using a kinetic or chromogenic assay, wherein the chromogenic assay quantifies functionally active AT in human citrated plasma based on the inhibition of an excess of factor Xa by AT.

Embodiment 10A. A method of reducing annualized bleeding rate in a human patient with hemophilia A or B with or without factor VIII or factor IX inhibitors who receives fitusiran for routine prophylaxis to prevent or reduce the frequency of bleeding episodes, comprising: (a) obtaining a measurement of an antithrombin (AT) level in the human patient, wherein the human patient has previously received fitusiran at a starting dose of 50 mg about once every 2 months; (b) performing one of the following steps: (i) if the AT level is 15-35%, subcutaneously administering to the human patient fitusiran at a dose of 50 mg about once every 2 months, (ii) if the AT level is >35% after 6 months, subcutaneously administering to the human patient fitusiran at a dose of 50 mg about once every month, or (iii) if the AT level is <15%, subcutaneously administering to the human patient fitusiran at a dose of 20 mg about once every 2 months; (c) after administering to the human patient fitusiran at a dose of 20 mg about once every 2 months, obtaining a measurement of an antithrombin (AT) level in the human patient; (d) performing one of the following steps: (i) if the AT level is 15-35%, subcutaneously administering to the human patient fitusiran at a dose of 20 mg about once every 2 months, (ii) if the AT level is >35% after 6 months, subcutaneously administering to the human patient fitusiran at a dose of 20 mg about once every month, or (iii) if the AT level is <15%, subcutaneously administering to the human patient fitusiran at a dose of 10 mg about once every 2 months; (e) after administering to the human patient fitusiran at a dose of 10 mg about once every 2 months, obtaining a measurement of an antithrombin (AT) level in the human patient; and (f) performing one of the following steps: (i) if the AT level is 15-35%, subcutaneously administering to the human patient fitusiran at a dose of 10 mg about once every 2 months, (ii) if the AT level is >35% after 6 months, subcutaneously administering to the human patient fitusiran at a dose of 10 mg about once every month, or (iii) if the AT level is <15%, discontinuing or pausing fitusiran treatment.

Embodiment 11A. The method of any one of embodiments 1A-10A, further comprising administering an effective amount of a replacement factor or bypassing agent (BPA) to treat a bleeding episode that occurs eight or more days after the first fitusiran dose, wherein the effective amount of the replacement factor or BPA is reduced as compared to the recommended effective amount of the replacement factor or BPA for human patients who are not on fitusiran therapy.

Embodiment 12A. The method of embodiment 11A, wherein the effective amount of the replacement factor or BPA is reduced to one half of a weight-based dose of the recommended effective amount of the replacement factor or BPA for human patients who are not on fitusiran therapy, administered at double the dosing frequency recommended for human patients who are not on fitusiran therapy.

Embodiment 13A. The method of any one of embodiments 1A-12A, wherein: (a) the human patient is a hemophilia A patient without factor VIII inhibitors, and wherein the replacement factor is factor VIII (FVIII) and a single dose of FVIII is no more than 20 IU/kg; or (b) the human patient is a hemophilia B patient without factor IX inhibitors, and wherein the replacement factor is factor IX (FIX) and a single dose of FIX is no more than 30 IU/kg.

Embodiment 14A. The method of any one of embodiments 1A-13A, wherein the human patient is a hemophilia A patient with factor VIII inhibitors or a hemophilia B patient with factor IX inhibitors, wherein the BPA is activated prothrombin complex concentrate (aPCC) and a single dose of aPCC is no more than 50 U/kg, or wherein the BPA is recombinant factor VIIa (rFVIIa) and a single dose of rFVIIa is no more than 45 g/kg.

Embodiment 15A. The method of any one of embodiments 1A-10A, further comprising administering an effective amount of a replacement factor or bypassing agent (BPA) to treat a bleeding episode that occurs seven or fewer days after the first fitusiran dose, wherein the effective amount of the replacement factor or BPA is administered according to the human patient's prior dosing regimen of replacement factor or BPA.

Embodiment 16A. The method of any one of embodiments 1A-15A, wherein the human patient has been on prophylactic treatment with a replacement factor or a bypassing agent (BPA), and wherein the method comprises terminating the prophylactic replacement factor or BPA treatment in the human patient within about two weeks or about 14 days or about one week or about seven days of the first dose of fitusiran.

Embodiment 17A. The method of any one of embodiments 1A-16A, comprising obtaining a measurement of AT level in the human patient about every four weeks, about every eight weeks, about every month, about every two months, about every four months, about every six months, or about every 12 months, and/or at four weeks or one month, twelve weeks or three months, 20 weeks or five months, and 24 weeks or six months following administration of the starting dose or following administration of a modified dose as compared to the prior dose.

Embodiment 18A. The method of any one of embodiments 1A-17A, wherein the obtaining a measurement of the AT level in the human patient comprises use of a kinetic or chromogenic assay.

Embodiment 19A. The method of any one of embodiments 1A-18A, further comprising subcutaneously administering fitusiran to the human patient at a dose amount and a dosing frequency sufficient to maintain the AT level in the human patient at 15-35%.

Embodiment 20A. The method of embodiment 19A, further comprising subcutaneously administering fitusiran at: 10 mg QM or Q4W, 10 mg Q2M or Q8W, 20 mg QM or Q4W, 20 mg Q2M or Q8W, 50 mg QM or Q4W, or 50 mg Q2M or Q8W.

Embodiment 21A. The method of any one of embodiments 1A-20A, wherein the human patient does not have (i) clinically significant liver disease, (ii) ALT >1.5× upper limit of normal reference range (ULN), (iii) AST >1.5× upper limit of normal reference range (ULN), (iv) hepatitis C, (v) hepatitis A, (vi) hepatitis E, (vii) hepatitis B, (viii) acute and recurrent gallbladder disease, (ix) symptomatic gallbladder disease, (x) established thrombophilic conditions, (xi) a prior history of thrombosis, (xii) an indwelling venous catheter, (xiii) persistent AT activity <15%, or (xiv) known hepatic impairment.

Embodiment 22A. A pharmaceutical composition, comprising fitusiran at a concentration of about 35 mg/ml to about 45 mg/mL and phosphate buffered saline (PBS) at a concentration of about 1 mM to about 10 mM, wherein the pH and the osmolality of the pharmaceutical composition are suitable for subcutaneous administration to a subject, and wherein the composition comprises about 7.67 to about 7.69 mg/mL sodium chloride, about 0.932 to about 0.934 mg/mL dibasic sodium phosphate (heptahydrate), and about 0.208 to about 0.211 mg/mL monobasic sodium phosphate (monohydrate), wherein the pH of the composition is between about 5.0 to about 8.0.

Embodiment 23A. A pharmaceutical composition comprising fitusiran, wherein: (a) 0.5 mL of the pharmaceutical composition comprises about 50 mg of fitusiran (equivalent to about 53.0 mg fitusiran sodium), about 0.585 mg dibasic sodium phosphate, about 0.044 mg monobasic sodium phosphate, about 2.455 mg sodium chloride, and water for injection, wherein the pH of the pharmaceutical composition is about 7.0; (b) 0.5 mL of the pharmaceutical composition comprises about 20 mg of fitusiran (equivalent to about 21.2 mg fitusiran sodium), about 0.467 mg dibasic sodium phosphate, about 0.105 mg monobasic sodium phosphate, about 3.836 mg sodium chloride, and water for injection, wherein the pH of the pharmaceutical composition is about 7.0; or (c) 0.2 mL of the pharmaceutical composition comprises about 20 mg of fitusiran (equivalent to about 21.2 mg fitusiran sodium), about 0.234 mg dibasic sodium phosphate, about 0.018 mg monobasic sodium phosphate, about 0.982 mg sodium chloride, and water for injection.

Embodiment 24A. A method of treating hemophilia A or hemophilia B prophylactically in a patient in need thereof, comprising administering to the patient the pharmaceutical composition of embodiment 22A or 23A, wherein administering the pharmaceutical composition comprises subcutaneous injection of the pharmaceutical composition from a prefilled syringe or a prefilled vial containing the pharmaceutical composition.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of hematology, medicine, medicinal and pharmaceutical chemistry, and cell biology described herein are those well-known and commonly used in the art. Further, unless otherwise required by context, singular terms shall include pluralities and plural terms shall include the singular. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Safety and Efficacy of an AT-Based Dose Regimen for Fitusiran

### Highlights of Prescribing Information

These highlights do not include all the information needed to use fitusiran safely and effectively. See full prescribing information for fitusiran.

### Warning: Thrombotic Events and Acute and Recurrent Gallbladder Disease

See full prescribing information for complete boxed warning.

### Thrombotic Events

Serious thrombotic events have occurred in fitusiran-treated patients with risk factors including persistent antithrombin (AT) activity less than 15%, fitusiran 80 mg once monthly dosing, an indwelling venous catheter, and the post-operative setting when bleed management guidelines were not followed.

Interrupt fitusiran in patients with a thrombotic event and manage as clinically indicated. (5.1)

### Acute and Recurrent Gallbladder Disease

Gallbladder disease has occurred in fitusiran-treated patients, some requiring cholecystectomy or developing complications (e.g., pancreatitis).

Monitor for signs and symptoms of gallbladder disease. Consider interruption or discontinuation of fitusiran if gallbladder disease occurs.

Consider alternative treatment for hemophilia if history of symptomatic gallbladder disease. (5.2)

### Indications and Usage

Fitusiran is an antithrombin-directed small interfering ribonucleic acid indicated for routine prophylaxis to prevent or reduce the frequency of bleeding episodes in adult and pediatric patients aged 12 years and older with hemophilia A or B with or without factor VIII or IX inhibitors. (1)

### Dosage and Administration

For subcutaneous use only.

Starting dose: 50 mg once every 2 months (2.1).

Monitor AT activity using an FDA-cleared test.

Maintain AT activity between 15-35% by adjusting the dose and/or frequency of administration (2.2).

See Full Prescribing Information for important preparation and administration instructions (2.4).

### Dosage Forms and Strengths

### Injection:

50 mg/0.5 mL (100 mg/mL) in a single-dose prefilled pen
20 mg/0.5 mL (40 mg/mL) in a single-dose prefilled pen
20 mg/0.2 mL (100 mg/mL) in a single-dose vial (3)

### Contraindications

None (4)

### Warnings and Precautions

Hepatotoxicity: Obtain liver tests at baseline and then monthly for at least 6 months after initiating fitusiran and after dose increases, and periodically thereafter. Liver test elevations may require fitusiran interruption or discontinuation (5.3).

### Adverse Reactions

Common adverse reactions (incidence >10%) are viral infection, nasopharyngitis, and bacterial infection (6.1).

### Use in Specific Populations

Avoid use in patients with established hepatic impairment (Child-Pugh Class A, B and C) (8.6).

See 17 for PATIENT COUNSELING INFORMATION and Medication Guide.

### Full Prescribing Information Boxed Warning

### Thrombotic Events

Serious thrombotic events have occurred in fitusiran-treated patients with risk factors for thromboembolism including persistent antithrombin (AT) activity less than 15%, use of fitusiran 80 mg once monthly, presence of indwelling venous catheters, and in the post-operative setting when bleed management guidelines were not followed.

Monitor AT activity using an FDA-cleared test and target AT activity 15-35% to reduce the risk of thrombosis. Monitor patients for signs and symptoms of thrombotic events. Interrupt fitusiran in patients with a thrombotic event and manage as clinically indicated. (5.1)

### Acute and Recurrent Gallbladder Disease

Acute and recurrent gallbladder disease, including cholelithiasis and cholecystitis have occurred in fitusiran-treated patients, some of whom required cholecystectomy or had complications (e.g., pancreatitis) related to gallbladder disease. Monitor patients for signs and symptoms of acute and recurrent gallbladder disease.

Consider interruption or discontinuation of fitusiran if gallbladder disease occurs. Consider alternative treatment for hemophilia in patients with a history of symptomatic gallbladder disease. (5.2)

### 1 Indications and Usage

Fitusiran is indicated for routine prophylaxis to prevent or reduce the frequency of bleeding episodes in adult and pediatric patients aged 12 years and older with hemophilia A or B with or without factor VIII or IX inhibitors.

### 2 Dosage and Administration

### 2.1 Recommended Dosage

For subcutaneous use only.

Use of fitusiran is recommended under the supervision of a healthcare professional experienced in the treatment of hemophilia or bleeding disorders.

Monitor AT activity using an FDA-cleared test. Information on FDA-cleared tests for AT activity is available at http://www.fda.gov/CompanionDiagnostics.

Measure AT activity prior to initiation of fitusiran. Do not initiate fitusiran dosing if AT activity is <60%.

After fitusiran is initiated, patients may continue their prior clotting factor concentrates (CFC) or bypassing agent (BPA) prophylaxis for the first 7 days of treatment. Discontinue CFC or BPA prophylaxis no later than 7 days after the initial dose of fitusiran.

The starting dose of fitusiran is 50 mg once subcutaneously every two months. Adjust the dose and/or dosing interval, if needed, to maintain AT activity between 15-35% [see Boxed Warning, Dosage and Administration (2.2) and Warnings and Precautions (5.1)].

### 2.2 Dosage Modification

Measure AT activity using an FDA-cleared test at Weeks 4 (Month 1), 12 (Month 3), 20 (Month 5) and 24 (Month 6) following the starting dose and after any dose modification.

If any AT activity is <15%, a dose reduction is required. The lower dose should be initiated 3 months after the prior dose. AT measurements should be restarted after a dose reduction.

If AT activity is >35% after 6 months, or if the patient has not achieved satisfactory bleed control, dose escalation should be considered. AT measurements should be restarted after a dose escalation.

Refer to Table 2 above for modified dosage based on AT activity levels.

Once the patient's target dose is identified based on AT activity 15-35%, measure AT activity annually. Additional AT measurements can be considered if bleeding control is not adequate.

After cessation of fitusiran dosing, routine AT monitoring is not needed unless the patient is bleeding and treatment with CFC/BPA is required. Based on data from the clinical studies, a majority of patients have AT activity >60% by 6 months after the last fitusiran dose, after which standard doses of CFC/BPA may be used.

### Missed Dose

If a dose of fitusiran is missed, administer as soon as possible; thereafter, resume the patient's usual dosing schedule of either once every month or once every two months, as applicable, from the last dose.

### 2.3 Bleed Management

### Breakthrough Bleed Management

If breakthrough bleeding requiring on-demand treatment with CFC or BPA occurs during the first 7 days after fitusiran initiation, manage the bleed using the patient's prior dosing regimen of CFC or BPA.

If breakthrough bleeding occurs after 7 days from the first fitusiran dose, bleeds should be managed with a reduced dose and frequency of CFC/BPA to minimize the risk of thrombotic events. Initially, the weight-based dose of a CFC/BPA should be reduced, and the dosing interval doubled compared to the standard dose. This reduced dosing is shown in Table 4 above. If adequate hemostatic control is not achieved, higher doses may be used based on clinical judgement. Combination use of antifibrinolytics with CFC or BPA has not been studied.

### Use of Fitusiran During Surgical Interventions

In clinical studies, patients with hemophilia A or B with or without inhibitors have undergone both major (N=60) and minor (N=71) surgical procedures without discontinuing fitusiran prophylaxis. Utilize bleed management guidelines during the perioperative period for hemostatic management.

### 2.4 Preparation and Administration Instructions

Fitusiran drug product is intended for use under the guidance of a healthcare provider. Provide proper training to patients and/or caregivers on the preparation and administration of fitusiran prior to use, according to the Instructions for Use (IFU). A patient may self-inject fitusiran drug product or the patient's caregiver may administer fitusiran drug product. In pediatric patients 12 to 17 years of age, it is recommended that fitusiran drug product be administered by or under the supervision of an adult.

Fitusiran drug product for subcutaneous administration is a clear, colorless to pale yellow solution. Do not use if the solution is discolored or cloudy, or if it contains visible flakes or particles. Do not use fitusiran drug product if it has been dropped or damaged.

If fitusiran drug product is stored at room temperature, it is ready for use. If fitusiran drug product is stored in the refrigerator, remove from the refrigerator and allow fitusiran drug product to reach room temperature, for at least 30 minutes.

Administer fitusiran drug product by subcutaneous injection in the thigh or abdomen region, except for the 2 inches (5 cm) around the navel. A caregiver can also inject fitusiran drug product in the outer area of the patient's upper arm. Fitusiran drug product should not be injected into skin that is tender, damaged, bruised, or scarred. Do not inject into a vein.

To inject 10 mg or 20 mg of fitusiran from the single-dose vial, it is recommended to use a sterile 1 mL Luer Lock syringe (polypropylene or polycarbonate), and a sterile 27 gauge ½ inch (13 mm) Luer Lock needle to withdraw and inject fitusiran drug product solution subcutaneously (see the Instructions for Use).

Discard unused product remaining in the prefilled pen or vial.

### 3 Dosage Forms and Strengths

Fitusiran drug product is a clear, colorless to pale yellow solution in a single-dose:
Injection: 50 mg/0.5 mL prefilled pen
Injection: 20 mg/0.5 mL prefilled pen
Injection: 20 mg/0.2 mL vial

### 4 Contraindications

None.

### Warnings and Precautions

### 5.1 Thrombotic Events

Serious thrombotic events have been reported in fitusiran-treated patients. Thrombotic events were reported in 2.6% of patients receiving the 80 mg once monthly dose (2.3 events per 100 person-years), including a fatal event of cerebral venous sinus thrombosis. The 80 mg once monthly dose is not approved or recommended for use. Thrombotic events were reported in 1.4% of patients receiving fitusiran prophylaxis using the antithrombin-based dose regimen (AT-DR) that targeted AT activity 15-35% (0.8 events per 100 person-years). Participants with established thrombophilia or a history of thrombosis were generally excluded from studies with fitusiran.

The risk of thrombosis is greater in patients with persistent AT activity <15%, with comorbidities that predispose to thrombosis, when bleed management guidelines are not followed in the post-operative setting, when there is an indwelling venous catheter, and with use of the 80 mg once monthly (non-AT-based) dose. Treatment of breakthrough bleeding episodes with CFC or BPA at a dose greater or more frequent than recommended may also increase thrombotic risk [see Dosage and Administration (2.3)]. The decision to utilize higher dosing regimens of CFC or BPA in the setting of inadequate hemostasis requires an assessment of the benefits and risks and close clinical monitoring.

Monitor AT activity using an FDA-cleared test and target AT activity 15-35% to reduce the risk of thrombosis [see Dosage and Administration (2.1, 2.2) and Warnings and Precautions (5.1)]. Monitor patients for signs and symptoms of thrombotic events. Interrupt fitusiran prophylaxis in patients with a thrombotic event and manage as clinically indicated.

Inform patients treated with fitusiran to monitor for and report signs and symptoms of thrombotic events. Consider the benefits and risks of resuming fitusiran prophylaxis following resolution of the thrombotic event.

### 5.2 Acute and Recurrent Gallbladder Disease

Treatment with fitusiran is associated with an increased occurrence of acute and recurrent gallbladder disease including cholelithiasis and cholecystitis. Fitusiran at a fixed dose (including 80 mg once monthly) is not approved or recommended for use. In the 270 patients in the fitusiran clinical studies who received the fixed dose (non-AT-based dose) once monthly regimen, 17% experienced gallbladder events and 4% (11 patients) underwent cholecystectomy.

In 286 patients who received the AT-DR, 3.8% experienced gallbladder events and 0.3% (1 patient) underwent cholecystectomy.

All but one of the patients who underwent cholecystectomy resumed fitusiran after surgery. One patient who started on fixed dosing experienced cholangitis and pancreatitis caused by gallstone disease more than a year after cholecystectomy while receiving AT-DR.

Patients diagnosed with acute or recurrent gallbladder disease most commonly presented with epigastric pain, generalized abdominal pain, indigestion, nausea and/or vomiting. If gallbladder disease is suspected, appropriate imaging and clinical follow-up are indicated.

Consider alternative treatment for hemophilia in patients with a history of symptomatic gallbladder disease. Consider interruption or discontinuation of fitusiran if gallbladder disease occurs.

### 5.3 Hepatotoxicity

In the two randomized studies testing fitusiran 80 mg once monthly, serum alanine transaminase (ALT) and aspartate transaminase (AST) elevations above 3 times the upper limit of normal (ULN) occurred in 32% of patients with hemophilia with inhibitors and 18% of patients with hemophilia without inhibitors compared to no events of AST or ALT elevation greater than 3×ULN in the control groups. There was one case of moderate hepatic injury (ALT elevation >300 U/L and total serum bilirubin >3 mg/dL) attributable to fitusiran use. This patient had elevation of liver tests after a single 80 mg dose that continued to rise with repeated dosing of fitusiran 80 mg once monthly. This patient's liver tests recovered with drug discontinuation. Fitusiran 80 mg once monthly is not approved or recommended for use.

On the AT-DR, 3.4% of patients treated with fitusiran had at least one ALT value greater than 3×ULN with a median onset of 89 days after initial dosing (range 15 to 768 days).

Avoid use of fitusiran in patients with hepatic impairment (Child-Pugh Class A, B and C).

Obtain baseline liver tests including AST, ALT, and total bilirubin prior to initiating fitusiran, monthly for at least the first 6 months of fitusiran use, and monthly for at least 6 months after a dose increase, and periodically thereafter as clinically indicated.

If new or worsening liver test abnormalities occur, perform appropriate diagnostic evaluations, initiate medical management as appropriate and monitor laboratory parameters until they return to baseline. If ALT or AST elevations greater than 5×ULN occur, interrupt fitusiran treatment. Consider the benefits and risks of resuming fitusiran prophylaxis following resolution of transaminase elevations. If you decide to restart fitusiran, wait until liver tests have returned to baseline. If fitusiran is restarted and ALT or AST elevations greater than 5×ULN reoccur or the patient experiences jaundice (total bilirubin >2.5 mg/dL) thought to be from hepatotoxicity with other causes of liver test elevation ruled out, permanently discontinue fitusiran.

### 6 Adverse Reactions

The following clinically significant adverse reactions are described elsewhere in the labeling:
Thrombotic Events [see Boxed Warning and Warnings and Precautions (5.1)]
Acute and Recurrent Gallbladder Disease [see Boxed Warning and Warnings and Precautions (5.2)]
Hepatotoxicity [see Warnings and Precautions (5.3)]

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

The pooled safety population described in the WARNINGS AND PRECAUTIONS reflect exposure to fitusiran as fixed doses and AT-DR (N=335).

The safety of the fitusiran AT-DR was assessed in 286 adult and pediatric male patients with hemophilia A or B with or without inhibitors [see Clinical Studies (14)].

Among patients who received the AT-DR, 93% were exposed for 6 months or longer and 83% were exposed for 12 months or longer. The median duration of exposure across the studies was 674 days (with a maximum of 896 days).

Serious adverse reactions occurred in 4/286 (1.4%) patients who received the AT-DR, two of whom had serious adverse reactions of cholecystitis.

Permanent discontinuation of fitusiran due to an adverse reaction occurred in 4/286 (1.4%) patients receiving the AT-DR and included liver injury, post-operative deep vein thrombosis, cerebral infarction and pruritus.

Dosage interruptions of fitusiran due to an adverse reaction occurred in 2/286 (0.7%) patients receiving the AT-DR and included increased serum transaminases.

The most common adverse reactions (>10%) reported in patients treated with the AT-DR were viral infection, nasopharyngitis, and bacterial infection.

TABLE 5
Adverse Reactions Reported in ≥5% of Patients
from Pooled Clinical Studies with Fitusiran AT-DR

| | Number of Patients |
|---|---|
| | (%) |
| Adverse Reaction | N = 286 |
| Viral infection* | 29 |
| Nasopharyngitis* | 26 |
| Bacterial infection* | 11 |
| Hepatic Injury^{†} | 8 |
| Arthralgia* | 8 |
| Prothrombin fragment 1.2 increased | 7 |
| Injection site reaction^{‡} | 6 |
| Headache | 5 |
| Cough* | 5 |

*Includes similar terms.
^{†}Hepatic injury includes: alanine aminotransferase increased, aspartate aminotransferase increased, liver injury.
^{‡}injection site reactions: includes injection site bruising, injection site erythema, injection site pain, injection site hematoma, injection site atrophy, injection site hemorrhage, injection site discomfort, injection site swelling, injection site discoloration, injection site pruritus, injection site induration, injection site nodule, injection site mass, injection site vesicles, injection site deformation, injection site rash, injection site joint pain and application site erythema.

Clinically relevant adverse reactions in less than 5% of patients include:
Dyspepsia
Abdominal pain

### 7 Drug Interactions

7.1 Hypercoagulability with Concomitant Use of CFC or BPA

Fitusiran prophylaxis leads to increased thrombin generation with additive increase in peak thrombin when used concomitantly with CFC or BPA [see Boxed Warning, Dosage and Administration (2.3) and Warnings and Precautions (5.1)].

### 8 Use in Specific Populations

### 8.1 Pregnancy

### Risk Summary

There are no available data on fitusiran use in pregnant women to evaluate for a drug-associated risk of major birth defects, miscarriage, or other adverse maternal or fetal outcomes. Reproduction studies in pregnant animals have not been conducted with fitusiran. It is not known whether fitusiran can cause fetal harm when administered to a pregnant woman or can affect reproduction capacity. Fitusiran should be used during pregnancy only if the potential benefit justifies the potential risks, including those to the fetus.

The estimated background risk of major birth defects and miscarriage for the indicated population is unknown. All pregnancies have a background risk of birth defect, loss, or other adverse outcomes. In the U.S. general population, the estimated background risk of major birth defects and miscarriage in clinically recognized pregnancies is 24% and 15-20%, respectively.

### 8.2 Lactation

### Risk Summary

There are no data on the presence of fitusiran or its metabolite in human milk, the effects on the breastfed child, or the effects on milk production. It is not known whether fitusiran is safe for use during breastfeeding. The developmental and health benefits of breastfeeding should be considered along with the mother's clinical need for fitusiran and any potential adverse effects on the breastfed infant from fitusiran or from the underlying maternal condition.

### 8.3 Females and Males of Reproductive Potential

### Contraception—Females

Use of fitusiran in women using hormonal contraceptives may increase the risk of thrombotic events. Estrogen based hormonal contraceptives are an established risk factor for thrombosis in women with inherited AT deficiency. Advise patients using hormonal contraceptives to use an alternative non-hormonal contraception prior to starting treatment with and while receiving fitusiran.

### 8.4 Pediatric Use

The safety and effectiveness of fitusiran for the treatment of hemophilia A or B with or without factor VIII or IX inhibitors have been established in pediatric patients aged 12 years and older. Use of fitusiran in pediatric patients with hemophilia A and B is supported by evidence from adequate and well-controlled studies in adult and pediatric patients [see Adverse Reactions (6.1) and Clinical Studies (14)]. A total of 60 pediatric patients ages 12 to 17 were treated with fitusiran in the clinical studies.

The safety and effectiveness of fitusiran have not been established in pediatric patients below 12 years of age.

### 8.5 Geriatric Use

There were 3 patients with hemophilia 65 years of age and older in the clinical studies on fitusiran. Clinical studies of fitusiran did not include sufficient numbers of subjects aged 65 and over to determine whether they respond differently from younger subjects.

### 8.6 Hepatic Impairment

Serum transaminase elevations have been observed in the clinical studies [see Warnings and Precautions (5.3)]. Avoid use of fitusiran in patients with established hepatic impairment (Child-Pugh Class A, B and C).

### 11 Description

Fitusiran drug product for injection contains fitusiran, an antithrombin-directed double-stranded small interfering ribonucleic acid (siRNA), which is covalently linked to a ligand containing a triantennary N-acetylgalactosamine (GalNAc) moiety. The molecular formula of fitusiran sodium is C₅₂₀H₆₃₆F₂₁N₁₇₅Na₄₃O₃₀₉P₄₃S₆ and the molecular weight is 17,193 Da. Fitusiran drug substance is a white to pale yellow powder and freely soluble in water and phosphate buffered saline. The pH of a 1% aqueous solution of fitusiran drug substance in 50 mM KCl is 5.4 (4.4-7.3). Its structural formula is shown in FIG. 1.

Fitusiran drug product is supplied as a sterile, preservative-free, clear, colorless to pale yellow solution for subcutaneous administration.

Each 50 mg single-dose prefilled pen delivers 50 mg fitusiran (equivalent to 53.0 mg fitusiran sodium) in 0.5 mL, and also contains dibasic sodium phosphate (0.585 mg), monobasic sodium phosphate (0.044 mg), sodium chloride (2.455 mg), and Water for Injection, USP. Phosphoric acid (concentrated) and sodium hydroxide may be added to adjust to pH 7.0.

Each 20 mg single-dose prefilled pen delivers 20 mg fitusiran (equivalent to 21.2 mg fitusiran sodium) in 0.5 mL, and also contains dibasic sodium phosphate (0.467 mg), monobasic sodium phosphate (0.105 mg), sodium chloride (3.836 mg), and Water for Injection, USP. Phosphoric acid (concentrated) and sodium hydroxide may be added to adjust to pH 7.0.

Each 20 mg single-dose vial delivers 20 mg fitusiran (equivalent to 21.2 mg fitusiran sodium) in 0.2 mL, and also contains dibasic sodium phosphate (0.234 mg), monobasic sodium phosphate (0.018 mg), sodium chloride (0.982 mg), and Water for Injection, USP. Phosphoric acid (concentrated) and sodium hydroxide may be added to adjust to pH 7.0.

### 12 Clinical Pharmacology

### 12.1 Mechanism of Action

Fitusiran is a double-stranded siRNA that causes degradation of AT messenger RNA (mRNA) through RNA interference, reducing plasma AT levels.

### 12.2 Pharmacodynamics

In clinical studies with fitusiran in hemophilia patients, the primary pharmacodynamic (PD) measure was plasma AT activity. Lower AT activity levels were associated with lower annualized bleeding rates (ABR); however, persistent AT activity <15% is a risk factor for thrombotic events.

In Study LTE15174, the mean (SD) AT activity on the AT-DR was 24% (4.64).

Simulations to estimate time for AT activity to reach steady state found that regardless of the dose regimen, more than 99% of participants reached steady state AT levels (defined as <10% variability among consecutive measures) within 23 weeks following initiation of fitusiran dosing or following dose escalation or de-escalation. The duration of persistent AT activity <60% (non-negligible impact of fitusiran on coagulation) following fitusiran treatment discontinuation was estimated to be approximately 6 months, after which standard doses of CFC/BPA may be administered. In patients with AT activity <15%, the time required prior to initiation of a lower dose was estimated to be 12 weeks to maximize the AT activity recovery to the target range of 15% to 35%. Extent of AT reduction with fitusiran did not differ between patients with hemophilia A or B, with or without inhibitors.

### 12.3 Pharmacokinetics

The pharmacokinetic (PK) properties of fitusiran were evaluated following administration of fitusiran in patients with hemophilia A or B, with or without inhibitors as summarized in Table 6. Fitusiran PD is driven by liver PK rather than plasma PK. The fitusiran dosing strategy is based on maintaining plasma AT activity levels between 15-35% with 10, 20 or 50 mg dosing every month or every two months.

TABLE 6
Pharmacokinetic Parameters of Fitusiran

| | | |
|---|---|---|
| PK Parameters* | Fitusiran 20 mg | Fitusiran 50 mg |

General Information

| Steady State Exposure: | | | | |
|---|---|---|---|---|
| Cₘₐₓ (ng/mL) | 34.4 | (10.1) [29%] | 84.1 | (58.7) [70%] |
| AUC (ng · h/mL) | 491 | (83.8) [17%] | 1290 | (377) [29%] |

| | |
|---|---|
| Dose Proportionality | Fitusiran exhibited a dose proportional increase in plasma |
| | exposure after SC administration. |
| Accumulation^{†} | No accumulation of fitusiran was observed following multiple |
| | once monthly dosing. |

Absorption

| | | | | |
|---|---|---|---|---|
| tₘₐₓ (h) | 2.88 | (0.5-4.33) | 3.78 | (0.42-11.9) |

Distribution^{‡}

| | | | | |
|---|---|---|---|---|
| Vss/F | 431 | (135) [31%] | 570 | (713) [125%] |

| | |
|---|---|
| Protein Binding^{§} | 96.6% at clinically relevant exposure (0.5 microgram/ml) |

Elimination

| | | | | |
|---|---|---|---|---|
| t_{1/2} (h) | 5.57 | (2.36) [42%] | 7.98 | (4.74) [59%] |
| CL/F | 41.9 | (8.39) [20%] | 50.8 | (71.1) [140%] |

Metabolism

| | |
|---|---|
| Primary Pathway | Fitusiran is metabolized by endo- and exo-nucleases to |
| | oligonucleotides of varying lengths, which are further |
| | metabolized to oligonucleotides of even shorter lengths. |
| | Fitusiran is not a substrate for CYP450 or transporters. |
| Main Metabolite | AS(N-1)3′ |

Excretion

| | | |
|---|---|---|
| Primary Pathway | Not evaluated | 14.6% (mean) of the |
| | | administered 50 mg dose of |
| | | fitusiran is recovered unchanged |
| | | in 0-24 hour urine |

AUC = area under the plasma concentration-time curve extrapolated to infinity;
CL/F = apparent clearance;
Cₘₐₓ = maximum plasma concentration;
CV = coefficient of variation;
PK = pharmacokinetic(s);
SD = standard deviation;
t_{1/2} = terminal elimination half-life;
tₘₐₓ = time to maximum concentration;
Vss/F = apparent volume of distribution
*Unless otherwise indicated, the PK parameters in this table are from Study LTE15174. All PK parameters are estimated using non-compartmental analysis and are presented as Mean (SD) [CV %], except for tₘₐₓ which is presented as median (range)
^{†}Study TDR14767 (ALN-AT3SC-001)
^{‡}Fitusiran distributes primarily to the liver after subcutaneous injection.
^{§}In vitro data

### Specific Populations

Fitusiran has only been studied in male patients. The pharmacokinetics of fitusiran is not influenced by race. Fitusiran has not been studied in children <12 years old.

No clinical study to evaluate the effect of hepatic impairment or renal impairment on the pharmacokinetics of fitusiran was conducted. In the pivotal studies, 12 patients had mild renal impairment (eGFR:60 to <90 mL/min/1.73 m2) and 1 patient had moderate renal impairment (eGFR 30 mL/min/1.73 m2 to <60 mL/min/1.73 m2). Population PK analysis indicated no impact of renal impairment on the exposure (Cₘₐₓ and AUC) of fitusiran in patients with mild renal impairment.

### Drug Interaction Studies

### Clinical Studies

No clinical drug-drug interaction studies have been conducted to evaluate the potential of fitusiran to interact with other co-administered drugs which are either substrates, inhibitors or inducers of CYP isozymes, or are substrates or inhibitors of drug transporters.

### In Vitro Studies

Results from in vitro studies evaluating the potential of co-administered drugs to increase the PK exposure of fitusiran, and the potential of fitusiran to increase the PK exposure of co-administered drugs, indicate that CYP- or transporter-mediated interactions between fitusiran and co-administered drugs are unlikely at clinically relevant concentrations.

### 12.6 Immunogenicity

The observed incidence of anti-drug antibodies (ADA) is highly dependent on the sensitivity and specificity of the assay. Differences in assay methods preclude meaningful comparisons of the incidence of ADA in the studies described below with the incidence of ADA in other studies, including those of fitusiran or of other fitusiran products.

During fitusiran treatment (up to 250 weeks) in four studies, 10 out of 290 adults with hemophilia (3.4%) developed ADAs. All patients had low ADA titers and the majority of patients had transient ADAs. There was no identified clinically significant effect of ADAs on PK, PD, safety, or effectiveness of fitusiran.

### 13 Nonclinical Toxicology

### 13.1 Carcinogenesis, Mutagenesis, Impairment of Fertility

In a 2-year carcinogenicity study in Sprague Dawley rats, fitusiran was not carcinogenic up to the highest dose tested following subcutaneous administration at doses of 0.03, 0.1 or 0.3 mg/kg/week in males and 0.1, 0.3, or 1 mg/kg/week in females. All doses in males and females were below the maximum recommended human dose (MRHD) of 50 mg per month based on body surface area (BSA) comparisons due to the sensitivity of wild type animals to the pharmacological effects of fitusiran. Fitusiran was not carcinogenic in Tg-rasH2 mice following subcutaneous administration at doses of 0.05, 0.15, or 0.5 mg/kg/week in males and 0.025, 0.08, or 0.25 mg/kg/week in females for 26 weeks.

Fitusiran was not mutagenic or clastogenic in a battery of genetic toxicity studies (i.e., in vitro bacterial reverse mutation assay, in vitro chromosomal aberration assay in human peripheral blood lymphocytes; in vivo bone marrow micronucleus test in rats).

In a chronic repeat dose toxicology study in Sprague Dawley rats, fitusiran-treated males received subcutaneous doses of 0.25, 0.5, or 1 mg/kg/week for 16 weeks then were cohabitated with fitusiran-naïve females. No adverse effects on male fertility endpoints were observed. All doses used in males were below the MRHD based on BSA comparisons due to the sensitivity of wild type animals to the pharmacological effects of fitusiran.

### 14 Clinical Studies

The efficacy and safety of fitusiran in adult and pediatric patients aged 12 years and older with hemophilia A or B with or without inhibitors were established in two clinical studies: Hemophilia A or B with Inhibitory Antibodies: ATLAS-INH (NCT03417102) Hemophilia A or B without Inhibitory Antibodies: ATLAS-A/B (NCT03417245)

Patients in the above parent studies rolled over into the long-term extension study ATLAS-OLE (NCT03754790).

The clinical studies ATLAS-INH and ATLAS-A/B tested an 80 mg monthly fixed dose of fitusiran. Because of thrombotic events with this dose, the fitusiran AT-DR targeting AT activity of 15-35% was implemented in ATLAS-OLE. The AT-DR was initiated when studies ATLAS-INH and ATLAS-A/B were nearly completed, therefore, the efficacy of fitusiran AT-DR treatment was assessed by comparing the fitusiran AT-DR treatment data from the long-term extension study ATLAS-OLE to the control data from studies ATLAS-INH and ATLAS-A/B. The efficacy analyses were conducted according to the intent to treat (ITT) principle preserving the randomization in the parent studies.

### ATLAS-INH

ATLAS-INH was a randomized, multicenter, open-label clinical study in 57 adult and pediatric males (aged >12 years) with hemophilia A or B with inhibitory antibodies to factor VIII (FVIII) or factor IX (FIX), who previously received on-demand (episodic) treatment with BPAs for bleeding. Eligible patients were randomized in a 2:1 ratio to receive fitusiran prophylaxis at a fixed dose 80 mg SC monthly (N=38) or BPA on-demand for treatment of breakthrough bleeding episodes (N=19) for 9 months. The 80 mg dose of fitusiran is not approved because of an increased risk of serious thrombotic events, gallbladder events (including the need for cholecystectomy) and hepatotoxicity [see Boxed Warning and Warnings and Precautions (5.1, 5.2, 5.3)].

Of the 57 enrolled patients all had inhibitors; 45 patients had Hemophilia A and 12 had Hemophilia B. All patients in the study were male. The mean age of patients was 28.4 years, and 10 (17.5%) patients were 12-17 years of age. A total of 68.4% of patients were Asian, 28.1% were White, 1.8% were other, and 1.8% were multiple races; 5.3% patients identified as Hispanic or Latino and 94.7% identified as not Hispanic or Latino. Generally, the demographic and patient characteristics at baseline were comparable between the patients with hemophilia A and B.

### ATLAS A/B

ATLAS A/B was a randomized, multicenter, open-label clinical study in 120 adult and pediatric males (aged >12 years) with hemophilia A or B without inhibitory antibodies to FVIII or FIX, who previously received on-demand (episodic) treatment with CFC for bleeding. Eligible patients were randomized in a 2:1 ratio to receive fitusiran prophylaxis at a fixed dose of 80 mg SC monthly (N=80) or CFCs on-demand to treat breakthrough bleeding episodes (N=40) for 9 months. The 80 mg dose of fitusiran is not approved because of an increased risk of serious thrombotic events, gallbladder events (including the need for cholecystectomy) and hepatotoxicity [see Boxed Warning and Warning and Precautions (5.1, 5.2, 5.3)].

Of the 120 enrolled patients none had inhibitors; 93 patients had Hemophilia A and 27 had Hemophilia B. All patients in the study were male. The mean age of patients was 33.8 years, and 14 (11.7%) patients were 12-17 years of age. A total of 59.2% of patients were Asian, 37.5% were White, 1.7% were Black or African American, and 1.7% were multiple races; 3.3% of patients identified as Hispanic or Latino, 90.8% identified as not Hispanic or Latino, and 5.8% had ethnicity information unreported. Generally, the demographic and patient characteristics at baseline were comparable between the patients with hemophilia A and B.

### ATLAS-OLE

A total of 227 patients rolled over from two clinical studies (ATLAS-INH and ATLAS-A/B) and ATLAS-PPX, a crossover study in patients previously on CFC or BPA prophylaxis, and were treated with fitusiran in ATLAS-OLE. This multicenter open-label extension study evaluated the long-term safety and efficacy of fitusiran in adult and pediatric males aged >12 years with hemophilia A or B, with or without inhibitory antibodies to FVIII or FIX. Eligible patients initially received fitusiran 80 mg SC once monthly. The study was amended to evaluate the efficacy and safety of the AT-DR. A total of 213 patients were subsequently transitioned to AT-DR targeting AT activity of 15-35%.

In the AT-DR, the fitusiran starting dose was 50 mg every two months, and dosing was individually adjusted based on AT activity level using the INNOVANCE Antithrombin assay. The dose could be increased to 50 mg every month or 80 mg every month or decreased to 20 mg every two months or 20 mg every month. Fitusiran was discontinued if AT activity was <15% at the lowest dose. No patients required escalation to 80 mg every month to achieve the target AT range. The dose required to maintain AT activity 15-35% in patients who initiated dosing on 50 mg every two months was: 50 mg every two months (35.8% of patients), 50 mg every month (15.7% of patients), 20 mg every two months (30.9% of patients), or 20 mg every month (2.9% of patients). A total of 14.7% of patients discontinued fitusiran due to more than one AT activity <15%.

Patients with known co-existing coagulation disorders other than hemophilia A or B, increased risk of thrombosis as assessed by history of arterial or venous thromboembolism, significant valvular disease or atrial fibrillation, or co-existing thrombophilic disorder (e.g., Factor V Leiden mutation), AT activity <60% at screening, platelet count <100,000/L, eGFR <45 mL/min (using the MDRD), or clinically significant liver disease were not eligible for enrollment.

The efficacy of fitusiran AT-DR in ATLAS-OLE was evaluated for a duration of 7 months (primary efficacy period) following a 6-month dose adjustment period. The median observed annualized bleeding rate (IQR) for treated bleeds was 3.7 (0.0; 7.5) overall, 1.9 (0.0; 5.6) in inhibitor patients and 3.8 (0.0; 11.2) in non-inhibitor patients.

### Fitusiran Prophylaxis Compared to On-Demand BPA or CFC

The efficacy results of fitusiran prophylaxis using AT-DR in ATLAS-OLE compared to on-demand BPA or CFC control data from studies ATLAS-INH and ATLAS-A/B with respect to rate of treated bleeds, treated spontaneous bleeds, and treated joint bleeds are shown in Table 7 (patients with inhibitors) and Table 8 (patients without inhibitors) below.

TABLE 7
Annualized Bleed Rate for Treated Bleeds with
Fitusiran Prophylaxis versus On-Demand BPA
in Patients ≥12 Years of Age with Inhibitors

| | | |
|---|---|---|
| | Fitusiran AT-DR | On-Demand BPA |
| Endpoint | (N = 38) | (N = 19) |

All Treated Bleeds

| | | | |
|---|---|---|---|
| ABR (95% CI)* | 5.1 (2.8, 9.5) | 19.1 | (11.8, 31.0) |

| | |
|---|---|
| % reduction | 73% |
| p-value | p = 0.0006 |

Treated Spontaneous Bleeds

| | | | |
|---|---|---|---|
| ABR (95% CI)* | 3.1 (1.8, 5.4) | 17.1 | (9.9, 29.6) |

| | |
|---|---|
| % reduction | 82% |
| p-value | <0.0001 |

Treated Joint Bleeds

| | | | |
|---|---|---|---|
| ABR (95% CI)* | 4.0 (2.5, 6.2) | 14.4 | (9.0, 23.1) |

| | |
|---|---|
| % reduction | 73% |
| p-value | p = 0.0001 |

ABR = annualized bleed rate; AT-DR = AT-Dosing Regimen; BPA = bypassing agents; CI = confidence interval.
*Based on negative binomial regression model.

TABLE 8
Annualized Bleed Rate for Treated Bleeds with
Fitusiran Prophylaxis versus On-Demand CFC in
Patients ≥12 Years of Age without Inhibitors

| | | |
|---|---|---|
| | Fitusiran AT-DR | On-Demand CFC |
| Endpoint | (N = 80) | (N = 40) |

All Treated Bleeds

| | | | |
|---|---|---|---|
| ABR (95% CI)* | 9.0 | (5.6, 14.5) | 31.4 (20.5, 48.2) |

| | |
|---|---|
| % reduction | 71% |
| p-value | p < 0.0001 |

Treated Spontaneous Bleeds

| | | | |
|---|---|---|---|
| ABR (95% CI)* | 5.4 | (3.7, 8.0) | 21.0 (14.0, 31.6) |

| | |
|---|---|
| % reduction | 74% |
| p-value | p < 0.0001 |

Treated Joint Bleeds

| | | | |
|---|---|---|---|
| ABR (95% CI)* | 6.2 | (4.2, 9.2) | 21.6 (14.6, 31.9) |

| | |
|---|---|
| % reduction | 71% |
| p-value | p < 0.0001 |

ABR = annualized bleed rate; AT-DR = AT-Dosing Regimen; CFC = clotting factor concentrate; CI = confidence interval.
*Based on negative binomial regression model.

### 16 How Supplied/Storage and Handling

### 16.1 How Supplied

Fitusiran drug product is a clear, colorless to pale yellow solution supplied in a single-dose prefilled pen or a single-dose vial. Each prefilled pen is designed to deliver:
50 mg of fitusiran in 0.5 mL (NDC 58468-0348-1).
20 mg of fitusiran in 0.5 mL.

Each vial is designed to deliver 20 mg of fitusiran in 0.2 mL (NDC 58468-0347-1).

Fitusiran drug product is available in cartons containing 1 prefilled pen or 1 vial.

### Storage and Handling

### 50 mg Prefilled Pen

Store Prefilled Pen refrigerated at 2° C. to 8° C. (36° F. to 46° F.) in the original carton to protect from light.

Prefilled Pen may be stored at room temperature between 15° C. to 30° C. (59° F. to 86° F.) for a single period of up to 3 months within the expiration date printed on the label. Discard no later than 3 months after removal from the refrigerator or at the expiration date, whichever comes first. After storage at room temperature, do not return the product to the refrigerator.

### 20 mg Prefilled Pen

Store Prefilled Pen refrigerated at 2° C. to 8° C. (36° F. to 46° F.) in the original carton to protect from light.

Prefilled Pen may be stored at room temperature between 15° C. to 30° C. (59° F. to 86° F.) for a single period of up to 3 months within the expiration date printed on the label. Discard no later than 3 months after removal from the refrigerator or at the expiration date, whichever comes first. After storage at room temperature, do not return the product to the refrigerator.

### 20 mg Vial

Store Vial either in the refrigerator at 2° C. to 8° C. (36° F. to 46° F.) or at room temperature between 15° C. to 30° C. (59° F. to 86° F.), in the original carton to protect from light. After storage at room temperature, do not return the product to the refrigerator.

Do not shake fitusiran Prefilled Pen or Vial at any time. Do not heat fitusiran Prefilled Pen or Vial. Do not freeze fitusiran Prefilled Pen or Vial. Do not put fitusiran Prefilled Pen or Vial into direct sunlight.

### 17 Patient Counseling Information

Advise the patient to read the FDA-approved patient labeling (Medication Guide and Instructions for Use).

### Use of BPAs or CFCs

Advise the patient and/or caregiver to discontinue prophylactic use of BPA or CFC no later than 7 days after starting fitusiran to reduce the risk of thrombotic events. Discuss the appropriate dosing and frequency of BPA or CFC for breakthrough bleed management with the patient and/or caregiver prior to starting fitusiran prophylaxis [see Dosage and Administration (2.3)].

### Thrombotic Events

Advise the patient and/or caregiver of the risk of thrombotic events while receiving fitusiran. Inform the patient and/or caregiver of the need for periodic measurements of AT activity that may result in changes to the fitusiran dose and/or frequency of administration to reduce the risk for thrombosis. Educate patients on the signs and symptoms of thrombotic events and to seek immediate medical attention if new symptoms of thrombotic events occur [see Warnings and Precautions (5.1)].

### Acute and Recurrent Gallbladder Disease

Inform the patient and/or caregiver of the risk of acute and recurrent gallbladder disease while receiving fitusiran. Educate patients on the signs and symptoms of gallbladder disease and to seek medical attention if new symptoms of gallbladder disease occur [see Warnings and Precautions (5.2)].

### Hepatotoxicity

Inform the patient and/or caregiver of the risk of hepatotoxicity and that blood tests to monitor for this risk will be obtained before starting fitusiran and periodically during treatment. Inform patients to seek medical attention if symptoms of hepatotoxicity occur [see Warnings and Precautions (5.3)].

### Administration Instructions

Provide training to the patient and/or caregiver on proper subcutaneous injection technique, including aseptic technique, and the preparation and administration of fitusiran prior to use [see Dosage and Administration (2.4)].

### Example 2: Selection of a 10 mg De-Escalation Dose for Fitusiran

This Example describes the selection of a 10 mg de-escalation dose and simulated responses for virtual patients.

Simulations of Fitusiran Dose Regimens Identify Participant Distribution within Different AT Activity Ranges

As described in Example 1, in the ATLAS-OLE Phase 3 open-label extension study, the fitusiran antithrombin-based dose regimen (AT-DR) targeting AT levels of 15-35% was well tolerated and maintained clinically meaningful bleed protection. However, 11.7% of participants discontinued fitusiran due to persistent AT levels <15% on the lowest dose (20 mg every two months; Q2M) per study protocol, despite adequate bleed control and no safety concerns.

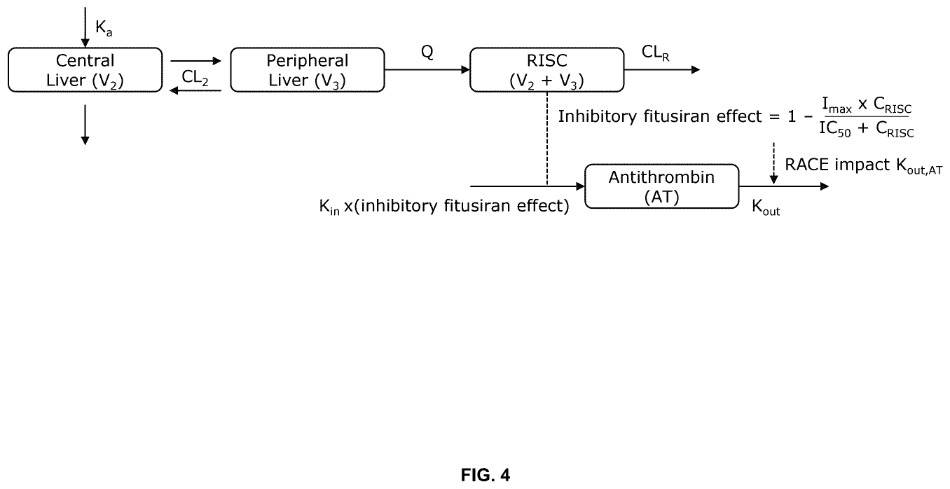

Simulations were used to rationalize the introduction of an additional lower dose of 10 mg (Q2M and monthly QM), to allow more patients to remain on fitusiran. A population semi-mechanistic model, based on the assumption of AT lowering, confirmed the adequacy of the AT-DR, and generated simulations of participant distribution across AT-DR doses that were comparable to observed data (Table 16A). The model was developed using data from all Phase 3 clinical studies and verified using observed data from the Phase 3 ATLAS-OLE open-label extension study. The model (FIG. 4) considered clearance (CL₂) between the central liver (V₂; volume of distribution of central liver compartment) and peripheral liver (V₃; volume of distribution of peripheral liver), the uptake rate constant to the liver (Kₐ), turnover rate of fitusiran from the peripheral liver compartment into the RNA-induced silencing complex (RISC) (Q), and clearance from the RISC compartment (CL_{R}). The inhibitory effect of fitusiran was calculated as provided in FIG. 4 and in Equation 1 below, wherein Imax is the maximum inhibitory effect of fitusiran on Kin (zero-order synthesis rate of antithrombin); Kₐ, uptake rate constant to liver; C_{RISC} is the RISC concentration of active small interfering RNA; and IC₅₀ is the concentration required to produce half-maximal effect of fitusiran.

$$Inhibitory fitusiran effect$$

$$Inhibitory fitusiran effect = 1 - \frac{{I}_{max} \times {C}_{RISC}}{{IC}_{50} + {C}_{RISC}} \qquad Equation 1$$

The inhibitory effect of fitusiran and RACE impact (K_{out,AT}) was considered with the zero-order synthesis rate of antithrombin (Kin) to generate a first-order degradation rate constant for antithrombin (Kₒᵤₜ) (FIG. 4). This modeling was used to identify an additional lower dose that would increase the proportion of patients within the target AT activity range.

TABLE 16A
Simulations identifying participant distribution within
the 15-35% AT activity range across dose regimens

| Dose Regimen | 50 mg | 50 mg | 20 mg | 20 mg | Discontinued |
|---|---|---|---|---|---|
| Population | Q2M | QM | Q2M | QM | (AT <15%) |
| Observed distribution | 40.6% | 15.9% | 29.7% | 2.1% | 11.7% |
| of participants | (115/283) | (45/283) | (84/283) | (6/283) | (33/283) |
| Simulated distribution | 50.59% | 15.83% | 24.15% | 2.72% | 6.7% |
| of participants | (687/1358) | (215/1358) | (328/1358) | (37/1358) | (91/1358) |

Simulations comparing fitusiran 10 mg Q2M with 15 mg QM demonstrated that 0.96% vs 2.8% of simulated participants (N=1358), respectively, had AT levels <15% (Table 16B). As fewer virtual participants had AT levels <15% with fitusiran 10 mg Q2M, the 10 mg dose was expected to help more patients maintain 15-35% AT activity and remain on fitusiran.

TABLE 16B
Simulations predicting the AT activity range
of participants who switched to a lower dose
following AT <15% with fitusiran 20 mg Q2M

| | |
|---|---|
| AT activity | Percent of total virtual patients (N = 1358) |

| range | 10 mg Q2M | 15 mg QM |
|---|---|---|
| AT <15% | 0.96% | 2.8% |
| AT >35% | 0.22% | 0% |
| AT 15-35% | 4.49% | 2.87% |

Further simulations identifying participant distribution within the target AT activity range across dose regimens identified that the addition of fitusiran 10 mg Q2M and QM resulted in only 0.66% of simulated participants discontinuing fitusiran (Table 17), as compared to the observed data (11.7% discontinuing fitusiran on 20 mg Q2M).

TABLE 17
Simulations identifying participant distribution within the 15-35% AT activity
range across dose regimens with the inclusion of 10 mg dose regimens

| | Dose Regimen Population | | | | | | |
|---|---|---|---|---|---|---|---|
| | 50 mg | 50 mg | 20 mg | 20 mg | 10 mg | 10 mg | Discontinued |
| | Q2M | QM | Q2M | QM | Q2M | QM | (AT <15%) |
| Simulated | 50.59% | 15.83% | 24.15% | 2.72% | 4.49% | 1.55 | 0.66 |
| distribution of | (687/ | (215/ | (328/ | (37/ | (61/ | (21/ | (9/ |
| participants | 1358) | 1358) | 1358) | 1358) | 1358) | 1358) | 1358) |

### Simulated Virtual Patient Responses for 10 mg Q2M, 15 mg Q2M, and 10 mg QM De-Escalated Doses

Responses were also simulated for virtual patients having AT activity <15% under a 20 mg Q2M dosing regimen, with 10 mg Q2M, 15 mg Q2M, and 10 mg QM investigated as possible de-escalated doses. Under these different dose de-escalation regimens, the percentages of steady state AT activity were categorized as >35%, 15-35%, and <15, and summarized based on the following two AT measurement scenarios: (1) assuming that all AT values are measurable (as per the current strategy); (2) assuming that only predose AT values are measurable.

Table 9A and Table 9B depict a summary of the simulated proportion of virtual patients with steady state AT activity at 10 mg Q2M, 15 mg Q2M, and 10 mg QM, under scenario (1) assuming that all AT values are measured monthly. Table 9A depicts the simulation with uncertainty. Table 9B depicts the simulation with no uncertainty.

TABLE 9A
Simulated proportion of patients with steady state AT activity assuming
that all AT values are measured monthly, with uncertainty.

| DOSE | <15% (%) | >35% (%) | 15-35% (%) |
|---|---|---|---|
| 10 mg Q2M | 12.5 | 62.5 | 25 |
| | (5/40) | (25/40) | (10/40) |
| 15 mg Q2M | 20 | 57.5 | 22.5 |
| | (8/40) | (23/40) | (9/40) |
| 10 mg QM | 22.5 | 25 | 52.5 |
| | (9/40) | (10/40) | (21/40) |

TABLE 9B
Simulated proportion of patients with steady state AT activity assuming
that all AT values are measured monthly, with no uncertainty.

| DOSE | <15% (%) | >35% (%) | 15-35% (%) |
|---|---|---|---|
| 10 mg Q2M | 23.29 | 1.37 | 75.34 |
| | (17/73) | (1/73) | (55/73) |
| 15 mg Q2M | 58.9 | 0 | 41.1 |
| | (43/73) | (0/73) | (30/73) |
| 10 mg QM | 78.08 | 0 | 21.92 |
| | (57/73) | (0/73) | (16/73) |

Table 10A and Table 10B depict a summary of the simulated proportion of virtual patients with steady state AT activity at 10 mg Q2M, 15 mg Q2M, and 10 mg QM, under scenario (2) assuming that only predose AT values are measured. Table 10A depicts the simulation with uncertainty. Table 10B depicts the simulation with no uncertainty.

TABLE 10A
Simulated proportion of patients with steady state AT activity assuming
that only predose AT values are measured, with uncertainty.

| DOSE | <15% (%) | >35% (%) | 15-35% (%) |
|---|---|---|---|
| 10 mg Q2M | 0 | 100 | 0 |
| | (0/3) | (3/3) | (0/3) |
| 15 mg Q2M | 0 | 100 | 0 |
| | (0/3) | (3/3) | (0/3) |
| 10 mg QM | 0 | 66.67 | 33.33 |
| | (0/3) | (2/3) | (1/3) |

TABLE 10B
Simulated proportion of patients with steady state AT activity assuming
that only predose AT values are measured, with no uncertainty.

| DOSE | <15% (%) | >35% (%) | 15-35% (%) |
|---|---|---|---|
| 10 mg Q2M | 26.67 | 0 | 73.33 |
| | (8/30) | (0/30) | (22/30) |
| 15 mg Q2M | 50 | 0 | 50 |
| | (15/30) | (0/30) | (15/30) |
| 10 mg QM | 100 | 0 | 0 |
| | (30/30) | (0/30) | (0/30) |

Viability of dose de-escalation from 20 mg Q2M to 7.5 mg Q2M

De-escalation scenarios were compared to assess the balance of treatment effect and discontinuation numbers at different doses. 20 mg Q2M, 10 mg Q2M, 10 mg QM, 7.5 mg Q2M, and 7.5 mg QM were compared in terms of practicality of dosing, number of subjects discontinuing treatment, and number of subjects within the therapeutic window. Table 11 depicts the percentages of predicted patients between % AT levels at different doses, and the percentages of predicted patients discontinuing after their last dose.

TABLE 11
Viability of different dose de-escalation regimens

| | | 20 mg Q2M de- | 10 mg Q2M de- | % discontinuing |
|---|---|---|---|---|
| Data | (N) | escalation dose | escalation dose | after last dose |
| Percentage of | 1319 | 17.1% | 5.5% | 4.5% |
| predicted patients | | (225/1319) | (73/1319) | (59/1319) |
| between 15-35% AT | | | | |
| levels* | | | | |

| | | 20 mg Q2M de- | 10 mg QM de- | % discontinuing |
|---|---|---|---|---|
| Data | (N) | escalation dose | escalation dose | after last dose |
| Percentage of | 1319 | 17.1% | 0.6% | 9.4% |
| predicted patients | | (225/1319) | (8/1319) | (124/1319) |
| between 15-35% AT | | | | |
| levels* | | | | |

| | | 20 mg Q2M de- | 7.5 mg Q2M de- | % discontinuing |
|---|---|---|---|---|
| Data | (N) | escalation dose | escalation dose | after last dose |
| Percentage of | 1319 | 17.1% | 6.1% | 3.9% |
| predicted patients | | (225/1319) | (80/1319) | (52/1319) |
| between 15-35% AT | | | | |
| levels* | | | | |

| | | 20 mg Q2M de- | 7.5 mg QM de- | % discontinuing |
|---|---|---|---|---|
| Data | (N) | escalation dose | escalation dose | after last dose |
| Percentage of | 1319 | 17.1% | 1.9% | 8.1% |
| predicted patients | | (225/1319) | (25/1319) | (107/1319) |
| between 15-35% AT | | | | |
| levels* | | | | |

Of all the dosing scenarios, 7.5 mg Q2M theoretically provided the maximum benefit for retaining patients in the therapeutic window compared to those discontinuing treatment. From a practical point of view, however, the 10 mg Q2M dose is easier to administer and was the next best option after 7.5 mg Q2M. Thus, the 10 mg Q2M dose was selected as the preferred dose de-escalation option.

### Example 3: Fitusiran Drug Product

A new Fitusiran drug product (Fitusiran) was developed for subcutaneous administration. The drug product is an aqueous, clear, sterile and non-pyrogenic solution for injection in a single use container which contains 40 mg fitusiran drug substance per mL. The drug product is supplied in a prefilled pen (PFP) with a nominal fill volume of 0.5 mL drug product solution. The new 40 mg/mL drug product formulation contains the active ingredient fitusiran at a concentration of 40 mg/mL (equivalent to 42.4 mg fitusiran sodium per mL) and is formulated in a 5 mM PBS solution.

Fitusiran drug substance (DS) is a small interfering ribonucleic acid (siRNA) and consists of chemically synthesized double stranded oligonucleotides covalently linked to a ligand containing three GalNAc (N-acetyl galactose amine) residues. The DS is provided as a lyophilized powder. Due to its large molecular size (16.2 kDA), fitusiran DS is unable to be sufficiently absorbed when administered orally. Therefore, the DP formulation was developed as an aqueous, sterile, clear solution, administered subcutaneously, for single use.

TABLE 12
Abbreviations of nucleotide monomers used in nucleic
acid sequence representation. It will be understood
that these monomers, when present in an oligonucleotide,
are mutually linked by 5′-3′-phosphodiester bonds.

| Abbreviation | Nucleotide (s) |
|---|---|
| A | Adenosine-3′-phosphate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide (G, A, C, T or U) |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol |
| | Hyp-(GalNAc-alkyl)3 |

The chemical structure of Fitusiran, shown in FIG. 1 (see also Table 12 above), is represented using an expanded structural formula showing the phosphate backbone. The bases involved in base pair formation are connected with a dotted line. The structure of L96, the GalNAc containing ligand, and the linker conjugating the ligand to the 3′-end of the sense strand is also presented below. The molecular formulas and masses of the duplex and the two single strands (AD-116858, sense strand; A-116861, antisense strand) of the Fitusiran duplex (AD-57213) are also provided in FIG. 1.

### Example 4: Fitusiran Formulation Development

The fitusiran formulation was designed for subcutaneous administration. Formulations designed for subcutaneous administration should not be too acidic or too alkaline to avoid the risk of increased irritation and chemical incompatibility. With due consideration of tonicity, pH, and viscosity, the formulation was designed to be as close to physiological as possible. To maintain the isotonicity and buffer capacity of the drug product, the drug substance was dissolved in a 5 mM phosphate buffered saline (PBS).

The formulation development addressed the intended quality target product profile (QTPP). The drug product (DP) was targeted to be a sterile, clear solution for injection containing 40 mg/mL drug substance (DS). The drug product is supplied in a single-use prefilled pen (PFP) containing a 20 mg dose (in 0.5 mL) for parenteral administration and is indicated for patients with hemophilia A or B with or without inhibitors against recombinant coagulation factors for routine prophylaxis to prevent or reduce the frequency of bleeding episodes. Efficacy and safety are to be ensured through shelf-life, which is not less than 36 months at launch. Storage instructions include storing between +2° C. and +8° C. protected from light. Fitusiran drug product may be stored at room temperature, not to exceed 30° C. for a single period of up to 3 months within the expiration date printed on the label. After storage at room temperature, the product is not returned to the refrigerator.

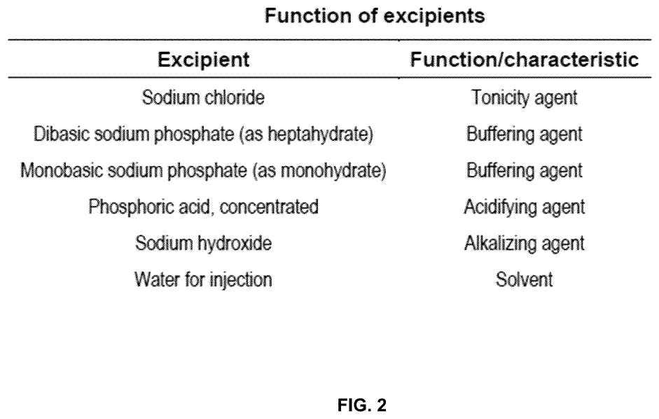

The excipients used in the manufacture of fitusiran DP were selected based on their characteristics and functionalities and the requirements of the QTPP. The excipients included in the product composition are listed together with their functionality in FIG. 2. All excipients are well known for parenteral products and listed in Ph. Eur. and/or USP/NF (Ph.Eur.=European Pharmacopeia; USP=United States Pharmacopeia). The excipients are freely mixable or sufficiently soluble in water and well tolerated pharmacopoeial standard excipients for parenteral solutions.

In the new 40 mg/mL formulation, the DS concentration was decreased to 40 mg/mL and the pH of the PBS had to be adapted. An initial experiment to select a suitable PBS buffer containing the isotonicity agent sodium chloride with a final osmolality of ~253 mOsmol/kg was performed. Previous experiments showed that osmolality of 40 mg/mL DS dissolved in water is ~47 mOsmol/kg. Thus, an osmolality close to 300 mOsmol/kg was the goal for the dissolution of fitusiran DS. Preliminary studies showed that fitusiran DS dissolved at a concentration of 40 mg/mL in PBS solution pH 7.2, resulted in pH 7.038, the target pH of the drug product solution (7.0±0.2). For these reasons, the PBS solution pH 7.2 was selected to be used as compounding buffer for fitusiran DP. pH and density values were measured at room temperature and +20° C. and +25° C., respectively.

After selecting the PBS buffer with the suitable pH for dissolving fitusiran DS to reach a final pH of 7.0 of the DP solution, the compounding process had to be reproduced at laboratory scale. In this context, the ultraviolet (UV)-assay was established to determine the DS concentration at the 80% filling volume step and to calculate the final batch size. Additionally, the density values were measured at +20° C. and +25° C. while viscosity was measured in the temperature range between +5° C. and +30° C. Analytical analyses (e.g., density, viscosity, etc.) are further described in Example 5.

The PBS solution is composed of sodium chloride, dibasic sodium phosphate (heptahydrate), and monobasic sodium phosphate (monohydrate). Sodium hydroxide and diluted phosphoric acid are used to adjust the PBS pH to pH=7.2. Preliminary studies were performed preparing PBS at pH=7.2 with different sodium chloride amounts. The buffer composition of the selected 5 mM PBS is reported in Table 13.

TABLE 13
Composition of 1000 g of 5 mM phosphate buffered saline (PBS)

| Ingredient | Target weight | |
|---|---|---|
| Mon basic sodium phosphate (monohydrate)^{a} | 0.209 | g |
| Dibasic sodium phosphate (heptahydrate)^{b} | 0.929 | g |
| Sodium chloride^{c} | 7.64 | g |

| | |
|---|---|
| Sodium hydroxide 0.1M | q.s. |
| Phosphoric acid 0.1M | q.s. |
| Purified water | Filled up to final q.s. |

^{a}Sodium dihydrogen phosphate monohydrate
^{b}Di-sodium hydrogen phosphate heptahydrate
^{c}Low in endotoxins
q.s. = quantity sufficient

Fitusiran drug product manufacturing consisted of dissolving the required amount of the powdered (lyophilized) fitusiran drug substance in 5 mM phosphate buffered saline and adjusting the pH with sodium hydroxide or phosphoric acid to approximately 7.0, followed by sterile filtration and filling.

Once the PBS buffer formulation was established, the DP compounding process was mimicked. The amount of DS needed was calculated considering water content and non-denaturing IPRP (ion pair reversed-phase) purity. It was then dissolved in 80% of the required volume of buffer. UV assay and pH measurements were performed at this step. The amount of additional buffer required to adjust to 40 mg/mL was then calculated based on the UV results and used for dilution of the concentrated formulation.

The final composition of the DP is shown in Table 14 and a comparison of the composition of the 40 mg/mL and 100 mg/mL DP is shown in Table 1B above. The fitusiran drug product formulation has the following physicochemical properties: pH of about 6.8 to about 7.2; Osmolality of about 300 mOsm/kg; and a density of about 1.02364 g/mL.

TABLE 14
Final composition of fitusiran DP (40 mg/mL)

| Ingredient | Concentration | |
|---|---|---|
| Fitusiran (free acid) | 40 | mg/mL |
| Monobasic sodium phosphate (monohydrate) ^{a} | 0.2095 | mg/mL |
| Dibasic sodium phosphate (heptahydrate) ^{b} | 0.933 | mg/mL |
| Sodium chloride ^{c} | 7.672 | mg/mL |

| | |
|---|---|
| Purified water | Ad 1 mL |
| Phosphoric acid 0.1M | For pH adjustment |
| Sodium hydroxide 0.1M | For pH adjustment |

^{a} Sodium dihydrogen phosphate monohydrate
^{b} Di-sodium hydrogen phosphate heptahydrate
^{c} Low in endotoxins

### Example 5: Analytical Evaluations of Fitusiran Drug Product

Various analytical evaluations of the 40 mg/mL Fitusiran drug product were performed to demonstrate that the drug product was physically and chemically stable. The physicochemical properties of the final DP are also reported below.

### Appearance

The visual appearance of the formulation is clear and colorless to pale yellow according to its specification. The solution is essentially free of visible particulates.

### Density

The density values at +20° C. and at +25° C. are reported in Table 15. Density was measured with DMA 4500M density instrument from Anton Paar (H770, Frankfurt). The density of the drug product solution will be determined for every batch during manufacturing and used for calculations of the final batch size.

TABLE 15
Density values of the DP

| Density | pH 7.2 |
|---|---|
| Density [g/cm³] at +20° C. | 1.0232 |
| Density [g/cm³] at +25° C. | 1.0219 |

### Tonicity

The tonicity of fitusiran solution for injection 40 mg/mL was adjusted with sodium chloride to a target of 300 mOsmol/kg. This is suitable for subcutaneous injection.

### Viscosity

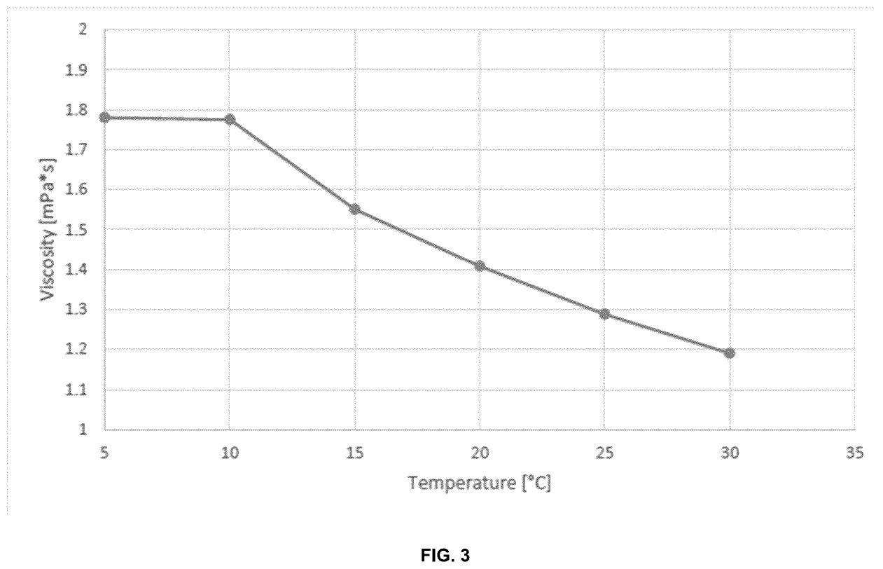

The viscosity of fitusiran drug product solution for injection 40 mg/mL was determined at different temperatures. It was shown that the viscosity of the DP is temperature dependent and decreases with increasing temperatures. FIG. 3 shows the viscosity in correlation to the temperature.

### Assay of Fitusiran Drug Product by UV

UV absorbance method was used for the determination of assay (mg/mL) of fitusiran in the fitusiran drug product. The absorbance of a suitably diluted drug product in 0.9% saline is measured with a UV spectrophotometer at 260 nm.

$$C = ( A \times F \times M ) / ( ε \times b ) ,$$

where A is the measured absorbance, F is the dilution factor, b is the path length of the cell (1 cm), ε is molar absorptivity of duplex reference standard, M is the molecular weight and C is the concentration (mg/mL). To account for duplex purity, the fitusiran assay result for a given drug product is corrected for its purity factor (multiplied by (non-denaturing IP-RP HPLC area-%)/100).

The Assay of the fitusiran drug product (mg/mL) was determined from UV spectrophotometry and corrected for duplex purity from the non-denaturing IP RP-HPLC method, and the results are reported on the basis of the concentration of the H-form (free acid) of the duplex. The concentration at the 80% step was 50.13 mg/mL and after the filling up to the final volume, the concentration was 40.04 mg/mL. This provided evidence that the compounding process at laboratory scale was successful.

The determination of the UV-assay at the 80% and 100% filling steps as well as the tested physicochemical properties demonstrated the successful development of the new formulation (40 mg/mL).

### pH of Fitusiran Drug Product

To achieve a pH closely to the physiological pH of 7.4, fitusiran drug product solution for injection 40 mg/mL is buffered to a target of pH 7.0. This pH enhances the tolerability and ensures a stable DP throughout shelf life.

### Osmolality of Fitusiran Drug Product

The osmolality of fitusiran drug product is based on principle of freezing-point depression. Osmolality was reported as mOsmol/kg value. Osmolality was measured with Osmomat010 Fa. Gonotec. The osmolality of DP 40 mg/mL was 303 [mOsmol/kg].

### Example 6: Container Closure System of Fitusiran Drug Product

The prefilled pen (PFP) presentation consists of a device constituent part, which integrates and encloses a primary container (referred to as a bulk prefilled syringe [PFS]) with a nominal fill volume of 0.5 mL. This single-use presentation delivers a 20 mg dose (0.5 mL of 40 mg/mL fitusiran solution) per injection. Extractable volume is not less than 0.5 mL.

The bulk prefilled syringe is the primary container closure system. The 1 mL long glass syringe is equipped with a staked needle covered with a rigid needle shield and closed with a plunger stopper.

### Example 7: Medication Guide for Fitusiran Drug Product

Important Information about Fitusiran Drug Product

Fitusiran helps your blood form clots. Do not stop using fitusiran drug product without talking to your healthcare provider. If you miss doses or stop using fitusiran drug product, you may no longer be protected against bleeding. Use of a clotting factor concentrate (CFC) or bypassing agent (BPA) to help protect against bleeding must be stopped within 7 days after your first dose of fitusiran drug product.

Your healthcare provider may prescribe on demand CFC (factor VIII or factor IX products) or BPA if you bleed during treatment with fitusiran drug product. Carefully follow your healthcare provider's instructions regarding when to use on-demand treatment with CFC or BPA, including the prescribed dose and timing of the CFC or BPA.

Possible Side Effects with Fitusiran Drug Product

### Abnormal Blood Clotting (Thrombotic Events).

Serious blood clots have happened in people treated with fitusiran drug product. Fitusiran drug product can cause blood clots to form in blood vessels in your arms, legs, lungs, heart, brain, eyes, or head. Your risk of blood clots is greater if your antithrombin (AT) blood level is persistently less than 15% or if you have certain other conditions. Get medical help right away if you get any of these signs or symptoms of blood clots during or after treatment with fitusiran drug product:
Swelling, pain or redness in arms or legs
Coughing up blood
Shortness of breath
Severe chest pain or tightness of the chest
Fast heart rate
Feeling faint or passing out
Severe or persistent headache
Difficulty speaking or understanding language
Feel confused
Numbness of weakness in your face, arms or legs
Sudden loss or changes in your vision, eye pain, or swelling

### Gallbladder Disease

Fitusiran drug product can cause gallstones (cholelithiasis) and inflammation of your gallbladder (cholecystitis), which might require surgery to remove your gallbladder. Tell your healthcare provider right away if you develop stomach (abdomen) pain, indigestion, nausea or vomiting. Your healthcare provider may temporarily or permanently stop your treatment with fitusiran drug product if you develop any of these symptoms.

### Liver Problems

Fitusiran drug product can cause an increase in your blood liver enzymes. Your healthcare provider will do blood tests to check your liver function before and during treatment with fitusiran drug product.

### What is Fitusiran Drug Product?

Fitusiran drug product is a prescription medicine used for routine prophylaxis to prevent or reduce the frequency of bleeding episodes in adults and children 12 years and older with hemophilia A or B with or without factor VIII or IX inhibitors.

It is not known if fitusiran drug product is safe and effective in children younger than 12 years of age.

Before receiving fitusiran drug product, tell your healthcare provider about all of your medical conditions, including if you:
have liver problems
have a history of gallbladder disease
are pregnant or plan to become pregnant. It is not known if fitusiran drug product may harm your unborn baby.
Females who are able to become pregnant: Hormonal birth control (contraception) may increase your risk of blood clots if used during treatment with fitusiran drug product. If you use hormonal birth control (contraception), talk to your healthcare provider about effective forms of non-hormonal birth control (contraception) options you can use before starting and during treatment with fitusiran drug product.
are breastfeeding or plan to breastfeed. It is not known if fitusiran drug product passes into your breast milk.

Tell your healthcare provider about all the medicines you take, including prescription and over-the-counter medicines, vitamins, and herbal supplements. Keep a list of them to show your healthcare provider and pharmacist when you get a new medicine.

How should I Use Fitusiran Drug Product?

See the detailed “Instructions for Use” that comes with your fitusiran drug product for information on how to prepare and inject a dose of fitusiran drug product, and how to properly throw away (dispose of) the used prefilled pen, vial, needle and syringe.

Use fitusiran drug product exactly as prescribed by your healthcare provider.

Your healthcare provider may change your dose or how often you inject fitusiran drug product based on your AT blood level to help reduce your chance of abnormal blood clots. Your healthcare provider will check your AT blood level:
before starting fitusiran drug product and at month 1, 3, 5 and 6 during fitusiran drug product treatment.
after any change in your dose of fitusiran drug product.
at yearly routine visits after your healthcare provider determines your target dose.

It is recommended that fitusiran drug product be given by or under the supervision of an adult in children 12 to 17 years of age.

Fitusiran drug product is given as an injection under your skin (subcutaneous) by you or a caregiver.

Your healthcare provider should show you or your caregiver how to prepare, measure, and inject your dose of fitusiran drug product before you inject yourself for the first time.

Do not attempt to inject yourself or another person unless you have been taught how to do so by a healthcare provider.

After your first dose of fitusiran drug product, your healthcare provider will let you know when to take your next dose and how much fitusiran drug product to inject.

If you miss a dose of fitusiran drug product on your scheduled day, you should give the dose as soon as possible. After that, resume your dosing schedule either 1 time every month or 1 time every two months from the last dose, as instructed by your healthcare provider.

### What are the Possible Side Effects of Fitusiran Drug Product?

The most common side effects of fitusiran drug product include:
viral infection
common cold symptoms
bacterial infection

These are not all of the possible side effects of fitusiran drug product.

Call your doctor for medical advice about side effects. You may report side effects to the FDA at 1-800-FDA-1088.

How should I Store Fitusiran Drug Product?

### 50 mg Prefilled Pen

Store fitusiran drug product prefilled pen in the refrigerator between 36° F. to 46° F. (2° C. to 8° C.) in the original carton to protect from light.

Fitusiran drug product prefilled pen may be stored at room temperature between 59° F. to 86° F. (15° C. to 30° C.) for a single period of up to 3 months within the expiration date printed on the label.

Throw away (dispose of) the fitusiran drug product prefilled pen after 3 months at room temperature or at the expiration date, whichever comes first.

Do not return fitusiran drug product prefilled pen to the refrigerator after storing at room temperature.

### 20 mg Prefilled Pen

Store fitusiran drug product prefilled pen in the refrigerator between 36° F. to 46° F. (2° C. to 8° C.) in the original carton to protect from light.

Fitusiran drug product prefilled pen may be stored at room temperature between 59° F. to 86° F. (15° C. to 30° C.) for a single period of up to 3 months within the expiration date printed on the label.

Throw away (dispose of) the fitusiran drug product prefilled pen after 3 months at room temperature or at the expiration date, whichever comes first.

Do not return fitusiran drug product prefilled pen to the refrigerator after storing at room temperature.

### 20 mg Vial

Store fitusiran drug product vial in the refrigerator between 36° F. to 46° F. (2° C. to 8° C.) or at room temperature between 59° F. to 86° F. (15° C. to 30° C.) in the original carton to protect from light.

Do not return fitusiran drug product vial to the refrigerator after storing at room temperature.

Do not shake the fitusiran drug product vial or prefilled pen at any time.

Do not heat the fitusiran drug product vial or prefilled pen.

Do not freeze the fitusiran drug product vial or prefilled pen.

Do not put the fitusiran drug product vial or prefilled pen into direct sunlight.

Throw away (dispose of) any unused fitusiran drug product.

Keep fitusiran drug product and all medicines out of the reach of children.

General Information about the Safe and Effective Use of Fitusiran Drug Product

Medicines are sometimes prescribed for purposes other than those listed in a Medication Guide. Do not use fitusiran drug product for a condition for which it was not prescribed. Do not give fitusiran drug product to other people even if they have the same symptoms you have. It may harm them. You can ask your healthcare provider or pharmacist for information about fitusiran drug product that is written for health professionals.

### What are the Ingredients in Fitusiran Drug Product?

### Active Ingredient: Fitusiran

Inactive ingredients: dibasic sodium phosphate, monobasic sodium phosphate, sodium chloride, and Water for Injection, USP. Phosphoric acid (concentrated) and sodium hydroxide may be added to adjust pH to 7.

### Example 8: Instructions for Fitusiran Drug Product Use from a 20 mg/0.2 mL Single-Dose Vial

This Instructions for Use contains information on how to inject fitusiran drug product from a vial.

Read this Instructions for Use before using fitusiran drug product. Do not inject yourself or someone else until you have been shown how to inject fitusiran drug product from a vial. Your healthcare provider can show you or your caregiver how to prepare and inject a dose of fitusiran drug product from a vial before you do it yourself for the first time. Keep this Instructions for Use. Call your healthcare provider if you have any questions.

Fitusiran drug product is only for use in adults and children 12 years of age and older. In children 12 to 17 years of age it is recommended that fitusiran drug product be given by or under the supervision of an adult.

This fitusiran drug product vial is for single-use only. It contains 20 mg of fitusiran for injection under the skin (subcutaneous injection).

### Important Information to Know Before Injecting Fitusiran Drug Product

Read all of the instructions carefully before using fitusiran drug product.

Ask your healthcare provider how often you will need to inject the medicine.

Check your prescription label to make sure you draw up the correct medicine dose prescribed by your healthcare provider.

Throw away (dispose of) the used vial, syringe and needle right away after use.

Only use the vial one time. After you inject your dose, throw away (dispose of) any unused fitusiran drug product left in the vial. Do not save any unused fitusiran drug product in the vial for later use.

fitusiran drug product is for subcutaneous use only. Do not inject fitusiran drug product into a vein.

### Storing Fitusiran Drug Product Vial

Store fitusiran drug product vial either in the refrigerator between 36° F. to 46° F. (2° C. to 8° C.) or at room temperature between 59° F. to 86° F. (15° C. to 30° C.) in the original carton to protect from light.

Do not return fitusiran drug product vial to the refrigerator after storing at room temperature.

Do not shake the fitusiran drug product vial at any time.

Do not heat the fitusiran drug product vial.

Do not freeze the fitusiran drug product vial.

Do not put the fitusiran drug product vial into direct sunlight.

Keep fitusiran drug product vial and all medicines out of the reach of children.

### A. Get Ready to Inject

### A1) Gather the Supplies

Find a clean, flat work surface. Make sure you have the following supplies: fitusiran drug product 20 mg Vial, 1 mL syringe, injection needle (27-gauge×0.5 inches), sharps container, 2 alcohol wipes, cotton ball or gauze. Note: the syringe and needle should have Luer Lock connections.

### A2) Check the Vial

Do not use the fitusiran drug product vial if it has been dropped or damaged.

Check the label on the vial to be sure that you have the correct prescribed medicine and dose.

Check expiration date (EXP:).

Do not use fitusiran drug product if the expiration date on the vial has passed.

### A3) Check the Medicine

Look at the medicine in the vial to make sure it is clear and colorless to pale yellow.

Do not use fitusiran drug product if the liquid in the vial is discolored or cloudy, or if it contains visible flakes or particles.

### A4) if the Fitusiran Drug Product Vial was Stored in the Refrigerator, Warm Up for 30 Minutes.

Set the fitusiran drug product vial on a clean, flat surface and let it warm up at room temperature between 59° F. to 86° F. (15° C. to 30° C.) for at least 30 minutes.

Do not heat the fitusiran drug product vial or put in direct sunlight.

A5) Wash Your Hands Well with Soap and Water.
A6) Remove the Protective Cap from the Fitusiran Drug Product Vial.

Do not remove the stopper.

A7) Wipe the Rubber Stopper of the Vial with an Alcohol Wipe and Allow it to Dry.

### B. Choose and Prepare the Injection Site

### B1) Choose the Injection Site.

### Thigh

Stomach except for the 2 inches (5 cm) around your belly button (navel).

A caregiver can also inject in the outer area of your upper arm.

Do not inject into skin that is tender, damaged, has bruises or scars, or into areas with visible veins.

Do not inject into a vein. For subcutaneous use only.

Do not inject through clothes.

### B2) Prepare the Injection Site.

Clean the injection site with an alcohol wipe.

Let the skin dry before injecting.

Do not touch the injection site again or blow on it before the injection.

### C. Prepare the Injection

Contact your healthcare provider if you have any questions about preparing your dose.

### C1) Attach the Needle to the Syringe and Remove the Needle Cap.

Attach the needle to the syringe.

Hold the syringe body and pull the needle cap straight off the syringe.

Do not touch the needle.

Do not recap the needle.

### C2) Withdraw the Solution.

Withdraw slightly more than your prescribed dose into the syringe.

Push the needle through the center of the rubber vial stopper.

Leave the needle in the vial and turn both the syringe and vial upside down (vial on top).

Hold the syringe and vial firmly in one hand. Make sure the tip of the needle is in the fitusiran drug product solution.

With your other hand, pull back on the plunger rod to withdraw slightly more than your prescribed dose into the syringe.

Withdraw slightly more than 0.2 mL for a 20 mg dose.

Withdraw slightly more than 0.1 mL for a 10 mg dose.

### C3) Check for Air Bubbles.

Keep the needle in the vial and make sure both syringe and vial are upside down (vial on top).

If air bubbles are in the syringe, hold the syringe straight up and tap the side of the syringe until the bubbles float to the top.

Push the plunger rod up to push the bubbles out of the syringe.

### C4) Adjust Plunger to Your Prescribed Dose.

Push the plunger rod to push solution back into the vial until you have the prescribed dose in the syringe.

Push plunger rod to 0.2 mL for a 20 mg dose.

Push plunger rod to 0.1 mL for a 10 mg dose.

If the medicine in the syringe is less than your prescribed dose, repeat steps C2, C3 and C4 until you have your correct dose.

Remove the needle from the vial. Do not let the needle touch anything.

### D. Inject Fitusiran Drug Product

### D1) Pinch a Fold of Skin and Insert the Needle.

Gently pinch a fold of skin at the cleaned injection site. Insert the needle into the skin fold at a 450 to 900 angle.

### D2) Inject Fitusiran Drug Product.

Relax the skin pinch. Slowly push the plunger rod all the way to the bottom of the syringe body and inject all of the fitusiran drug product solution.

D3) Gently Pull Out the Needle and Syringe from the Injection Site.

Do not recap the used needle to avoid a needle-stick injury.

Do not rub the skin after the injection.

If you see any blood at the site, lightly dab it with a cotton ball or gauze pad. If your injection site becomes red or sore, call your healthcare provider right away.

### E. Throw Away (Dispose of) Used Vial, Needle and Syringe

E1) Throw Away (Dispose of) Your Used Vial, Needle and Syringe in an FDA-Cleared Sharps Disposal Container Right Away after Use.

Do not re-use the vial, needle, or syringe.

Keep your sharps disposal container out of the reach of children

If you do not have an FDA-cleared sharps disposal container, you may use a household container that is:
made of a heavy-duty plastic,
can be closed with a tightfitting, puncture-resistant lid, without sharps being able to come out,
upright and stable during use,
leak-resistant, and
properly labeled to warn of hazardous waste inside the container.

When your sharps disposal container is almost full, you will need to follow your community guidelines for the right way to dispose of your sharps disposal container. There may be state or local laws about how you should throw away used fitusiran drug product needles and syringes.

Do not dispose of your used sharps disposal container in your household trash unless your community guidelines permit this.

Do not recycle your used sharps disposal container.

### Example 9: Instructions for Fitusiran Drug Product Use, 50 mg/0.5 mL Single-Dose Prefilled Pen

This Instructions for Use contains information on how to inject fitusiran drug product.

Read this Instructions for Use before using the fitusiran drug product Prefilled Pen. Do not inject yourself or someone else until you have been shown how to inject fitusiran drug product Prefilled Pen. Your healthcare provider can show you or your caregiver how to prepare and inject a dose of fitusiran drug product before you try to do it yourself for the first time. Keep this Instructions for Use. Call your healthcare provider if you have any questions.

Fitusiran drug product Prefilled Pen is only for use in adults and children 12 years of age and older. It is recommended that fitusiran drug product Prefilled Pen be given by adults or under adult supervision to children 12 to 17 years of age.

This fitusiran drug product Prefilled Pen is a single-dose device. It contains 50 mg of fitusiran for injection under the skin (subcutaneous injection).

### Important Information to Know Before Injecting Fitusiran Drug Product

Read all of the instructions carefully before using the fitusiran drug product Prefilled Pen.

Ask your healthcare provider how often you will need to inject the medicine.

Throw away (dispose of) the used fitusiran drug product Prefilled Pen right away after use.

Do not re-use a fitusiran drug product Prefilled Pen.

Fitusiran drug product Prefilled Pen is for subcutaneous use only. Do not inject fitusiran drug product Prefilled Pen into a vein.

### Storing Fitusiran Drug Product Prefilled Pen

Store fitusiran drug product Prefilled Pen in the refrigerator between 36° F. to 46° F. (2° C. to 8° C.) in the original carton to protect from light.

Fitusiran drug product Prefilled Pen may be stored at room temperature between 59° F. to 86° F. (15° C. to 30° C.) for a single period of up to 3 months within the expiration date printed on the label.

Do not return fitusiran drug product Prefilled Pen to the refrigerator after storing at room temperature.

Do not shake the fitusiran drug product Prefilled Pen at any time.

Do not heat the fitusiran drug product Prefilled Pen.

Do not freeze the fitusiran drug product Prefilled Pen.

Do not put the fitusiran drug product Prefilled Pen into direct sunlight.

Keep fitusiran drug product Prefilled Pen and all medicines out of the reach of children.

### A. Get Ready to Inject

### A1) Gather Supplies

Find a clean, flat work surface. Make sure you have the following supplies: Fitusiran drug product Prefilled Pen, 1 alcohol wipe, 1 cotton ball or gauze, a sharps disposal container.

### A2) Check the Fitusiran Drug Product Prefilled Pen

Note: The plunger stopper in the middle of the Window is normal.

Do not use the fitusiran drug product Prefilled Pen if it has been dropped or damaged.

Do not use the fitusiran drug product Prefilled Pen if the Blue Cap is missing or not securely attached.

Do not use the fitusiran drug product Prefilled Pen if the Window is yellow before use. A yellow Window means the fitusiran drug product Prefilled Pen has been used.

### A3) Look at the Label

Check the label to be sure that you have the correct prescribed medicine and dose.

Check the expiration date.

Do not use the fitusiran drug product Prefilled Pen if the expiration date has passed.

### A4) Check the Medicine

Look at the medicine through the Window, it should be clear and colorless to pale yellow.

Note: You may see air bubbles, this is normal.

Do not use the fitusiran drug product Prefilled Pen if the liquid is discolored or cloudy, or if it contains visible flakes or particles.

### A5) if Fitusiran Drug Product Prefilled Pen was Stored in the Refrigerator, Warm Up for 30 Minutes.

Lay the fitusiran drug product Prefilled Pen on a clean, flat surface and let it warm up at room temperature between 59° F. to 86° F. (15° C. to 30° C.) for at least 30 minutes.

Do not heat the fitusiran drug product Prefilled Pen or put into direct sunlight.

### B. Choose and Prepare Your Injection Site

B1) Wash Your Hands Well with Soap and Water

### B2) Choose an Injection Site

### Thigh

Stomach except for the 2 inches (5 cm) around your belly button (navel).

A caregiver can also inject in the outer area of your upper arm.

Do not inject into skin that is tender, damaged, has bruises or scars, or into areas with visible veins.

For subcutaneous use only. Do not inject into a vein.

Do not inject through clothes.

### B3) Prepare the Injection Site

Clean the injection site with an alcohol wipe.

Let the skin dry before injecting.

Do not touch the injection site again or blow on it before the injection.

### C. Give the Injection

### C1) Remove Blue Cap

Remove the Blue Cap by pulling it straight off as shown. Do not twist the Blue Cap off.

Do not remove the Blue Cap until you are ready to inject.

Do not touch the Orange Needle Cover with your fingers. The Needle is inside.

Do not put the Blue Cap back on the fitusiran drug product Prefilled Pen after you have removed it.

Note: You may see a droplet on the needle tip. This is normal.

### C2) Place Fitusiran Drug Product Prefilled Pen on Injection Site

Hold the fitusiran drug product Prefilled Pen as shown so you can see the Window.

Place the Orange Needle Cover on the skin at a 90-degree angle.

### C3) Press Down Firmly→Watch Window Turn Fully Yellow

Press down and hold the fitusiran drug product Prefilled Pen firmly against the skin until you cannot see the Orange Needle Cover.

There will be a “click” when the injection starts, and

The Window will start to turn yellow.

Keep pressing the fitusiran drug product Prefilled Pen against the skin and watch the Window.

The Window will turn completely yellow, and

You may hear a 2nd “click.” The injection is complete.

### C4) Remove

After you have completed your injection pull straight up to remove fitusiran drug product Prefilled Pen from the skin. The Orange Needle Cover will cover the needle.

If you see any blood at the site, lightly dab a cotton ball or gauze pad.

Do not rub the skin after the injection.

If the Window does not turn completely yellow, or if it looks like you may not have received a full dose, call your healthcare provider right away.

Do not give a second dose without speaking to your healthcare provider.

### D. Throw Away (Dispose of) Used Fitusiran Drug Product Prefilled Pen

Do not put the Blue Cap back on.

Keep your sharps disposal container out of the reach of children.

### How to Dispose of (Throw Away) Fitusiran Drug Product Prefilled Pen

Put your used fitusiran drug product Prefilled Pen and Blue Cap in an FDA-cleared sharps disposal container right away after use.

Do not dispose of (throw away) fitusiran drug product Prefilled Pen and Blue Cap in your household trash.

If you do not have an FDA-cleared sharps disposal container, you may use a household container that is:
made of a heavy-duty plastic,
can be closed with a tight-fitting, puncture-resistant lid, without sharps being able to come out,
upright and stable during use,
leak-resistant, and
properly labeled to warn of hazardous waste inside the container.

When your sharps disposal container is almost full, you will need to follow your community guidelines for the right way to dispose of your sharps disposal container. There may be state or local laws about how you should throw away used fitusiran drug product Prefilled Pens.

Do not dispose of your used sharps disposal container in your household trash unless your community guidelines permit this.

Do not recycle your used sharps disposal container.

### Example 10: Instructions for Fitusiran Drug Product Use, 20 mg/0.5 mL Single-Dose Prefilled Pen

This Instructions for Use contains information on how to inject fitusiran drug product.

Read this Instructions for Use before using the fitusiran drug product Prefilled Pen. Do not inject yourself or someone else until you have been shown how to inject fitusiran drug product Prefilled Pen. Your healthcare provider can show you or your caregiver how to prepare and inject a dose of fitusiran drug product before you try to do it yourself for the first time. Keep this Instructions for Use. Call your healthcare provider if you have any questions.

Fitusiran drug product Prefilled Pen is only for use in adults and children 12 years of age and older. It is recommended that fitusiran drug product Prefilled Pen be given by adults or under adult supervision to children 12 to 17 years of age.

This fitusiran drug product Prefilled Pen is a single-dose device. It contains 20 mg of fitusiran for injection under the skin (subcutaneous injection).

### Important Information to Know Before Injecting Fitusiran Drug Product

Read all of the instructions carefully before using the fitusiran drug product Prefilled Pen.

Ask your healthcare provider how often you will need to inject the medicine.

Throw away (dispose of) the used fitusiran drug product Prefilled Pen right away after use.

Do not re-use a fitusiran drug product Prefilled Pen.

Fitusiran drug product Prefilled Pen is for subcutaneous use only. Do not inject fitusiran drug product Prefilled Pen into a vein.

### Storing Fitusiran Drug Product Prefilled Pen

Store fitusiran drug product Prefilled Pen in the refrigerator between 36° F. to 46° F. (2° C. to 8° C.) in the original carton to protect from light.

Fitusiran drug product Prefilled Pen may be stored at room temperature between 59° F. to 86° F. (15° C. to 30° C.) for a single period of up to 3 months within the expiration date printed on the label.

Do not return fitusiran drug product Prefilled Pen to the refrigerator after storing at room temperature.

Do not shake the fitusiran drug product Prefilled Pen at any time.

Do not heat the fitusiran drug product Prefilled Pen.

Do not freeze the fitusiran drug product Prefilled Pen.

Do not put the fitusiran drug product Prefilled Pen into direct sunlight.

Keep fitusiran drug product Prefilled Pen and all medicines out of the reach of children.

### A. Get Ready to Inject

### A1) Gather Supplies

Find a clean, flat work surface. Make sure you have the following supplies: Fitusiran drug product Prefilled Pen, 1 alcohol wipe, 1 cotton ball or gauze, a sharps disposal container.

### A2) Check the Fitusiran Drug Product Prefilled Pen

Note: The plunger stopper in the middle of the Window is normal.

Do not use the fitusiran drug product Prefilled Pen if it has been dropped or damaged.

Do not use the fitusiran drug product Prefilled Pen if the Blue Cap is missing or not securely attached.

Do not use the fitusiran drug product Prefilled Pen if the Window is yellow before use. A yellow Window means the fitusiran drug product Prefilled Pen has been used.

### A3) Look at the Label

Check the label to be sure that you have the correct prescribed medicine and dose.

Check the expiration date.

Do not use the fitusiran drug product Prefilled Pen if the expiration date has passed.

### A4) Check the Medicine

Look at the medicine through the Window, it should be clear and colorless to pale yellow.

Note: You may see air bubbles, this is normal.

Do not use the fitusiran drug product Prefilled Pen if the liquid is discolored or cloudy, or if it contains visible flakes or particles.

### A5) if Fitusiran Drug Product Prefilled Pen was Stored in the Refrigerator, Warm Up for 30 Minutes.

Lay the fitusiran drug product Prefilled Pen on a clean, flat surface and let it warm up at room temperature between 59° F. to 86° F. (15° C. to 30° C.) for at least 30 minutes.

Do not heat the fitusiran drug product Prefilled Pen or put into direct sunlight.

### B. Choose and Prepare Your Injection Site

B1) Wash Your Hands Well with Soap and Water

### B2) Choose an Injection Site

### Thigh

Stomach except for the 2 inches (5 cm) around your belly button (navel).

A caregiver can also inject in the outer area of your upper arm.

Do not inject into skin that is tender, damaged, has bruises or scars, or into areas with visible veins.

For subcutaneous use only. Do not inject into a vein.

Do not inject through clothes.

### B3) Prepare the Injection Site

Clean the injection site with an alcohol wipe.

Let the skin dry before injecting.

Do not touch the injection site again or blow on it before the injection.

### C. Give the Injection

### C1) Remove Blue Cap

Remove the Blue Cap by pulling it straight off as shown. Do not twist the Blue Cap off.

Do not remove the Blue Cap until you are ready to inject.

Do not touch the Orange Needle Cover with your fingers. The Needle is inside.

Do not put the Blue Cap back on the fitusiran drug product Prefilled Pen after you have removed it.

Note: You may see a droplet on the needle tip. This is normal.

### C2) Place Fitusiran Drug Product Prefilled Pen on Injection Site

Hold the fitusiran drug product Prefilled Pen as shown so you can see the Window.

Place the Orange Needle Cover on the skin at a 90-degree angle.

### C3) Press Down Firmly—Watch Window Turn Fully Yellow

Press down and hold the fitusiran drug product Prefilled Pen firmly against the skin until you cannot see the Orange Needle Cover.

There will be a “click” when the injection starts, and

The Window will start to turn yellow.

Keep pressing the fitusiran drug product Prefilled Pen against the skin and watch the Window.

The Window will turn completely yellow, and

You may hear a 2nd “click.” The injection is complete.

### C4) Remove

After you have completed your injection pull straight up to remove fitusiran drug product Prefilled Pen from the skin. The Orange Needle Cover will cover the needle.

If you see any blood at the site, lightly dab a cotton ball or gauze pad.

Do not rub the skin after the injection.

If the Window does not turn completely yellow, or if it looks like you may not have received a full dose, call your healthcare provider right away.

Do not give a second dose without speaking to your healthcare provider.

### D. Throw Away (Dispose of) Used Fitusiran Drug Product Prefilled Pen

Do not put the Blue Cap back on.

Keep your sharps disposal container out of the reach of children.

### How to Dispose of (Throw Away) Fitusiran Drug Product Prefilled Pen

Put your used fitusiran drug product Prefilled Pen and Blue Cap in an FDA-cleared sharps disposal container right away after use.

Do not dispose of (throw away) fitusiran drug product Prefilled Pen and Blue Cap in your household trash.

If you do not have an FDA-cleared sharps disposal container, you may use a household container that is:
made of a heavy-duty plastic,
can be closed with a tight-fitting, puncture-resistant lid, without sharps being able to come out,
upright and stable during use,
leak-resistant, and
properly labeled to warn of hazardous waste inside the container.

When your sharps disposal container is almost full, you will need to follow your community guidelines for the right way to dispose of your sharps disposal container. There may be state or local laws about how you should throw away used fitusiran drug product Prefilled Pens.

Do not dispose of your used sharps disposal container in your household trash unless your community guidelines permit this.

Do not recycle your used sharps disposal container.

1. A method of routine prophylaxis to prevent or reduce the frequency of bleeding episodes in a human patient having hemophilia A or B with or without factor VIII or factor IX inhibitors, comprising:
(a) subcutaneously administering to the human patient in need thereof fitusiran at a starting dose of 50 mg about once every 2 months;
(b) obtaining a measurement of an antithrombin (AT) level in the human patient;
(c) performing one of the following steps:
(i) if the AT level is 15-35%, repeating step (a),
(ii) if the AT level is >35% after 6 months, subcutaneously administering to the human patient fitusiran at a dose of 50 mg about once every month, or
(iii) if the AT level is <15%, subcutaneously administering to the human patient fitusiran at a dose of 20 mg about once every 2 months;
(d) after administering to the human patient fitusiran at a dose of 20 mg about once every 2 months, obtaining a measurement of an antithrombin (AT) level in the human patient;
(e) performing one of the following steps:
(i) if the AT level is 15-35%, subcutaneously administering to the human patient fitusiran at a dose of 20 mg about once every 2 months,
(ii) if the AT level is >35% after 6 months, subcutaneously administering to the human patient fitusiran at a dose of 20 mg about once every month, or
(iii) if the AT level is <15%, subcutaneously administering to the human patient fitusiran at a dose of 10 mg about once every 2 months;
(f) after administering to the human patient fitusiran at a dose of 10 mg about once every 2 months, obtaining a measurement of an antithrombin (AT) level in the human patient; and
(g) performing one of the following steps:
(i) if the AT level is 15-35%, subcutaneously administering to the human patient fitusiran at a dose of 10 mg about once every 2 months,
(ii) if the AT level is >35% after 6 months, subcutaneously administering to the human patient fitusiran at a dose of 10 mg about once every month, or
(iii) if the AT level is <15%, discontinuing or pausing fitusiran treatment.

2. A method of reducing a risk of hepatotoxicity in a human patient with hemophilia A or B with or without factor VIII or factor IX inhibitors who receives fitusiran for routine prophylaxis to prevent or reduce the frequency of bleeding episodes, comprising:
(a) obtaining a measurement of an antithrombin (AT) level in the human patient, wherein the human patient has previously received fitusiran at a starting dose of 50 mg about once every 2 months;
(b) performing one of the following steps:
(i) if the AT level is 15-35%, subcutaneously administering to the human patient fitusiran at a dose of 50 mg about once every 2 months,
(ii) if the AT level is >35% after 6 months, subcutaneously administering to the human patient fitusiran at a dose of 50 mg about once every month, or
(iii) if the AT level is <15%, subcutaneously administering to the human patient fitusiran at a dose of 20 mg about once every 2 months;
(c) after administering to the human patient fitusiran at a dose of 20 mg about once every 2 months, obtaining a measurement of an antithrombin (AT) level in the human patient;
(d) performing one of the following steps:
(i) if the AT level is 15-35%, subcutaneously administering to the human patient fitusiran at a dose of 20 mg about once every 2 months,
(ii) if the AT level is >35% after 6 months, subcutaneously administering to the human patient fitusiran at a dose of 20 mg about once every month, or
(iii) if the AT level is <15%, subcutaneously administering to the human patient fitusiran at a dose of 10 mg about once every 2 months;
(e) after administering to the human patient fitusiran at a dose of 10 mg about once every 2 months, obtaining a measurement of an antithrombin (AT) level in the human patient; and
(f) performing one of the following steps:
(i) if the AT level is 15-35%, subcutaneously administering to the human patient fitusiran at a dose of 10 mg about once every 2 months,
(ii) if the AT level is >35% after 6 months, subcutaneously administering to the human patient fitusiran at a dose of 10 mg about once every month, or
(iii) if the AT level is <15%, discontinuing or pausing fitusiran treatment.

3. The method of claim 2, wherein reducing the risk of hepatotoxicity comprises reducing the occurrence in the human patient of alanine transaminase (ALT) elevation more than three times the upper limit of normal (ULN), aspartate aminotransferase (AST) elevation more than three times the ULN, severe liver toxicity, liver failure, and/or jaundice.

4. A method of reducing a risk of gallbladder disease in a human patient with hemophilia A or B with or without factor VIII or factor IX inhibitors who receives fitusiran for routine prophylaxis to prevent or reduce the frequency of bleeding episodes, comprising:
(a) obtaining a measurement of an antithrombin (AT) level in the human patient, wherein the human patient has previously received fitusiran at a starting dose of 50 mg about once every 2 months;
(b) performing one of the following steps:
(i) if the AT level is 15-35%, subcutaneously administering to the human patient fitusiran at a dose of 50 mg about once every 2 months,
(ii) if the AT level is >35% after 6 months, subcutaneously administering to the human patient fitusiran at a dose of 50 mg about once every month, or
(iii) if the AT level is <15%, subcutaneously administering to the human patient fitusiran at a dose of 20 mg about once every 2 months;
(c) after administering to the human patient fitusiran at a dose of 20 mg about once every 2 months, obtaining a measurement of an antithrombin (AT) level in the human patient;
(d) performing one of the following steps:
(i) if the AT level is 15-35%, subcutaneously administering to the human patient fitusiran at a dose of 20 mg about once every 2 months,
(ii) if the AT level is >35% after 6 months, subcutaneously administering to the human patient fitusiran at a dose of 20 mg about once every month, or
(iii) if the AT level is <15%, subcutaneously administering to the human patient fitusiran at a dose of 10 mg about once every 2 months;
(e) after administering to the human patient fitusiran at a dose of 10 mg about once every 2 months, obtaining a measurement of an antithrombin (AT) level in the human patient; and
(f) performing one of the following steps:
(i) if the AT level is 15-35%, subcutaneously administering to the human patient fitusiran at a dose of 10 mg about once every 2 months,
(ii) if the AT level is >35% after 6 months, subcutaneously administering to the human patient fitusiran at a dose of 10 mg about once every month, or
(iii) if the AT level is <15%, discontinuing or pausing fitusiran treatment.

5. The method of claim 4, wherein reducing the risk of gallbladder disease comprises reducing the occurrence in the human patient of cholecystitis, cholelithiasis, or a need for cholecystectomy.

6. A method of reducing thrombotic risk in a human patient with hemophilia A or B with or without factor VIII or factor IX inhibitors who receives fitusiran for routine prophylaxis to prevent or reduce the frequency of bleeding episodes, comprising:
(a) obtaining a measurement of an antithrombin (AT) level in the human patient, wherein the human patient has previously received fitusiran at a starting dose of 50 mg about once every 2 months;
(b) performing one of the following steps:
(i) if the AT level is 15-35%, subcutaneously administering to the human patient fitusiran at a dose of 50 mg about once every 2 months,
(ii) if the AT level is >35% after 6 months, subcutaneously administering to the human patient fitusiran at a dose of 50 mg about once every month, or
(iii) if the AT level is <15%, subcutaneously administering to the human patient fitusiran at a dose of 20 mg about once every 2 months;
(c) after administering to the human patient fitusiran at a dose of 20 mg about once every 2 months, obtaining a measurement of an antithrombin (AT) level in the human patient;
(d) performing one of the following steps:
(i) if the AT level is 15-35%, subcutaneously administering to the human patient fitusiran at a dose of 20 mg about once every 2 months,
(ii) if the AT level is >35% after 6 months, subcutaneously administering to the human patient fitusiran at a dose of 20 mg about once every month, or
(iii) if the AT level is <15%, subcutaneously administering to the human patient fitusiran at a dose of 10 mg about once every 2 months;
(e) after administering to the human patient fitusiran at a dose of 10 mg about once every 2 months, obtaining a measurement of an antithrombin (AT) level in the human patient; and
(f) performing one of the following steps:
(i) if the AT level is 15-35%, subcutaneously administering to the human patient fitusiran at a dose of 10 mg about once every 2 months,
(ii) if the AT level is >35% after 6 months, subcutaneously administering to the human patient fitusiran at a dose of 10 mg about once every month, or
(iii) if the AT level is <15%, discontinuing or pausing fitusiran treatment.

7. The method of claim 6, wherein reducing thrombotic risk comprises reducing the occurrence in the human patient of cerebrovascular accident, cerebral artery embolism, spinal vascular disorder, atrial thrombosis, cerebral venous sinus thrombosis, thrombosis, thrombosis on papillae of an eye, deep venous thrombosis of an arm, subclavian vein thrombosis, superficial venous thrombosis of an arm, cerebral infarction, embolic stroke, or postoperative venous thrombosis.

8. A method of monitoring an antithrombin level in a human patient with hemophilia A or B with or without factor VIII or factor IX inhibitors who receives fitusiran for routine prophylaxis to prevent or reduce the frequency of bleeding episodes, comprising:
(a) obtaining a measurement of an antithrombin (AT) level in the human patient, wherein the human patient has previously received fitusiran at a starting dose of 50 mg about once every 2 months;
(b) performing one of the following steps:
(i) if the AT level is 15-35%, subcutaneously administering to the human patient fitusiran at a dose of 50 mg about once every 2 months,
(ii) if the AT level is >35% after 6 months, subcutaneously administering to the human patient fitusiran at a dose of 50 mg about once every month, or
(iii) if the AT level is <15%, subcutaneously administering to the human patient fitusiran at a dose of 20 mg about once every 2 months;
(c) after administering to the human patient fitusiran at a dose of 20 mg about once every 2 months, obtaining a measurement of an antithrombin (AT) level in the human patient;
(d) performing one of the following steps:
(i) if the AT level is 15-35%, subcutaneously administering to the human patient fitusiran at a dose of 20 mg about once every 2 months,
(ii) if the AT level is >35% after 6 months, subcutaneously administering to the human patient fitusiran at a dose of 20 mg about once every month, or
(iii) if the AT level is <15%, subcutaneously administering to the human patient fitusiran at a dose of 10 mg about once every 2 months;
(e) after administering to the human patient fitusiran at a dose of 10 mg about once every 2 months, obtaining a measurement of an antithrombin (AT) level in the human patient; and
(f) performing one of the following steps:
(i) if the AT level is 15-35%, subcutaneously administering to the human patient fitusiran at a dose of 10 mg about once every 2 months,
(ii) if the AT level is >35% after 6 months, subcutaneously administering to the human patient fitusiran at a dose of 10 mg about once every month, or
(iii) if the AT level is <15%, discontinuing or pausing fitusiran treatment.

9. The method of claim 8, wherein the antithrombin level is monitored using a kinetic or chromogenic assay, wherein the chromogenic assay quantifies functionally active AT in human citrated plasma based on the inhibition of an excess of factor Xa by AT.

10. A method of reducing annualized bleeding rate in a human patient with hemophilia A or B with or without factor VIII or factor IX inhibitors who receives fitusiran for routine prophylaxis to prevent or reduce the frequency of bleeding episodes, comprising:
(a) obtaining a measurement of an antithrombin (AT) level in the human patient, wherein the human patient has previously received fitusiran at a starting dose of 50 mg about once every 2 months;
(b) performing one of the following steps:
(i) if the AT level is 15-35%, subcutaneously administering to the human patient fitusiran at a dose of 50 mg about once every 2 months,
(ii) if the AT level is >35% after 6 months, subcutaneously administering to the human patient fitusiran at a dose of 50 mg about once every month, or
(iii) if the AT level is <15%, subcutaneously administering to the human patient fitusiran at a dose of 20 mg about once every 2 months;
(c) after administering to the human patient fitusiran at a dose of 20 mg about once every 2 months, obtaining a measurement of an antithrombin (AT) level in the human patient;
(d) performing one of the following steps:
(i) if the AT level is 15-35%, subcutaneously administering to the human patient fitusiran at a dose of 20 mg about once every 2 months,
(ii) if the AT level is >35% after 6 months, subcutaneously administering to the human patient fitusiran at a dose of 20 mg about once every month, or
(iii) if the AT level is <15%, subcutaneously administering to the human patient fitusiran at a dose of 10 mg about once every 2 months;
(e) after administering to the human patient fitusiran at a dose of 10 mg about once every 2 months, obtaining a measurement of an antithrombin (AT) level in the human patient; and
(f) performing one of the following steps:
(i) if the AT level is 15-35%, subcutaneously administering to the human patient fitusiran at a dose of 10 mg about once every 2 months,
(ii) if the AT level is >35% after 6 months, subcutaneously administering to the human patient fitusiran at a dose of 10 mg about once every month, or
(iii) if the AT level is <15%, discontinuing or pausing fitusiran treatment.

11. The method of any one of claims 1-10, further comprising administering an effective amount of a replacement factor or bypassing agent (BPA) to treat a bleeding episode that occurs eight or more days after the first fitusiran dose, wherein the effective amount of the replacement factor or BPA is reduced as compared to the recommended effective amount of the replacement factor or BPA for human patients who are not on fitusiran therapy.

12. The method of claim 11, wherein the effective amount of the replacement factor or BPA is reduced to one half of a weight-based dose of the recommended effective amount of the replacement factor or BPA for human patients who are not on fitusiran therapy, administered at double the dosing frequency recommended for human patients who are not on fitusiran therapy.

13. The method of any one of claims 1-12, wherein:
(a) the human patient is a hemophilia A patient without factor VIII inhibitors, and wherein the replacement factor is factor VIII (FVIII) and a single dose of FVIII is no more than 20 IU/kg; or
(b) the human patient is a hemophilia B patient without factor IX inhibitors, and wherein the replacement factor is factor IX (FIX) and a single dose of FIX is no more than 30 IU/kg.

14. The method of any one of claims 1-13, wherein the human patient is a hemophilia A patient with factor VIII inhibitors or a hemophilia B patient with factor IX inhibitors, wherein the BPA is activated prothrombin complex concentrate (aPCC) and a single dose of aPCC is no more than 50 U/kg, or wherein the BPA is recombinant factor VIIa (rFVIIa) and a single dose of rFVIIa is no more than 45 g/kg.

15. The method of any one of claims 1-10, further comprising administering an effective amount of a replacement factor or bypassing agent (BPA) to treat a bleeding episode that occurs seven or fewer days after the first fitusiran dose, wherein the effective amount of the replacement factor or BPA is administered according to the human patient's prior dosing regimen of replacement factor or BPA.

16. The method of any one of claims 1-15, wherein the human patient has been on prophylactic treatment with a replacement factor or a bypassing agent (BPA), and wherein the method comprises terminating the prophylactic replacement factor or BPA treatment in the human patient within about two weeks or about 14 days or about one week or about seven days of the first dose of fitusiran.

17. The method of any one of claims 1-16, comprising obtaining a measurement of AT level in the human patient about every four weeks, about every eight weeks, about every month, about every two months, about every four months, about every six months, or about every 12 months, and/or at four weeks or one month, twelve weeks or three months, 20 weeks or five months, and 24 weeks or six months following administration of the starting dose or following administration of a modified dose as compared to the prior dose.

18. The method of any one of claims 1-17, wherein the obtaining a measurement of the AT level in the human patient comprises use of a kinetic or chromogenic assay.

19. The method of any one of claims 1-18, further comprising subcutaneously administering fitusiran to the human patient at a dose amount and a dosing frequency sufficient to maintain the AT level in the human patient at 15-35%.

20. The method of claim 19, further comprising subcutaneously administering fitusiran at:
10 mg QM or Q4W,
10 mg Q2M or Q8W,
20 mg QM or Q4W,
20 mg Q2M or Q8W,
50 mg QM or Q4W, or
50 mg Q2M or Q8W.

21. The method of any one of claims 1-20, wherein the human patient does not have
(i) clinically significant liver disease,
(ii) ALT >1.5× upper limit of normal reference range (ULN),
(iii) AST >1.5× upper limit of normal reference range (ULN),
(iv) hepatitis C,
(v) hepatitis A,
(vi) hepatitis E,
(vii) hepatitis B,
(viii) acute and recurrent gallbladder disease,
(ix) symptomatic gallbladder disease,
(x) established thrombophilic conditions,
(xi) a prior history of thrombosis,
(xii) an indwelling venous catheter,
(xiii) persistent AT activity <15%, or
(xiv) known hepatic impairment.

22. A pharmaceutical composition, comprising fitusiran at a concentration of about 35 mg/ml to about 45 mg/mL and phosphate buffered saline (PBS) at a concentration of about 1 mM to about 10 mM,
wherein the pH and the osmolality of the pharmaceutical composition are suitable for subcutaneous administration to a subject, and
wherein the composition comprises about 7.67 to about 7.69 mg/mL sodium chloride, about 0.932 to about 0.934 mg/mL dibasic sodium phosphate (heptahydrate), and about 0.208 to about 0.211 mg/mL monobasic sodium phosphate (monohydrate), wherein the pH of the composition is between about 5.0 to about 8.0.

23. A pharmaceutical composition comprising fitusiran, wherein:
(a) 0.5 mL of the pharmaceutical composition comprises about 50 mg of fitusiran (equivalent to about 53.0 mg fitusiran sodium), about 0.585 mg dibasic sodium phosphate, about 0.044 mg monobasic sodium phosphate, about 2.455 mg sodium chloride, and water for injection, wherein the pH of the pharmaceutical composition is about 7.0;
(b) 0.5 mL of the pharmaceutical composition comprises about 20 mg of fitusiran (equivalent to about 21.2 mg fitusiran sodium), about 0.467 mg dibasic sodium phosphate, about 0.105 mg monobasic sodium phosphate, about 3.836 mg sodium chloride, and water for injection, wherein the pH of the pharmaceutical composition is about 7.0; or
(c) 0.2 mL of the pharmaceutical composition comprises about 20 mg of fitusiran (equivalent to about 21.2 mg fitusiran sodium), about 0.234 mg dibasic sodium phosphate, about 0.018 mg monobasic sodium phosphate, about 0.982 mg sodium chloride, and water for injection.

24. A method of treating hemophilia A or hemophilia B prophylactically in a patient in need thereof, comprising administering to the patient the pharmaceutical composition of claim 22 or 23, wherein administering the pharmaceutical composition comprises subcutaneous injection of the pharmaceutical composition from a prefilled syringe or a prefilled vial containing the pharmaceutical composition.


SEQUENCE LISTING (4 sequences)

SEQ ID NO: 1 - RNA, 21 nt, synthetic construct
  source 1..21: mol_type = other RNA
  modified_base 1: mod_base = OTHER; note = 2'-fluoroguanosine (i.e., 2'-deoxy-2'-fluoroguanosine)
  modified_base 2: mod_base = gm
  modified_base 3: mod_base = OTHER; note = 2'-fluorouridine (i.e., 2'-deoxy-2'-fluorouridine)
  modified_base 13: mod_base = OTHER; note = 2'-fluorouridine (i.e., 2'-deoxy-2'-fluorouridine)
  modified_base 17: mod_base = OTHER; note = 2'-fluorouridine (i.e., 2'-deoxy-2'-fluorouridine)
  modified_base 4: mod_base = um
  modified_base 12: mod_base = um
  modified_base 14: mod_base = um
  modified_base 18: mod_base = um
  modified_base 19: mod_base = OTHER; note = 2'-fluorocytidine (i.e., 2'-deoxy-2'-fluorocytidine)
  modified_base 5: mod_base = OTHER; note = 2'-fluoroadenosine (i.e., 2'-deoxy-2'-fluoroadenosine)
  modified_base 11: mod_base = OTHER; note = 2'-fluoroadenosine (i.e., 2'-deoxy-2'-fluoroadenosine)
  modified_base 15: mod_base = OTHER; note = 2'-fluoroadenosine (i.e., 2'-deoxy-2'-fluoroadenosine)
  modified_base 21: mod_base = OTHER; note = 2'-fluoroadenosine (i.e., 2'-deoxy-2'-fluoroadenosine)
  modified_base 6: mod_base = OTHER; note = 2'-O-methyladenosine
  modified_base 7: mod_base = OTHER; note = 2'-fluorocytidine (i.e., 2'-deoxy-2'-fluorocytidine)
  modified_base 8: mod_base = OTHER; note = 2'-O-methyladenosine
  modified_base 9..10: mod_base = OTHER; note = 2'-fluorocytidine (i.e., 2'-deoxy-2'-fluorocytidine)
  modified_base 16: mod_base = cm
  modified_base 1..3: mod_base = OTHER; note = 3'-5' phosphorothioate linkage sodium salt
  modified_base 3..21: mod_base = OTHER; note = 3'-5' phosphodiester linkage sodium salt
  modified_base 20: mod_base = OTHER; note = 2'-O-methyladenosine
ggttaacaccatttacttcaa

SEQ ID NO: 2 - RNA, 23 nt, synthetic construct
  source 1..23: mol_type = other RNA
  modified_base 1: mod_base = um
  modified_base 2: mod_base = OTHER; note = 2'-fluorouridine (i.e., 2'-deoxy-2'-fluorouridine)
  modified_base 3: mod_base = gm
  modified_base 4: mod_base = OTHER; note = 2'-fluoroadenosine (i.e., 2'-deoxy-2'-fluoroadenosine)
  modified_base 5: mod_base = OTHER; note = 2'-O-methyladenosine
  modified_base 6: mod_base = OTHER; note = 2'-fluoroguanosine (i.e., 2'-deoxy-2'-fluoroguanosine)
  modified_base 7: mod_base = um
  modified_base 8: mod_base = OTHER; note = 2'-fluoroadenosine (i.e., 2'-deoxy-2'-fluoroadenosine)
  modified_base 9: mod_base = OTHER; note = 2'-O-methyladenosine
  modified_base 10: mod_base = OTHER; note = 2'-fluoroadenosine (i.e., 2'-deoxy-2'-fluoroadenosine)
  modified_base 11: mod_base = um
  modified_base 12: mod_base = gm
  modified_base 13: mod_base = gm
  modified_base 14: mod_base = OTHER; note = 2'-fluorouridine (i.e., 2'-deoxy-2'-fluorouridine)
  modified_base 15: mod_base = gm
  modified_base 16: mod_base = OTHER; note = 2'-fluorouridine (i.e., 2'-deoxy-2'-fluorouridine)
  modified_base 17: mod_base = um
  modified_base 18: mod_base = OTHER; note = 2'-fluoroadenosine (i.e., 2'-deoxy-2'-fluoroadenosine)
  modified_base 19: mod_base = OTHER; note = 2'-O-methyladenosine
  modified_base 20: mod_base = OTHER; note = 2'-fluorocytidine (i.e., 2'-deoxy-2'-fluorocytidine)
  modified_base 21: mod_base = cm
  modified_base 22: mod_base = OTHER; note = 2'-O-methyladenosine
  modified_base 23: mod_base = gm
  modified_base 1..3: mod_base = OTHER; note = 3'-5' phosphorothioate linkage sodium salt
  modified_base 21..23: mod_base = OTHER; note = 3'-5' phosphorothioate linkage sodium salt
  modified_base 3..21: mod_base = OTHER; note = 3'-5' phosphodiester linkage sodium salt
ttgaagtaaatggtgttaaccag

SEQ ID NO: 3 - RNA, 21 nt, synthetic construct
  source 1..21: mol_type = other RNA
  modified_base 1: mod_base = OTHER; note = 2'-fluoroguanosine (i.e., 2'-deoxy-2'-fluoroguanosine)
  modified_base 3: mod_base = OTHER; note = 2'-fluorouridine (i.e., 2'-deoxy-2'-fluorouridine)
  modified_base 5: mod_base = OTHER; note = 2'-fluoroadenosine (i.e., 2'-deoxy-2'-fluoroadenosine)
  modified_base 7: mod_base = OTHER; note = 2'-fluorocytidine (i.e., 2'-deoxy-2'-fluorocytidine)
  modified_base 9..10: mod_base = OTHER; note = 2'-fluorocytidine (i.e., 2'-deoxy-2'-fluorocytidine)
  modified_base 11: mod_base = OTHER; note = 2'-fluoroadenosine (i.e., 2'-deoxy-2'-fluoroadenosine)
  modified_base 13: mod_base = OTHER; note = 2'-fluorouridine (i.e., 2'-deoxy-2'-fluorouridine)
  modified_base 15: mod_base = OTHER; note = 2'-fluoroadenosine (i.e., 2'-deoxy-2'-fluoroadenosine)
  modified_base 17: mod_base = OTHER; note = 2'-fluorouridine (i.e., 2'-deoxy-2'-fluorouridine)
  modified_base 19: mod_base = OTHER; note = 2'-fluorocytidine (i.e., 2'-deoxy-2'-fluorocytidine)
  modified_base 21: mod_base = OTHER; note = 2'-fluoroadenosine (i.e., 2'-deoxy-2'-fluoroadenosine)
  modified_base 1..3: mod_base = OTHER; note = phosphorothioate linkage
ggttaacaccatttacttcaa

SEQ ID NO: 4 - RNA, 23 nt, synthetic construct
  source 1..23: mol_type = other RNA
  modified_base 2: mod_base = OTHER; note = 2'-fluorouridine (i.e., 2'-deoxy-2'-fluorouridine)
  modified_base 4: mod_base = OTHER; note = 2'-fluoroadenosine (i.e., 2'-deoxy-2'-fluoroadenosine)
  modified_base 6: mod_base = OTHER; note = 2'-fluoroguanosine (i.e., 2'-deoxy-2'-fluoroguanosine)
  modified_base 8: mod_base = OTHER; note = 2'-fluoroadenosine (i.e., 2'-deoxy-2'-fluoroadenosine)
  modified_base 10: mod_base = OTHER; note = 2'-fluoroadenosine (i.e., 2'-deoxy-2'-fluoroadenosine)
  modified_base 14: mod_base = OTHER; note = 2'-fluorouridine (i.e., 2'-deoxy-2'-fluorouridine)
  modified_base 16: mod_base = OTHER; note = 2'-fluorouridine (i.e., 2'-deoxy-2'-fluorouridine)
  modified_base 18: mod_base = OTHER; note = 2'-fluoroadenosine (i.e., 2'-deoxy-2'-fluoroadenosine)
  modified_base 20: mod_base = OTHER; note = 2'-fluorocytidine (i.e., 2'-deoxy-2'-fluorocytidine)
  modified_base 1..3: mod_base = OTHER; note = phosphorothioate linkage
  modified_base 21..23: mod_base = OTHER; note = phosphorothioate linkage
ttgaagtaaatggtgttaaccag